(19) United States
(12) Patent Application Publication
(10) Pub. No.: US 2004/0009534 A1
(43) Pub. Date: Jan. 15, 2004

(54) Protein analysis

(75) Inventors: Aaron K. Sato, Somerville, MA (US); Bruce M. Dawson, Wilmington, NC (US)

Correspondence Address: FISH & RICHARDSON PC, 225 FRANKLIN ST, BOSTON, MA, 02110, US

(73) Assignee: DYAX CORPORATION

(21) Appl. No.: 10/462,262

(22) Filed: Jun. 16, 2003

Related U.S. Application Data
(60) Provisional application No. 60/388,642, filed on Jun. 14, 2002.

(51) Int. Cl. G01N 33/53

(52) U.S. Cl. 435/7.1

(57) ABSTRACT

Disclosed, inter alia, is a method of evaluating a sample that includes a serum protein and one or more one or more compounds physically associated with the serum protein. The method can include using a peptide ligand that specifically interacts with the serum protein to analyze a complex formed by the serum protein and its associated compounds.

### RELATED APPLICATIONS

This application claims benefit of priority to U.S. Provisional Patent Application Serial No. 60/388,642, filed Jun. 14, 2002, the contents of which are incorporated herein by reference.

### BACKGROUND

This application relates to the analysis of proteins, including serum proteins.

Serum is the blood-derived fluid that remains after blood has clotted. The more abundant serum proteins include serum albumin and antibodies (e.g., IgG, IgM, and the like). Other proteins that can be present in serum include: transferrin, α-macroglobulins, ferritin, apolipoproteins, transthyretin, protease inhibitors, retinol binding protein, thiostatin, α-fetoprotein, vitamin-D binding protein, and afamin (see, e.g., U.S. Pat. No. 5,767,243).

The most abundant protein component in circulating blood of mammalian species is serum albumin, which is normally present at a concentration of approximately 3 to 4.5 grams per 100 ml of whole blood. Serum albumin is a blood protein of approximately 70 kilo-Daltons which provides several important functions in the circulatory system. For instance, it functions as a transporter of a variety of organic molecules found in the blood, as the main transporter of various metabolites such as fatty acids, hematin, and bilirubin, and, owing to its abundance, as an osmotic regulator of the circulating blood. It also has a broad affinity for small, negatively charged aromatic compounds. These binding functions enable serum albumin to serve as the principal carrier of fatty acids that are otherwise insoluble in circulating plasma. Likewise, it can sequester oxygen free radicals and to inactivate toxic lipophilic metabolites such as bilirubin. It can also form covalent adducts with pyridoxal phosphate cysteine, glutathione, and various metals, such as Cu(II), Ni(II) Hg(II), Ag(II), and Au(I).

Serum albumin can also bind to drugs that are present in the body. Indeed, one indicator of the efficacy of a drug is its affinity for serum albumin or other serum proteins. Binding to serum albumin can affect the overall distribution, metabolism, and bioavailability of many drugs. At least in some cases, unusually high affinity to serum albumin has been associated with the failure of candidate drugs.

It is also known to conjugate drugs to serum albumin to extend their half-life and distribution. Recently, chimeric albumin molecules such as HSA-CD4 and HSA-Cu,Zn-superoxide dismutase have been utilized to increase the half-life and distribution and to reduce the immunogenicity of these potential protein therapeutics.

### SUMMARY

In one aspect, the invention features a method that includes: providing a sample that includes (i) a serum albumin, (ii) one or more compounds physically associated with the serum albumin and (iii) a serum albumin-binding agent; allowing the serum albumin-binding agent to bind to the serum albumin to form a complex; separating the complex from one or more components of the sample; and evaluating one or more of the physically associated compounds. The method can be used to evaluating a sample. The method can further include separating one or more of the physically associated compounds from the serum albumin, e.g., prior to the evaluating. In one embodiment, the serum albumin is a human serum albumin.

In an embodiment, the serum albumin-binding agent has one or more of the following properties: is synthetic; includes a protein other than an antibody or antibody derivative; is non-naturally occurring; is free of an immunoglobulin variable domain; includes a peptide that independently binds to serum albumin.

In the latter case, the peptide can include at least one intra-molecular disulfide bonds, e.g., one or two intra-molecular disulfide bonds. The peptide can include a peptide described herein, e.g., DX-321, DX-321-A, DX-321-B, DX-236, DX-236-A, or DX-236B, or a variant thereof, or a peptide described in U.S. Published application 2003/0069395; Ser. No. 10/094,401; Ser. No. 60/331,352; or Ser. No. 60/292,975, or a variant thereof. Particular variants include: functional variants having between one and six substitutions (e.g., between one and four, e.g., one, two, three, or four), e.g., conservative substitutions, truncations, chemically modified forms, peptido-mimetics, and substitutions with non-naturally occurring residues. The peptide may also include be a functional variant with between one and four insertions or deletions, e.g., one , two, three or four. The peptide can be a peptide ligand that competes for binding to serum albumin with a ligand described herein, or a ligand binding an epitope that overlaps with an epitope bound by a ligand described herein. The peptide ligand can be a ligand isolated by screening a display library. The peptide that independently binds to serum albumin can be less than 32, 28, 24, 20, or 16 amino acids in length, or between 12 and 32, 8 and 16, or 12 and 24 amino acids in length.

In one embodiment, the serum albumin-binding agent is coupled to an insoluble support, e.g., a bead (such as a magnetic bead), a matrix (such as a chromatography matrix, agarose, or a porous material), or a planar surface. For example, the support may include a planar surface, and the serum albumin-binding agent is immobilized to a discrete address on the planar surface. The planar surface can also include a second binding agent at a second discrete address, e.g., another serum albumin-binding agent or an agent that binds to a different serum protein.

The serum albumin-binding agent can have a binding affinity (K_{D}) of less than 5, 4, 2, 1, 0.5, 0.1 μM, or less than 50, 10, 5, or 0.5 nM and/or of greater than 0.05, 0.5, 5, or 50 nM, 0.001, 0.1, or 0.1 μM, and ranges therebetween. In one embodiment, the serum albumin-binding agent binds to serum albumin under physiological conditions. In one embodiment, the serum albumin-binding agent and the serum albumin preferentially dissociate at least in solutions above pH 8, 8.5, or 9, e.g., between pH 8 and 11; pH 8 and 10.5; pH 8 and 10; pH 8.5 and 10; or pH 8.7 and 9.5. In another embodiment, the serum albumin-binding agent and the serum albumin preferentially dissociate at least in solutions below pH 6, 5.5, or 6, e.g., between pH 4 and 6; pH 4.6 and 6.5; pH 5 and 6.5; or pH 4.7 and 6.0.

In one embodiment, the serum albumin-binding agent is less than 7, 5, 3, or 2 kDa molecular weight or between 1.5 and 7 or 2 and 6 kDa molecular weight.

The serum albumin-binding agent may bind to serum albumin from a plurality of species, e.g., a plurality of mammalian species, e.g., human and mouse. In another embodiment, the serum albumin-binding agent binds to human serum albumin but not murine serum albumin nor bovine serum albumin.

In one embodiment, at least one of the evaluated physically associated compounds is non-covalently associated with the serum albumin. Such compounds may be directly or indirectly physically associated with the serum albumin. An indirect interaction may be bridged by one or more compounds, at least one of which is directly associated with the serum albumin. In another embodiment, at least one of the evaluated physically associated compounds is covalently associated with the serum albumin. In some embodiments, at least one of the evaluated physically associated compounds is covalently associated and at least another is non-covalently associated.

The method can include further including separating the at least one non-covalently associated compounds from the serum albumin, e.g., prior to the evaluating. The separating from the serum albumin can include covalently attaching the serum albumin to an insoluble support, e.g., a matrix, a particle, or a surface. For example, the covalent attachment can be to a free cysteine of the serum albumin. The covalent attachment can be formed using a thiol reactive group, e.g., a halogen derivative (such as iodoacetamide), a maleimide, or a thiol exchange reagent (e.g., a pyridyl disulfide).

The separating can include denaturing the serum albumin, e.g., using a chaotrope, an organic solvent, high or low pH, or heat. In another embodiment, where at least one of the evaluated covalently associated compounds is protease resistant (e.g., includes a non-proteinaceous component), the separating can include degrading the serum albumin, e.g., using a protease.

The evaluating can include one or more of: gel electrophoresis, mass spectroscopy, chromatography, protein sequencing, detecting a label (e.g., a radioactive, fluorescent, enzymatic, or chemical label), detecting a given compound using an affinity reagent specific for the given compound, or another method described herein. The affinity reagent may be an antibody. For example, the detecting can include performing an immuno-blot or an immuno-precipitation. Information from the evaluating can be recorded on a machine-readable medium, transmitted across a computer network, or stored in a database.

The subject of the evaluating can include a proteinaceous or a non-proteinaceous chemical compound. For example, the subject can include a peptide, a polypeptide, a protein complex, or a drug. In one embodiment, the compound is other than one or more of the compounds in Table 1 or Table 2, e.g., a compound other than a fatty acid, hematin, bilirubin, or an exogenous compound.

In one embodiment, the evaluating includes eluting an associated compound from the serum albumin by competition using a synthetic affinity ligand specific for an epitope of the serum albumin or a natural compound (e.g., a fatty acid, hematin, and bilirubin) that binds to the serum albumin. The natural compound can include a negatively charged aromatic group having a molecular weight of less than 500 Daltons.

In one embodiment, the serum albumin is an artificial mutant of a naturally-occurring serum albumin. For example, the serum albumin can be fused to a heterologous polypeptide or covalently coupled to a therapeutic agent (e.g., a cytotoxic drug).

The method can further include digitally recording information that (i) indicates the presences or absence of a given compound among the evaluated one or more physically associated compounds, or (ii) describes the one or more physically associated compounds.

In one embodiment, the sample is obtained from a subject, e.g., a human, e.g., a patient. The sample may include blood or serum. In another example, the sample is obtained from a biopsy, e.g., obtained from a tumor, a region adjacent to a tumor, or a lymph node. The subject may be treated with a therapeutic composition prior to obtaining the sample.

In one embodiment, one or more of the evaluated physically associated compounds is an endogenous compound. In another embodiment, one or more of the evaluated physically associated compounds is a component of the therapeutic composition.

In one embodiment, the method further includes providing a second sample, and evaluating one or more of the physically associated compounds in the second sample. The method can further include comparing results of evaluating the one or more of the physically associated compounds for the first sample to the second sample.

In one embodiment, the first and second samples are obtained from a first and a second subject, respectively. In one example, the first subject and second subject are respectively treated with an agent and untreated with the agent, e.g., a small molecule. The agent may be administered parenterally. In another example, the first subject and second subject are subjected to different environmental conditions. In still another example, the first subject is a reference subject and the second subject is an experimental subject. In another example, the first subject is a reference subject and the second subject is an affected and/or diseased subject. In still another example, the first and second samples are obtained from the same subject, e.g., at different times, e.g., at different times during a treatment.

The results can be recorded in a machine or on machine-readable media. For example, the results are stored in a computer database.

In one embodiment, the results for the first and second samples are compared to a reference sample. In one embodiment, results for the first and second samples are compared to a database that includes records for samples, each sample record being associated with information about the sample (e.g., origin, disease, environmental condition, physiological condition, and so forth).

In another aspect, the invention features a method that includes providing a sample that includes a serum albumin, one or more compounds associated with the serum albumin, and a component that does not associate with the serum albumin; contacting the sample to an affinity ligand specific for the serum albumin; and separating the un-associated component from a composition that includes the serum albumin and one or more of the associated compounds, thereby providing a serum albumin-associated compound. The method can be used to provide a serum albumin-associated compound. The method can include other features described herein. The invention also provides a composition prepared by the above method or a method described herein.

The method can further include separating the associated compound from the serum albumin to provide a serum-albumin free preparation. The invention also features a serum-albumin free preparation prepared according to the above method or another method described herein.

In another aspect, the invention features a method that includes providing (e.g., receiving or obtaining) a first and second sample that each includes a serum protein (e.g., a serum albumin, a soluble immunoglobulin, or other serum protein); evaluating each sample for associated compound(s), if present, e.g., according to a method described herein; and comparing results of the evaluating for the first and second samples. The method can further include, prior to the evaluating, isolating the serum protein and compounds associated with the serum protein from each sample. The separating can include covalently attaching the serum protein to an insoluble matrix. The serum protein can be an abundant serum protein, e.g., a serum protein that is forms at least 0.01, 0.05, or 0.1% of the blood serum.

In one embodiment, the first and second samples are obtained from a first and a second subject, respectively. In one example, the first subject and second subject are respectively treated with an agent and untreated with the agent, e.g., a small molecule. The agent may be administered parenterally. In another example, the first subject and second subject are subjected to different environmental conditions. In still another example, the first subject is a reference subject and the second subject is an experimental subject. In another example, the first subject is a reference subject and the second subject is an affected and/or diseased subject.

The results can be recorded in a machine or on machine-readable media. For example, the results are stored in a computer database.

In one embodiment, the results for the first and second samples are compared to a reference sample. In one embodiment, results for the first and second samples are compared to a database that includes records for samples, each sample record being associated with information about the sample (e.g., origin, disease, environmental condition, physiological condition, and so forth).

The method can also include other features described herein.

In another aspect, the invention features a method that includes: providing a sample that includes (i) a soluble immunoglobulin protein that includes at least one immunoglobulin domain (ii) one or more compounds physically associated with the soluble immunoglobulin protein and (iii) a peptide immunoglobulin-binding agent; allowing the immunoglobulin-binding agent to bind to the soluble immunoglobulin protein to form a complex that includes one or more compounds physically associated with the soluble immunoglobulin protein; separating the complex from one or more components of the sample; and evaluating one or more of the physically associated compounds. The method can be used to evaluate a sample.

In one embodiment, the soluble immunoglobulin protein is a naturally-occurring protein, e.g., IgG, IgM, IgA, IgE, or IgD. In another embodiment, the soluble immunoglobulin protein is a Fab or single-chain antibody. Such protein may include at least one synthetic complementarity determining region (CDR).

In one embodiment, the one or more physically associated compounds includes an antigen of a pathogen.

The sample can be obtained from a subject having an infection, immunological disorder (e.g., an auto-immune disorder), or a genetic disorder. The subject may also be a normal subject.

In an embodiment, the immunoglobulin-binding agent has one or more of the following properties: is synthetic; includes a protein other than an antibody or antibody derivative; is non-naturally occurring; is free of an immunoglobulin variable domain; includes a peptide that independently binds to immunoglobulin. The immunoglobulin-binding agent can bind to the Fc region, to a constant domain (e.g., CH1, CH2, CH3, CH4, or CL), or to a framework region of a variable domain. In a principal embodiment, the immunoglobulin binding agent does not bind to the antigen-binding site of an immunoglobulin.

The peptide can include one or more intra-molecular disulfide bonds, e.g., one or two intra-molecular disulfide bonds. In the case of an immunoglobulin binding agent that binds to an Fc region, the peptide can include a peptide described herein, e.g., DX249, DX249-A, DX249-B, DX253, DX253-A, DX253-B, DX398, DX398-A, DX398-B or a variant thereof, or a compound described in Ser. No. 10/125,869, filed Apr. 18, 2002, or a variant thereof. Exemplary variants include: functional variants having between one and six substitutions, e.g., conservative substitutions, truncations, chemically modified forms, peptido-mimetics, and substitutions with non-naturally occurring residues. The peptide can be a peptide ligand that competes for binding to an immunoglobulin with a ligand described herein, or a ligand binding an epitope that overlaps with an epitope bound by a ligand described herein. The peptide ligand can be a ligand isolated by screening a display library. The peptide that independently binds to an immunoglobulin can be less than 32, 28, 24, 20, or 16 amino acids in length, or between 12 and 32, 8 and 16, or 12 and 24 amino acids in length.

In one embodiment, the immunoglobulin-binding agent is coupled to an insoluble support, e.g., a bead (such as a magnetic bead), a matrix (such as a chromatography matrix), or a planar surface. For example, the support may include a planar surface, and the immunoglobulin-binding agent is immobilized to a discrete address on the planar surface. The planar surface can also include a second binding agent at a second discrete address, e.g., another immunoglobulin-binding agent or an agent that binds to a different serum protein.

The immunoglobulin-binding agent can have a binding affinity (K_{D}) of less than 5, 4, 2, 1, 0.5, or 0.1 μM and/or of greater than 0.001, 0.1, or 0.1 μM, and ranges therebetween. In one embodiment, the immunoglobulin-binding agent binds to immunoglobulin under physiological conditions.

In one embodiment, the immunoglobulin-binding agent is less than 7, 5, 3, or 2 kDa molecular weight or between 1.5 and 7 or 2 and 6 kDa molecular weight.

The immunoglobulin-binding agent may bind to immunoglobulins from a plurality of species, e.g., a plurality of mammalian species, e.g., human and mouse. In another embodiment, the immunoglobulin-binding agent binds to a human immunoglobulin but not a murine immunoglobulin.

In one embodiment, at least one of the evaluated physically associated compounds is non-covalently associated with the immunoglobulin. Such compounds may be directly or indirectly physically associated with the immunoglobulin. An indirect interaction may be bridged by one or more compounds, at least one of which is directly associated with the immunoglobulin. In one embodiment, an associated compound is an antigen recognized by the immunoglobulin. For example, an antigen that is a component of a pathogen, e.g., a virus or bacterium, e.g., a replicable virus or live bacterium.

The method can include further including separating the at least one non-covalently associated compounds from the immunoglobulin, e.g., prior to the evaluating. The separating from the immunoglobulin can include covalently attaching the immunoglobulin to an insoluble support, e.g., a matrix, a particle, or a surface.

The separating can include denaturing the immunoglobulin, e.g., using a chaotrope, an organic solvent, high or low pH, or heat. In another embodiment, wherein at least one of the evaluated associated compounds is protease resistant (e.g., includes a non-proteinaceous component), the separating can include degrading the immuno-globulin.

The evaluating can include one or more of: gel electrophoresis, mass spectroscopy, chromatography, protein sequencing, detecting a label (e.g., a radioactive, fluorescent, enzymatic, or chemical label), detecting a given compound using an affinity reagent specific for the given compound, or another method described herein. The affinity reagent may be an antibody. For example, the detecting can include performing an immuno-blot or an immuno-precipitation.

The evaluating can include culturing a pathogen (e.g., virus or bacterium) that is associated with the immunoglobulin.

The subject of the evaluating can include a proteinaceous or a non-proteinaceous chemical compound. For example, the subject can include a peptide, a polypeptide, a protein complex, or a drug. In one embodiment, the compound is other than an antigen, e.g., the compound is associated with the immunoglobulin by interactions outside the CDR region.

In one embodiment, the evaluating includes eluting an associated compound from the immunoglobulin by competition using a synthetic affinity ligand specific for an epitope of the immunoglobulin or an antigen. The natural compound can include a negatively charged aromatic group having a molecular weight of less than 500 Daltons.

In one embodiment, the immunoglobulin is an artificial variant of a naturally-occurring immunoglobulin. For example, the immunoglobulin can be fused to a heterologous polypeptide or covalently coupled to a therapeutic agent (e.g., a cytotoxic drug).

The method can further include digitally recording information that (i) indicates the presences or absence of a given compound among the evaluated one or more physically associated compounds, or (ii) describes the one or more physically associated compounds.

In one embodiment, the method further includes providing a second sample, and evaluating one or more of the physically associated compounds in the second sample. The method can further include comparing results of evaluating the one or more of the physically associated compounds for the first sample to the second sample.

In one embodiment, the sample is obtained from a subject, e.g., a human, e.g., a patient. The sample may include blood or serum. In another example, the sample is obtained from a biopsy, e.g., obtained from a tumor, a region adjacent to a tumor, or a lymph node. The subject may be treated with a therapeutic composition prior to obtaining the sample.

In one embodiment, one or more of the evaluated physically associated compounds is an endogenous compound. In another embodiment, one or more of the evaluated physically associated compounds is a component of the therapeutic composition.

In still another aspect, the invention features a method that includes: providing a complex including a serum albumin and an associated compound; evaluating binding of a non-antibody ligand (e.g., a peptide ligand described herein) to the complex, wherein the non-antibody ligand binds to serum albumin, e.g., with an affinity of less than 5, 3, 2, 1, 0.5, or 0.1 μM and binding of the non-antibody ligand to the complex indicates that the associated compound does not bind an epitope that overlaps the epitope bound by the non-antibody ligand. The method can be used to map a physical interaction between serum albumin and an associated compound. The method can also be varied, e.g., by first binding the ligand and then binding the associated compound.

The method can further include: evaluating binding of a ligand to the complex, wherein the second ligand binds to serum albumin, e.g., with an affinity of less than 5, 3, 2, 1, 0.5, or 0.1 μM. For example, the second ligand is other than an antibody, e.g., a peptide ligand. In one embodiment, one of the first and second non-antibody ligand binds is prevented from binding to the complex. For example, the associated compound sterically hinders binding of the non-antibody ligand to serum albumin or occludes the binding site of the non-antibody ligand for serum albumin. The ligand can be a ligand described herein. The method can also be varied, e.g., by first binding the ligands and then binding the associated compound.

In a related aspect, the invention features a method that includes: providing a complex including a serum albumin and a non-antibody ligand, and evaluating binding of a given compound to the complex. The given compound can be a compound known to bind to serum albumin or a compound isolated from a sample in association with serum albumin, e.g., by a method described herein. The method can be used to map a physical interaction between serum albumin and a given compound.

The method can be repeated for a second ligand. In one embodiment, the second ligand does not include an antigen binding immunoglobulin domain.

The method can be repeated for a second given compound.

The method can include other features described herein.

In another aspect, the invention features a database, including (i) data describing compounds associated with a serum protein in a sample; and (ii) data indicating information about the sample, wherein instances of (i) are linked to instances of (ii). The data can be obtained from results of a method described herein.

In another aspect, the invention features a method (e.g., a machine-based method) that includes: receiving information about compounds associated with a serum protein in a given sample; comparing the information to a database that includes information about compounds associated with the serum protein in a plurality of reference samples to locate information about a compound or a sample indicated by the received information; and providing the located information or a reference to the located information to a user. The method can include other features described herein.

In still another aspect, the invention features a machine-readable medium having encoded thereon information representing a separation process that separates compounds in a composition described herein and/or information representing a characteristic (e.g., physical characteristic, chemical structure, and so forth) of a compound associated with a serum protein (e.g., a serum albumin or an immunoglobulin).

The invention also features an image of a two-dimensional gel that separates a composition described herein. Also featured is a database including a plurality of images, each image corresponding to a two-dimensional gel separation of a composition described herein.

Also featured is a machine-readable medium having encoded thereon information representing characteristics of a plurality of compounds detectable in a composition described herein. Exemplary characteristics include molecular weight, isoelectric point, sequence, chemical composition, abundance, proteolytic fragment profile, and so forth.

In another aspect, the invention features a method that includes a sample that includes (i) a serum protein, (ii) one or more compound physically associated with the serum protein and (iii) a serum protein-binding agent; allowing the serum protein-binding agent to bind to the serum protein to form a complex; separating said complex from one or more components of the sample; and evaluating one or more of the physically associated compounds. Examples of serum proteins include serum albumin, antibodies (e.g., IgG, IgM, and so forth), transferrin, α-macroglobulins, ferritin, apolipoproteins, transthyretin, protease inhibitors, retinol binding protein, thiostatin, α-fetoprotein, vitamin-D binding protein, and afamin. The method can include other features, e.g., as described above and elsewhere herein.

In one embodiment, the method includes obtaining the sample from a subject. For example, the subject may have a metabolic disorder, and the serum protein is a non-albumin carrier protein for one or more metabolites.

In another related aspect, the invention features a method that includes: providing a sample that comprises a serum albumin having one or more compounds physically associated with the serum albumin; isolating the serum albumin and one or more compounds physically associated with the serum albumin from the sample using an affinity reagent that binds the serum albumin; and detecting the one or more physically associated compounds. The method can be used for detecting a serum albumin-associated compound.

In one embodiment, the affinity reagent includes a proteinaceous ligand that is does not have an antigen-binding immunoglobulin domain. For example, the proteinaceous ligand is a peptide ligand that binds serum albumin, e.g., with an affinity of less than 5, 4, 2, 1, 0.5, or 0.1 μM. The affinity reagent can include one or more peptide ligands described herein, e.g., DX-236 and DX-321. In one embodiment, the affinity reagent includes two ligands that bind different epitopes. The method can include other features, e.g., as described above and elsewhere herein.

In still other aspects, the invention features a method that includes administering a composition that comprises a compound to a subject and determining association of the compound with a serum protein from the subject. The determining can include covalently or non-covalently binding the serum protein (e.g., a serum albumin) from the subject to an affinity reagent, e.g., a ligand described herein. The method can include one or more other features described herein.

In another aspect, the invention features a method that includes contacting a serum albumin to a given compound; binding the serum albumin to an affinity reagent described herein; and determining association of the given compound to the serum albumin. The method can include one or more other features described herein.

The method can be used for discovering associations between serum proteins and natural compounds, and, similarly, associations between serum proteins and non-natural compounds, such as pharmaceuticals. The method can also be used to characterize a subject (e.g., a human patient or an animal) by the profile of compounds associated with a given serum protein (e.g., serum albumin).

In some embodiments, peptide ligands are used as affinity reagents to bind a serum protein. Peptide ligands offer several advantages. For example, the mass per binding site is low, e.g., such low molecular weight peptide domains can show higher bind activity per gram than larger proteins such as antibodies. The possibility of non-specific binding is reduced because there is only a small surface available. Peptides can be engineered to have unique tethering sites such as N-terminal Ser or Thr residues or terminal single or multiple lysine segments, e.g., by chemical synthetic methods. (N-terminal Ser or Thr can be specifically oxidized to aldehydes that can be joined to other molecules with high specificity.) Further, as used in some embodiments, a constrained peptide structure is likely to retain its functionality in a variety of contexts.

The invention also features isolated preparations of an endogenous compound associated with a serum albumin. The preparations can be isolated by a method described herein. The preparation can include a single species that also be at least 50, 60, 70, 80, 90, or 95% pure (weight/volume). The species can have an isoelectric point and molecular weight according to a species isolated in FIG. 1.

An example of a non-naturally occurring, serum albumin-binding agent is a polypeptide comprising the amino acid sequence of:

Cys-Xaa₁-Xaa₂-Xaa₃-Xaa₄-Cys (SEQ ID NO: 1),

wherein Xaa₁ is Asp, Asn, Ser, Thr, or Trp; Xaa₂ is Asn, Gln, His, lie, Leu, or Lys; Xaa₃ is Ala, Asp, Phe, Trp, or Tyr; and Xaa₄ is Asp, Gly, Leu, Phe, Ser, or Thr.

Another example of a non-naturally occurring, serum albumin-binding agent is a polypeptide comprising the amino acid sequence of:

Xaa₁-Xaa₂-Xaa₃-Cys-Xaa₄-Xaa₅-Xaa₆-Xaa₇-Cys-Xaa₈-Xaa₉-Xaa₁₀ (SEQ ID NO: 2),

wherein Xaa₁ is Asn, His, Leu, Phe, Trp, or Val; Xaa₂ is Ala, Glu, His, Lys, Trp, or Val; Xaa₃ is Asp, Gly, Ile, His, Ser, Trp, or Val; Xaa₄ is Asp, Asn, Ser, Thr, or Trp; Xaa₅ is Asn, Gln, His, Ile, Leu, or Lys; Xaa₆ is Ala, Asp, Phe, Trp, or Tyr; Xaa₇ is Asp, Gly, Leu, Phe, Ser, or Thr; Xaa₈ is Glu, Ile, Leu, Met, Ser, or Val; Xaa₉ is Asn, Asp, Gln, Gly, Met, Ser, or Trp; and Xaa₁₀ is Ala, Asn, Asp, Pro, Tyr, or Val.

Another example of a non-naturally occurring, serum albumin-binding agent is a polypeptide comprising the amino acid sequence of:

Ala-Glu-Gly-Thr-Gly-Ser-Xaa₁-Xaa₂-Xaa₃-Cys-Xaa₄-Xaa₅-Xaa₆-Xaa₇-Cys-Xaa₈-Xaa₉-Xaa₁₀-Ala-Pro-Glu (SEQ ID NO: 3),

wherein Xaa₁ is Asn, His, Leu, Phe, Trp, or Val; Xaa₂ is Ala, Glu, His, Lys, Trp, or Val; Xaa₃ is Asp, Gly, Ile, His, Ser, Trp, or Val; Xaa₄ is Asp, Asn, Ser, Thr, or Trp; Xaa₅ is Asn, Gln, His, Ile, Leu, or Lys; Xaa₆ is Ala, Asp, Phe, Trp, or Tyr; Xaa₇ is Asp, Gly, Leu, Phe, Ser, or Thr; Xaa₈ is Glu, Ile, Leu, Met, Ser, or Val; Xaa₉ is Asn, Asp, Gln, Gly, Met, Ser, or Trp; and Xaa₁₀ is Ala, Asn, Asp, Pro, Tyr, or Val.

Another example of a non-naturally occurring, serum albumin-binding agent is a polypeptide comprising the amino acid sequence of:

Cys-Xaa₁-Xaa₂-Xaa₃-Xaa₄-Xaa₅-Xaa₆-Cys (SEQ ID NO: 431)

wherein Xaa₁ is Ala, Leu, His, Met, Phe, Ser, or Thr; Xaa₂ is Ile, Phe, Pro, Ser, Trp, or Tyr; Xaa₃ is Asp, Gln, Glu, Lys, Pro, Trp, or Tyr; Xaa₄ is Asp, Gln, Gly, Leu, Pro, or Trp; Xaa₅ is Asp, Ile, Leu, Lys, Met, Pro, Trp, or Tyr; and Xaa₆ is Gln, Gly, Ile, Phe, Thr, Trp, or Val.

Another example of a non-naturally occurring, serum albumin-binding agent is a polypeptide comprising the amino acid sequence of:

Xaa₁-Xaa₂-Xaa₃-Cys-Xaa₄-Xaas-Xaa₆-Xaa₇-Xaa₈-Xaa₉-Cys-Xaa₁₀-Xaa₁₁-Xaa₁₂ (SEQ ID NO: 99), wherein Xaa₁ is Ala, Gln, Leu, Lys, Phe, Trp, or Tyr;Xaa₂ is Asn, Gln, Glu, Ile, Thr, or Trp; Xaa₃ is Asn, Gly, Phe, Thr, Trp, or Tyr; Xaa₄ is Ala, Leu, His, Met, Phe, Ser, or Thr; Xaa₅ is Ile, Phe, Pro, Ser, Trp, or Tyr; Xaa₆ is Asp, Gln, Glu, Lys, Pro, Trp, or Tyr; Xaa₇ is Asp, Gln, Gly, Leu, Pro, or Trp; Xaa₈ is Asp, Ile, Leu, Lys, Met, Pro, Trp, or Tyr; Xaa₉ is Gln, Gly, Ile, Phe, Thr, Trp, or Val; Xaa₁₀ is Asp, Glu, Gly, Leu, Lys, Pro, or Ser; Xaa₁₁ is Glu, His, Ile, Leu, Lys, Ser, Trp, or Val; and Xaa₁₂ is Ala, Asn, His, Ile, Met, Phe, Pro, or Ser.

Another example of a non-naturally occurring, serum albumin-binding agent is a polypeptide comprising the amino acid sequence of:

Ala-Gly-Xaa₁-Xaa₂-Xaa₃-Cys-Xaa₄-Xaa₅-Xaa₆-Xaa₇-Xaa₈-Xaa₉-Cys-Xaa₁₀-Xaa₁₁-Xaa₁₂-Gly-Thr (SEQ ID NO: 100),

wherein Xaa₁ is Ala, Gln, Leu, Lys, Phe, Trp, or Tyr; Xaa₂ is Asn, Gln, Glu, Ile, Thr, or Trp; Xaa₃ is Asn, Gly, Phe, Thr, Trp, or Tyr; Xaa₄ is Ala, Leu, His, Met, Phe, Ser, or Thr; Xaa₅ is Ile, Phe, Pro, Ser, Trp, or Tyr; Xaa₆ is Asp, Gln, Glu, Lys, Pro, Trp, or Tyr; Xaa₇ is Asp, Gln, Gly, Leu, Pro, or Trp; Xaa₈ is Asp, Ile, Leu, Lys, Met, Pro, Trp, or Tyr; Xaa₉ is Gln, Gly, Ile, Phe, Thr, Trp, or Val; Xaa₁₀ is Asp, Glu, Gly, Leu, Lys, Pro, or Ser; Xaa₁₁ is Glu, His, Ile, Leu, Lys, Ser, Trp, or Val; and Xaa₁₂ is Ala, Asn, His, Ile, Met, Phe, Pro, or Ser.

Another example of a non-naturally occurring, serum albumin-binding agent is a polypeptide comprising the amino acid sequence of:

Cys-Xaa₁-Xaa₂-Xaa₃-Xaa₄-Xaa₅-Xaa₆-Xaa₇-Xaa₈-Cys (SEQ ID NO: 101),

wherein Xaa₁ is Gln, Glu, Phe, or Met; Xaa₂ is Asp, Pro, or Thr; Xaa₃ is Ile, Ser, or Trp; Xaa₄ is His, Met, Phe or Pro; Xaa₅ is Asn, Leu, or Thr; Xaa₆ is Arg, Asn, His, or Thr; Xaa₇ is Arg, Met, Phe, or Tyr; and Xaa₈ is Asp, Gly, Phe, or Trp.

Another example of a non-naturally occurring, serum albumin-binding agent is a polypeptide comprising the amino acid sequence of:

Xaa₁-Xaa₂-Xaa₃-Cys-Xaa₄-Xaa₅-Xaa₆-Xaa₇-Xaa₈-Xaa₉-Xaa₁₀-Xaa₁₁-Cys-Xaa₁₂-Xaa₁₃-Xaa₁₄ (SEQ ID NO: 102),

wherein Xaa₁ is Arg, Phe, or Tyr; Xaa₂ is Arg, Leu, Ser, or Trp; Xaa₃ is Asn, Asp, Phe, or Tyr; Xaa₄ is Gln, Glu, Phe, or Met; Xaa₅ is Asp, Pro, or Thr; Xaa₆ is Ile, Ser, or Trp; Xaa₇ is His, Met, Phe or Pro; Xaa₈ is Asn, Leu, or Thr; Xaa₉ is Arg, Asn, His, or Thr; Xaa₁₀ is Arg, Met, Phe, or Tyr; Xaa₁₁ is Asp, Gly, Phe, or Trp; Xaa₁₂ is Ala, Asn, or Asp; Xaa₁₃ is Arg, Phe, Pro, or Tyr; and Xaa₁₄ is Arg, His, Phe, or Ser.

Another example of a non-naturally occurring, serum albumin-binding agent is a polypeptide comprising the amino acid sequence of:

Gly-Ser-Xaa₁-Xaa₂-Xaa₃-Cys-Xaa₄-Xaa₅-Xaa₆-Xaa₇-Xaa₈-Xaa₉-Xaa₁₀-Xaa₁₁-Cys-Xaa₁₂-Xaa₁₃-Xaa₁₄-Ala-Pro (SEQ ID NO: 103),

wherein Xaa₁ is Arg, Phe, or Tyr; Xaa₂ is Arg, Leu, Ser, or Trp; Xaa₃ is Asn, Asp, Phe, or Tyr; Xaa₄ is Gln, Glu, Phe, or Met; Xaa₅ is Asp, Pro, or Thr; Xaa₆ is Ile, Ser, or Trp; Xaa₇ is His, Met, Phe or Pro; Xaa₈ is Asn, Leu, or Thr; Xaa₉ is Arg, Asn, His, or Thr; Xaa₁₀ is Arg, Met, Phe, or Tyr; Xaa₁₁ is Asp, Gly, Phe, or Trp; Xaa₁₂ is Ala, Asn, or Asp; Xaa₁₃ is Arg, Phe, Pro, or Tyr; and Xaa₁₄ is Arg, His, Phe, or Ser.

Another example of a non-naturally occurring, serum albumin-binding agent is a polypeptide comprising the amino acid sequence of:

Cys-Xaa₁-Xaa₂-Xaa₃-Xaa₄-Xaa₅-Xaa₆-Xaa₇-Xaa₈-Xaa₉-Xaa₁₀-Cys (SEQ ID NO: 4),

wherein Xaa₁ is Ala, Asn, Asp, Gln, Glu, Gly, Ile, Leu, Lys, Phe, Pro, Ser, Thr, Trp, Tyr, or Val; Xaa₂ is Ala, Arg, Asp, Glu, Gly, His, Ile, Leu, Lys, Met, Phe, Ser, Thr, Trp, Tyr, or Val; Xaa₃ is Ala, Arg, Asp, Gln, Glu, Gly, Ile, Leu, Lys, Met, Pro, Ser, Thr, Trp, Tyr, or Val; Xaa₄ is Ala, Arg, Asn, Asp, Ile, Leu, Phe, Pro, Ser, Trp, or Tyr; Xaa₅ is Ala, Asp, Glu, Gly, Ile, Met, Phe, Pro, Thr, Trp, or Tyr; Xaa₆ is Ala, Arg, Asn, Asp, Gln, Glu, His, Ile, Leu, Lys, Phe, Ser, Thr, Trp, or Tyr; Xaa₇ is Ala, Arg, Asp, Glu, Gly, His, Met, Phe, Pro, Ser, Thr, or Trp; Xaa₈ is Ala, Arg, Asp, Gln, Glu, His, Ile, Leu, Met, Phe, Pro, Ser, Thr, Trp, or Val; Xaa₉ is Ala, Arg, Asp, Gln, Glu, His, Ile, Leu, Lys, Met, Phe, Pro, Ser, Thr, Trp, Tyr, or Val; Xaa₁₀ is Ala, Asp, Gln, Glu, Gly, His, Ile, Leu, Lys, Phe, Pro, Ser, Thr, Trp, Tyr, or Val.

Another example of a non-naturally occurring, serum albumin-binding agent is a polypeptide comprising the amino acid sequence of:

Xaa₁-Xaa₂-Xaa₃-Cys-Xaa₄-Xaa₅-Xaa₆-Xaa₇-Xaa₈-Xaa₉-Xaa₁₀-Xaa₁₁-Xaa₁₂-Xaa₁₃-Cys-Xaa₁₄-Xaa₁₅-Xaa₁₆ (SEQ ID NO: 5),

wherein Xaa₁ is Ala, Arg, Asp, Asn, Gly, His, Leu, Phe, Pro, Ser, Trp, Tyr; Xaa₂ is Ala, Arg, Asp, Asn, Gly, His, Phe, Pro, Ser, or Trp; Xaa₃ is Ala, Asn, Asp, Gln, Glu, Gly, His, Leu, Met, Phe, Ser, Thr, Trp, Tyr, or Val; Xaa₄ is Ala, Asn, Asp, Gln, Glu, Gly, Ile, Leu, Lys, Phe, Pro, Ser, Thr, Trp, Tyr, or Val; Xaa₅ is Ala, Arg, Asp, Glu, Gly, His, Ile, Leu, Lys, Met, Phe, Ser, Thr, Trp, Tyr, or Val; Xaa₆ is Ala, Arg, Asp, Gln, Glu, Gly, Ile, Leu, Lys, Met, Pro, Ser, Thr, Trp, Tyr, or Val; Xaa₇ is Ala, Arg, Asn, Asp, Ile, Leu, Phe, Pro, Ser, Trp, or Tyr; Xaa₈ is Ala, Asp, Glu, Gly, Ile, Met, Phe, Pro, Thr, Trp, or Tyr; Xaa₉ is Ala, Arg, Asn, Asp, Gln, Glu, His, Ile, Leu, Lys, Phe, Ser, Thr, Trp, or Tyr; Xaa₁₀ is Ala, Arg, Asp, Glu, Gly, His, Met, Phe, Pro, Ser, Thr, or Trp; Xaa₁₁ is Ala, Arg, Asp, Gln, Glu, His, Ile, Leu, Met, Phe, Pro, Ser, Thr, Trp, or Val; Xaa₁₂ is Ala, Arg, Asp, Gln, Glu, His, Ile, Leu, Lys, Met, Phe, Pro, Ser, Thr, Trp, Tyr, or Val; Xaa₁₃ is Ala, Asp, Gln, Glu, Gly, His, Ile, Leu, Lys, Phe, Pro, Ser, Thr, Trp, Tyr, or Val; Xaa₁₄ is Ala, Arg, Asn, Asp, Glu, Gly, His, Ile, Leu, Lys, Met, Pro, Ser, Thr, Trp, or Tyr; Xaa₁₅ is Ala, Arg, Asn, Asp, Gly, His, Leu, Phe, Pro, Ser, Trp, or Tyr; and Xaa₁₆ is Ala, Asn, Asp, Gly, His, Leu, Phe, Pro, Ser, Trp, or Tyr.

Further examples of serum albumin-binding ligands that have the structure of SEQ ID NO: 5, above, include polypeptides comprising the amino acid sequence (A) or (B):

(A) Xaa₁-Arg-Xaa₂-Cys-Xaa₃-Thr-Xaa₄-Xaa₅-Pro-Xaa₆-Xaa₇-Xaa₈-Xaa₉-Xaa₁₀-Cys-Xaa₁₁-Xaa₁₂-Xaa₁₃ (SEQ ID NO: 425),

wherein Xaa₁ is Asn, Leu, or Phe, preferably Leu; Xaa₂ is Ala, Asn, Asp, Gln, Glu, Gly, His, Leu, Met, Phe, Ser, Thr, Trp, Tyr, or Val; Xaa₃ is Ala, Asn, Asp, Gln, Glu, Gly, Ile, Leu, Lys, Phe, Pro, Ser, Thr, Trp, Tyr, or Val; Xaa₄ is Ala, Arg, Asp, Gln, Glu, Gly, Ile, Leu, Lys, Met, Pro, Ser, Thr, Trp, Tyr, or Val; Xaa₅ is Phe, Trp, or Tyr, preferably Trp; Xaa₆ is His or Phe, preferably Phe; Xaa₇ is Asp, Glu, or Thr; Xaa₈ is Ala, Arg, Asp, Gln, Glu, His, Ile, Leu, Met, Phe, Pro, Ser, Thr, Trp, or Val; Xaa₉ is Ala, Arg, Asp, Gln, Glu, His, Ile, Leu, Lys, Met, Phe, Pro, Ser, Thr, Trp, Tyr, or Val; Xaa₁₀ is Ala, Asp, Gln, Glu, Gly, His, Ile, Leu, Lys, Phe, Pro, Ser, Thr, Trp, Tyr, or Val; Xaa₁₁ is Pro or Ser; Xaa₁₂ is Asn or Pro; and Xaa₁₃ is Asn or Pro; or

(B) Xaa₁-Xaa₂-Xaa₃-Cys-Ile-Thr-Xaa₄-Pro-Phe-Xaa₅-Xaa₆-Xaa₇-Xaa₈-Xaa₉-Cys-Xaa₁₀-Asn-Xaa₁₁ (SEQ ID NO: 426),

wherein Xaa₁ is Ala, Arg, Asp, Asn, Gly, His, Leu, Phe, Pro, Ser, Trp, Tyr; Xaa₂ is Ala, Arg, Asp, Asn, Gly, His, Phe, Pro, Ser, or Trp; Xaa₃ is Glu, Leu, or Met, preferably Met; Xaa₄ is Trp or Tyr, preferably Trp; Xaa₅ is Gln, Glu, or Lys; Xaa₆ is Ala, Arg, Asp, Glu, Gly, His, Met, Phe, Pro, Ser, Thr, or Trp; Xaa₇ is Met, Pro, or Ser, preferably Pro; Xaa₈ is Ala, Arg, Asp, Gln, Glu, His, Ile, Leu, Lys, Met, Phe, Pro, Ser, Thr, Trp, Tyr, or Val; Xaa₉ is His or Pro, preferably Pro; Xaa₁₀ is Ala, Arg, Asn, Asp, Glu, Gly, His, Ile, Leu, Lys, Met, Pro, Ser, Thr, Trp, or Tyr; and Xaa₁₁ is Ala, Asn, Asp, Gly, His, Leu, Phe, Pro, Ser, Trp, or Tyr.

Still other non-naturally occurring, serum albumin-binding ligands include a polypeptide comprising the amino acid sequence of:

Ala-Glu-Gly-Thr-Gly-Xaa₀-Xaa₁-Xaa₂-Xaa₃-Cys-Xaa₄-Xaa₅-Xaa₆-Xaa₇-Xaa₈-Xaa₉-Xaa₁₀-Xaa₁₁-Xaa₁₂-Xaa₁₃-Cys-Xaa₁₄-Xaa₁₅-Xaa₁₆-Xaa₁₇-Pro-Glu (SEQ ID NO: 6),

wherein Xaa₀ is Ala or Asp; Xaa₁ is Ala, Arg, Asp, Asn, Gly, His, Leu, Phe, Pro, Ser, Trp, Tyr; Xaa₂ is Ala, Arg, Asp, Asn, Gly, His, Phe, Pro, Ser, or Trp; Xaa₃ is Ala, Asn, Asp, Gln, Glu, Gly, His, Leu, Met, Phe, Ser, Thr, Trp, Tyr, or Val; Xaa₄ is Ala, Asn, Asp, Gln, Glu, Gly, Ile, Leu, Lys, Phe, Pro, Ser, Thr, Trp, Tyr, or Val; Xaa₅ is Ala, Arg, Asp, Glu, Gly, His, Ile, Leu, Lys, Met, Phe, Ser, Thr, Trp, Tyr, or Val; Xaa₆ is Ala, Arg, Asp, Gln, Glu, Gly, Ile, Leu, Lys, Met, Pro, Ser, Thr, Trp, Tyr, or Val; Xaa₇ is Ala, Arg, Asn, Asp, Ile, Leu, Phe, Pro, Ser, Trp, or Tyr; Xaa₈ is Ala, Asp, Glu, Gly, Ile, Met, Phe, Pro, Thr, Trp, or Tyr; Xaa₉ is Ala, Arg, Asn, Asp, Gln, Glu, His, Ile, Leu, Lys, Phe, Ser, Thr, Trp, or Tyr; Xaa₁₀ is Ala, Arg, Asp, Glu, Gly, His, Met, Phe, Pro, Ser, Thr, or Trp; Xaa₁₁ is Ala, Arg, Asp, Gln, Glu, His, Ile, Leu, Met, Phe, Pro, Ser, Thr, Trp, or Val; Xaa₁₂ is Ala, Arg, Asp, Gln, Glu, His, Ile, Leu, Lys, Met, Phe, Pro, Ser, Thr, Trp, Tyr, or Val; Xaa₁₃ is Ala, Asp, Gln, Glu, Gly, His, Ile, Leu, Lys, Phe, Pro, Ser, Thr, Trp, Tyr, or Val; Xaa₁₄ is Ala, Arg, Asn, Asp, Glu, Gly, His, Ile, Leu, Lys, Met, Pro, Ser, Thr, Trp, or Tyr; Xaa₁₅ is Ala, Arg, Asn, Asp, Gly, His, Leu, Phe, Pro, Ser, Trp, or Tyr; Xaa₁₆ is Ala, Asn, Asp, Gly, His, Leu, Phe, Pro, Ser, Trp, or Tyr; and Xaa₁₇ is Ala or Asp.

In a further embodiment, the invention provides a non-naturally occurring, serum albumin-binding agent comprising a linear polypeptide comprising an amino acid sequence selected from the group consisting of:

(SEQ ID NO:104)

P T V V Q P K F H A F T H E D L L W I F,

(SEQ ID NO:105)

L K S Q M V H A L P A A S L H D Q H E L, and

(SEQ ID NO:106)

S Q V Q G T P D L Q F T V R D F I Y M F.

Examples of serum albumin-binding agents include polypeptides that include an amino acid sequence selected from the group consisting of (depicted using the standard single letter abbreviations for the twenty common L-amino acids):

| | |
|---|---|
| C T I F L C, | (SEQ ID NO:7) |
| C E G K D M I D W V Y C, | (SEQ ID NO:8) |
| C D R I A W Y P Q H L C, | (SEQ ID NO:9) |
| C D R I A W Y P Q H A C, | (SEQ ID NO:41) |
| C D R I A W Y P Q A L C, | (SEQ ID NO:42) |
| C D R I A W Y P A H L C, | (SEQ ID NO:43) |
| C D R I A W Y A Q H L C, | (SEQ ID NO:44) |
| C D R I A W A P Q H L C, | (SEQ ID NO:45) |
| C D R I A A Y P Q H L C, | (SEQ ID NO:46) |
| C D R A A W Y P Q H L C, | (SEQ ID NO:47) |
| C D A I A W Y P Q H L C, | (SEQ ID NO:48) |
| C A R I A W Y P Q H L C, | (SEQ ID NO:49) |
| C E P W M L R F G C, | (SEQ ID NO:10) |
| C D Q W F C, | (SEQ ID NO:11) |
| C N N A L C, | (SEQ ID NO:12) |
| C D H F F C, | (SEQ ID NO:13) |
| C W H F S C, | (SEQ ID NO:14) |
| C V T R W A N R D Q Q C, | (SEQ ID NO:15) |
| C V T D W A N R H Q H C, | (SEQ ID NO:16) |
| C V K D W A N R R R G C, | (SEQ ID NO:17) |
| C K F S W I R S P A F C, | (SEQ ID NO:18) |
| C Q T T W P F T M M Q C, | (SEQ ID NO:107) |
| C V T M W P F E Q I F C, | (SEQ ID NO:108) |
| C F T Y Y P F T T F S C, | (SEQ ID NO:109) |
| C W T K F P F D L V W C, | (SEQ ID NO:110) |
| C V S Y W P H F V P V C, | (SEQ ID NO:111) |
| C Y I S F P F D Q M Y C, | (SEQ ID NO:112) |
| C S V Q Y P F E V V V C, | (SEQ ID NO:113) |
| C W T Q Y P F D H S T C, | (SEQ IID NO:114) |
| C I T W P F K R P W P C, | (SEQ ID NO:115) |
| C I S W P F E M P F H C, | (SEQ ID NO:116) |
| C I T W P F K R P W P C, | (SEQ ID NO:117) |
| C I T Y P F H E M F P C, | (SEQ ID NO: 118) |
| C I T W P F Q T S Y P C, | (SEQ ID NO:119) |
| C K F S W I R S P A F C, | (SEQ ID NO:120) |
| C W I V D E D G T K W C, | (SEQ ID NO:121) |
| C D S A Y W Q E I P A C, | (SEQ ID NO:122) |
| C L W D P M L C, | (SEQ ID NO:123) |
| C E H P Y W T E V D K C, | (SEQ ID NO:124) |
| C D T P Y W R D L W Q C, | (SEQ ID NO:125) |
| C Q L P Y M S T P E F C, | (SEQ ID NO:126) |
| C G R G F D K E S I Y C, | (SEQ ID NO:127) |
| C V T Y I G T W E T V C, | (SEQ ID NO:128) |
| C T D T N W S W M F D C, | (SEQ ID NO:129) |
| C T L E I G T W F V F C, | (SEQ ID NO:130) |
| C K I A L F Q H F E V C, | (SEQ ID NO:131) |
| C I K L Y G L G H M Y C, | (SEQ ID NO:132) |
| C E M Q S I I P W W E C, | (SEQ ID NO:133) |
| C V E K Y Y W D V L I C, | (SEQ ID NO:134) |
| C P H G R Y S M F P C, | (SEQ ID NO:135) |
| C N V R W T D T P Y W C, | (SEQ ID NO:136) |
| C T Y D P I A D L L F C, | (SEQ ID NO:137) |
| C M D W P N H R D C, | (SEQ ID NO:138) |
| C F P I H L T M F C, | (SEQ ID NO:139) |
| C Q T S F T N Y W C, | (SEQ ID NO:140) |
| C M E F G P D D C, | (SEQ ID NO:141) |
| C S W D P I F C, | (SEQ ID NO: 142) |
| C A W D P L V C, | (SEQ ID NO: 143) |
| C H I Y D W F C, | (SEQ ID NO:144) |
| C L W D P M I C, | (SEQ ID NO:145) |
| C S P P G K T C, | (SEQ ID NO:146) |
| C T F W Q Y W C, | (SEQ ID NO:147) |
| C M F E L P F C, | (SEQ ID NO: 148) |
| C F S K P D Q C, | (SEQ ID NO:149) |
| C F Y Q W W G C, | (SEQ ID NO:150) |
| C T W D P I F C, | (SEQ ID NO:151) |
| C W L Y D C, | (SEQ ID NO:152) |
| C D K Y G C, and | (SEQ ID NO:153) |
| C S K D T C. | (SEQ ID NO: 154) |

Additional examples of serum albumin-binding agents are polypeptides that include an amino acid sequence selected from the group consisting of:

| | |
|---|---|
| A D F C E G K D M I D W V Y C R L Y, | (SEQ ID NO:27) |
| F W F C D R I A W Y P Q H L C E F L, | (SEQ ID NO:28) |
| F W F C D R I A W Y P Q H L C E F A, | (SEQ ID NO:50) |
| F W F C D R I A W Y P Q H L C E A L, | (SEQ ID NO:51) |
| F W F C D R I A W Y P Q H L C A F L, | (SEQ ID NO:52) |
| F W F C D R I A W Y P Q H A C E F L, | (SEQ ID NO:53) |
| F W F C D R I A W Y P Q A L C E F L, | (SEQ ID NO:54) |
| F W F C D R I A W Y P A H L C E F L, | (SEQ ID NO:55) |
| F W F C D R I A W Y A Q H L C E F L, | (SEQ ID NO:56) |
| F W F C D R I A W A P Q H L C E F L, | (SEQ ID NO:57) |
| F W F C D R I A A Y P Q H L C E F L, | (SEQ ID NO:58) |
| F W F C D R A A W Y P Q H L C E F L, | (SEQ ID NO:59) |
| F W F C D A I A W Y P Q H L C E F L, | (SEQ ID NO:60) |
| F W F C A R I A W Y P Q H L C E F L, | (SEQ ID NO:61) |
| F W A C D R I A W Y P Q H L C E F L, | (SEQ ID NO:62) |
| F A F C D R I A W Y P Q H L C E F L, | (SEQ ID NO:63) |
| A W F C D R I A W Y P Q H L C E F L, | (SEQ ID NO:64) |
| D W D C V T R W A N R D Q Q C W G P, | (SEQ ID NO:29) |
| D W D C V T R W A N R D Q Q C W A L, | (SEQ ID NO:30) |
| D W D C V T D W A N R H Q H C W A L, | (SEQ ID NO:31) |
| D W Q C V K D W A N R R R G C M A D, | (SEQ ID NO:32) |
| R N M C K F S W I R S P A F C A R A, | (SEQ ID NO:33) |
| L R D C Q T T W P F M M Q C P N N, | (SEQ ID NO:155) |
| N R E C V T M W P F E Q I F C P W P, | (SEQ ID NO:156) |
| L R S C F T Y Y P F T T F S C S P A, | (SEQ ID NO:157) |
| L S H C W T K F P F D L V W C D S P, | (SEQ ID NO:158) |
| L R M C V S Y W P H F V P V C E N P, | (SEQ ID NO:159) |
| L R D C Y I S F P F D Q M Y C S H F, | (SEQ ID NO:160) |
| F R H C S V Q Y P F E V V V C P A N, | (SEQ ID NO:161) |
| L R N C W T Q Y P F D H S T C S P N, | (SEQ ID NO:162) |
| D S M C I T W P F K R P W P C A N, | (SEQ ID NO:163) |
| A F M C I S W P F E M P F H C S P D, | (SEQ ID NO:164) |
| D S M C I T W P F K R P W P C A N P, | (SEQ ID NO:165) |
| W D L C I T Y P F H E M F P C E D G, | (SEQ ID NO:166) |
| G G E C I T W P F Q T S Y P C T N G, | (SEQ ID NO:167) |
| R N M C K F S W I R S P A F C A R A, | (SEQ ID NO:168) |
| F S L C W I V D E D G T K W C L P, | (SEQ ID NO:169) |
| R W F C D S A Y W Q E I P A C A R D, | (SEQ ID NO:170) |
| R W Y C L W D P M L C M S D, | (SEQ ID NO:171) |
| A W Y C E H P Y W T E V D K C H S S, | (SEQ ID NO:172) |
| S D F C D T P Y W R D L W Q C N S P, | (SEQ ID NO:173) |
| L P W C Q L P Y M S T P E F C I R P, | (SEQ ID NO:174) |
| Y H V C G R G F D K E S I Y C K F L, | (SEQ ID NO:175) |
| S F C V T Y I G T W E T V C K R S, | (SEQ ID NO:176) |
| N D G C T D T N W S W M F D C P P L, | (SEQ ID NO:177) |
| W R D C T L E I G T W F V F C K G S, | (SEQ ID NO:178) |
| S P Y C K I A L F Q H F E V C A A D, | (SEQ ID NO:179) |
| R H W C I K L Y G L G H M Y C N R S, | (SEQ ID NO:180) |
| D H A C E M Q S I I P W W E C Y P H, | (SEQ ID NO:181) |
| P R S C V E K Y Y W D V L I C G F F, | (SEQ ID NO:182) |
| F H T C P H G R Y S M F P C D Y W, | (SEQ ID NO:183) |
| H G W C N V R W T D T P Y W C A F S, | (SEQ ID NO:184) |
| Y R V C T Y D P I A D L L F C P F N, | (SEQ ID NO:185) |
| R S F C M D W P N H R D C D Y S, | (SEQ ID NO:186) |
| F W D C F P I H L T M F C D R F, | (SEQ ID NO:187) |
| Y L Y C Q T S F T N Y W C A F H, | (SEQ ID NO:188) |
| G L Y C M E F G P D D C A W H, | (SEQ ID NO:189) |
| K N F C S W D P I F C G I H, | (SEQ ID NO:190) |
| K W Y C A W D P L V C E I F, | (SEQ ID NO:191) |
| W T T C H I Y D W F C S S S, | (SEQ ID NO:192) |
| Q W Y C L W D P M I C G L I, | (SEQ ID NO:193) |
| Q T N C S P P G K T C D K N, | (SEQ ID NO:194) |
| A I C T F W Q Y W C L E P, | (SEQ ID NO:195) |
| F E W C M F E L P F C S W P, | (SEQ ID NO:196) |
| Q E G C F S K P D Q C K V M, | (SEQ ID NO:197) |
| L E Y C F Y Q W W G C P H A, | (SEQ ID NO:198) |
| Y Q F C T W D P I F C G W H, | (SEQ ID NO:199) |
| L W D C W L Y D C E G N, | (SEQ ID NO:200) |
| V H S C D K Y G C V N A, | (SEQ ID NO:201) |
| F E H C S K D T C S G N, | (SEQ ID NO:202) |
| V A W C T I F L C L D V, | (SEQ ID NO:203) |
| F K I C D Q W F C L M P, | (SEQ ID NO:204) |
| H V G C N N A L C M Q Y, | (SEQ ID NO:205) |
| W K V C D H F F C L S P, | (SEQ ID NO:206) |
| N H G C W H F S C I W D, | (SEQ ID NO:207) |
| F R N C E P W M L R F G C N P R, | (SEQ ID NO:208) |
| A D F C E G K D M I D W V Y C R L Y, | (SEQ ID NO:209) |
| F W F C D R I A W Y P Q H L C E F L D, | (SEQ ID NO:210) |
| D W D C V T R W A N R D Q Q C W G P, | (SEQ ID NO:211) |
| D W D C V T R W A N R D Q Q C W A L, | (SEQ ID NO:212) |
| D W D C V T D W A N R H Q H C W A L, | (SEQ ID NO:213) |
| D W Q C V K D W A N R R R G C M A D, | (SEQ ID NO:214) |
| R N M C K F S W I R S P A F C A R A D P, | (SEQ ID NO:215). |

Additional examples of serum albumin-binding agents include polypeptides that comprising an amino acid sequence selected from the group consisting of:

| | |
|---|---|
| A E G T G D A D F C E G K D M I D W V Y C R L Y D P E, | (SEQ ID NO:34) |
| A E G T G D F W F C D R I A W Y P Q H L C E F L D P E, | (SEQ ID NO:35) |
| A E G T G D F W F C D R I A W Y P Q H L C E F L A P E, | (SEQ ID NO:65) |
| A E G T G D F W F C D R I A W Y P Q H L C E F A D P E, | (SEQ ID NO:66) |
| A E G T G D F W F C D R I A W Y P Q H L C E A L D P E, | (SEQ ID NO:67) |
| A E G T G D F W F C D R I A W Y P Q H L C A F L D P E, | (SEQ ID NO:68) |
| A E G T G D F W F C D R I A W Y P Q H A C E F L D P E, | (SEQ ID NO:69) |
| A E G T G D F W F C D R I A W Y P Q A L C E F L D P E, | (SEQ ID NO:70) |
| A E G T G D F W F C D R I A W Y P A H L C E F L D P E, | (SEQ ID NO:71) |
| A E G T G D F W F C D R I A W Y A Q H L C E F L D P E, | (SEQ ID NO:72) |
| A E G T G D F W F C D R I A W A P Q H L C E F L D P E, | (SEQ ID NO:73) |
| A E G T G D F W F C D R I A A Y P Q H L C E F L D P E, | (SEQ ID NO:74) |
| A E G T G D F W F C D R A A W Y P Q H L C E F L D P E, | (SEQ ID NO:75) |
| A E G T G D F W F C D A I A W Y P Q H L C E F L D P E, | (SEQ ID NO:76) |
| A E G T G D F W F C A R I A W Y P Q H L C E F L D P E, | (SEQ ID NO:77) |
| A E G T G D F W A C D R I A W Y P Q H L C E F L D P E, | (SEQ ID NO:78) |
| A E G T G D F A F C D R I A W Y P Q H L C E F L D P E, | (SEQ ID NO:79) |
| A E G T G D A W F C D R I A W Y P Q H L C E F L D P E, | (SEQ ID NO:80) |
| A E G T G A F W F C D R I A W Y P Q H L C E F L D P E, | (SEQ ID NO:81) |
| A E G T G D D W D C V T R W A N R D Q Q C W G P D P E, | (SEQ ID NO:36) |
| A E G T G D D W D C V T R W A N R D Q Q C W A L D P E, | (SEQ ID NO:37) |
| A E G T G D D W D C V T D W A N R H Q H C W A L D P E, | (SEQ ID NO:38) |
| A E G T G D D W Q C V K D W A N R R R G C M A D D P E, and | (SEQ ID NO:39) |
| A E G T G D R N M C K F S W I R S P A F C A R A D P E. | (SEQ ID NO:40) |

Particular examples of a serum albumin-binding agents are polypeptides that include a compound of the formula:

AEGTGDFWFCDRIAWYPQHLCEFLDPEGGGK(SEQ ID NO: 19). This polypeptide is designated DX-236.

DX-236 binds mammalian serum albumins and is useful under appropriate conditions as a “pan mammalian” serum albumin-binding agent. DX-236 variants that include between one and five amino acid changes (substitutions, insertions, or deletions), e.g., between one and three, or one or two,; or between one and six conservative amino acid substitutions, e.g., between one and four, one and three, or one and two; and that bind to a serum albumin can also be used. The following two DX-236 variants can be used: DX-236A which includes the peptide sequence: FWFCDRIAWYPQHLCEFLD (SEQ ID NO: 210) and DX-236B which includes the peptide sequence:

CDRIAWYPQHLC (SEQ ID NO: 9)

DX-236 can also include additional chemical modifications, for example:

Ac-AEGTGDFWFCDRIAWYPQHLCEFLDPEGGGK—NH₂ (SEQ ID NO: 19), wherein Ac indicates an N-terminal acetyl capping group and —NH₂ indicates a C-terminal amide capping group. Examples of DX-236 variants include compounds that include the following sequences:

AEGTGDFWFCDRIAWYPQHLCEFLAPEGGGK—,

AEGTGDFWFCDRIAWYPQHLCEFADPEGGGK—,

AEGTGDFWFCDRIAWYPQHLCEALDPEGGGK—,

AEGTGDFWFCDRIAWYPQHLCAFLDPEGGGK—,

AEGTGDFWFCDRIAWYPQHACEFLDPEGGGK—,

AEGTGDFWFCDRIAWYPQALCEFLDPEGGGK—,

AEGTGDFWFCDRIAWYPAHLCEFLDPEGGGK—,

AEGTGDFWFCDRIAWYAQHLCEFLDPEGGGK—,

AEGTGDFWFCDRIAWAPQHLCEFLDPEGGGK—,

AEGTGDFWFCDRIAAYPQHLCEFLDPEGGGK—,

AEGTGDFWFCDRAAWYPQHLCEFLDPEGGGK—,

AEGTGDFWFCDAIAWYPQHLCEFLDPEGGGK—,

AEGTGDFWFCARIAWYPQHLCEFLDPEGGGK—,

AEGTGDFWACDRIAWYPQHLCEFLDPEGGGK—,

AEGTGDFAFCDRIAWYPQHLCEFLDPEGGGK—,

AEGTGDAWFCDRIAWYPQHLCEFLDPEGGGK—, and

AEGTGAFWFCDRIAWYPQHLCEFLDPEGGGK—,

(SEQ ID NOs: 82 through 98, respectively). The variants can further include an N— or C-terminal modification. Exemplary variants have between one and six, one and five, one and four, or one and three amino acid substitutions, e.g., one or two amino acid substitutions. Other variants include one, two, three, or less than five amino acid insertions, deletions, or substitutions.

Additional serum albumin-binding agents include the following:

GDLRDCQTTWPFTMMQCPNNDPGGGK—,

GDNRECVTMWPFEQIFCPWPDPGGGK—,

GDLRSCFTYYPFTTFSCSPADP GGGK—,

GDDSMCITWPFKRPWPCANDPGGGK—,

GDRNMCKFSWIRSPAFCARADPGGGK—,

GDFSLCWIVDEDGTKWCLPDPGGGK—,

GDRWFCDSAYWQEIPACARDDPGGGK—,

GDSDFCDTPYWRDLWQCNSPDPGGGK—,

GDSFCVTYIGTWETVCKRSDPGGGK—,

GDNDGCTDTNWSWMFDCPPLDPGGGK—,

GDSPYCKIALFQHFEVCAADDPGGGK—,

GDPRSCVEKYYWDVLICGFFDPGGGK—,

GSRSFCMDWPNHRDCDYSAPGGGK—,

AGKWYCAWDPLVCEIFGTGGGK—,

AGWTTCHIYDWFCSSSGTGGGK—,

AGLEYCFYQWWGCPHAGTGGGK—,

AGYQFCTWDPIFCGWHGTGGGK—, and

GSLWDCWLYDCEGNAPGGGK—,

(SEQ ID NOs: 216 through 233, respectively).

Another particular serum albumin-binding agent is a compound that includes: AEGTGDRNMCKFSWIRSPAFCARADPE (SEQ ID NO: 20). This binding moiety is designated polypeptide compound DX-321. Dx-321 can also be modified, e.g., as follows:

Ac-AEGTGDRNMCKFSWIRSPAFCARADPE-X—K—NH₂ (SEQ ID NO: 24), wherein Ac indicates an N-terminal acetyl capping group, X (above) indicates a polypeptide linked 6-aminohexanoic acid group, and —NH₂ indicates a C-terminal amide capping group. DX-321 preferentially binds human serum albumin (HSA) over serum albumins from other species under appropriate conditions. DX-321 is useful as a reagent to specifically detect or isolate HSA. In some embodiments, the compounds do not include the N-terminal acetyl capping group, and may or may not include a C-terminal amide capping group.

DX-321 variants that include between one and five amino acid changes (substitutions, insertions, or deletions), e.g., between one and three, or one or two,; or between one and six conservative amino acid substitutions, e.g., between one and four, one and three, or one and two; and that bind to a serum albumin can also be used. The following DX-321 variants can also be used: DX-321-A which includes the peptide sequence: RNMCKFSWIRSPAFCARA (SEQ ID NO: 430); and DX-321-B which includes the peptide sequence: CKFSWIRSPAFC (SEQ ID NO: 120).

Examples of specific immunoglobulin binding molecules (which bind the Fc region of immunoglobulin) include polypeptides comprising amino acid sequences of the following four general formulae:

Z1-X1-X2-X3-X4-W—C-Z2 (SEQ ID NO: 234); I.

wherein, Z1 is a polypeptide of at least 6 amino acids; X1 is G, H, N, R, or S;

X2 is A, D, E, F, I, M, or S; X3 is A, I, L, M, or V; X4 is I, M, T, or V; Z2 is a polypeptide of at least one amino acid or is absent; and Z1 contains at least one cysteine residue such that formation of a disulfide bond with the invariant cysteine residue forms a cyclic peptide of 12 amino acids.

Z1-X—W-Z2-W-Z3 (SEQ ID NO: 235) II.

wherein, Z1 is a polypeptide of at least one amino acid or is absent;

X is F or Y; Z2 is a tripeptide; and Z3 is a polypeptide of at least one amino acid; and

wherein at least two of the polypeptides Z1, Z2, and Z3 contain a cysteine residue, such that formation of a disulfide bond between such cysteine residues forms a cyclic peptide of 7-12 amino acids.

In the foregoing formula II polypeptides, Z2 can have the formula (IIA):

X1-X2-X3 (IIA),

wherein, X1 is A, C, F, K, P, R, W, or Y; X2 is C, D, E, G, H, K, M, N, Q, R, S, T, V, or Y; and X3 is A, E, F, H, I, K, L, Q, R, S, T, V, or Y; with the proviso that at most one of X1, X2 and X3 can be C. In some implementations, where X2 is C, then X1 is Y. In some implementations, X1 is C.

Z1-W-Z2-W—W-Z3 (SEQ ID NO: 236); III.

wherein, Z1 is a polypeptide of at least one amino acid; Z2 is a tripeptide; and Z3 is a polypeptide of at least one amino acid; wherein at least two of the polypeptides Z 1, Z2, and Z3 contain a cysteine residue, such that formation of a disulfide bond between such cysteine residues forms a cyclic peptide of 8-12 amino acids, with the proviso that where Z1 contains a cysteine, then Z2 does not contain a cysteine, and where Z2 contains a cysteine, it is the middle residue of the tripeptide and Z3 also contains a cysteine.

In some cases, for the polypeptides of formula III, when Z1 and Z3 each contain a cysteine residue, the cysteine of Z1 is adjacent the invariant tryptophan (W), the first amino acid of Z2 is lysine and the second amino acid of Z3 is aspartic acid (D).

Z1-P—X1-W—X2-C—X3-X4-X5 (SEQ ID NO: 237); IV.

wherein, Z1 is a polypeptide of at least one amino acid and includes a cysteine residue; X1 is A, E, R, S, or T; X2 is F, W, or Y; X3 is D, E, L, M, or Q; X4 is H, W, or Y;

X5 is F or Y; and wherein the cysteine residue in Z1 and the cysteine residue between X2 and X3 form a cyclic peptide of 10-12 amino acids.

Examples of immunoglobulin binding polypeptides include polypeptides comprising amino acid sequences selected from the group consisting of:

| | |
|---|---|
| R-R-A-C-S-R-D-W-S-G-A-L-V-W-C-A-G-H | (SEQ ID NO:238) |
| W-G-E-C-T-V-T-S-Y-G-E-L-I-W-C-G-G-L | (SEQ ID NO:239) |
| S-S-A-C-A-F-D-P-M-G-A-V-W-C-T-Y-D | (SEQ ID NO:240) |
| L-L-E-C-A-Y-N-T-S-G-E-L-I-W-C-N-G-S | (SEQ ID NO:241) |
| P-D-D-C-S-I-H-F-S-G-E-L-I-W-C-E-P-L | (SEQ ID NO:242) |
| L-G-E-C-T-V-T-S-Y-G-E-L-I-W-C-G-G-L | (SEQ ID NO:243) |
| W-G-E-C-T-V-T-S-Y-G-E-L-I-W-C-G-G-H | (SEQ ID NO:244) |
| D-H-M-C-V-Y-T-T-W-G-E-L-I-W-C-D-D-H | (SEQ ID NO:245) |
| W-G-E-C-T-V-T-S-Y-G-E-L-I-W-C-G-G-L | (SEQ ID NO:246) |
| C-R-A-C-S-R-D-W-P-G-A-L-V-W-C-A-G-H | (SEQ ID NO:247) |
| R-R-A-C-S-R-D-W-S-G-A-L-V-W-C-A-G-H | (SEQ ID NO:248) |
| L-H-A-C-A-F-D-P-M-G-A-V-I-W-C-T-Y-D | (SEQ ID NO:249) |
| D-H-M-C-V-Y-T-T-W-G-E-L-M-W-C-D-N-H | (SEQ ID NO:250) |
| P-P-T-C-T-W-D-W-Q-G-I-L-V-W-C-S-G-H | (SEQ ID NO:251) |
| S-N-K-C-S-N-T-W-D-G-S-L-I-W-C-S-A-N | (SEQ ID NO:252) |
| F-P-E-C-T-F-D-M-E-G-F-L-I-W-C-S-S-F | (SEQ ID NO:253) |
| H-D-L-C-A-Q-A-P-F-G-D-A-T-W-C-D-L-R | (SEQ ID NO:254) |
| P-N-H-C-S-Y-N-L-K-S-E-L-I-W-C-Q-D-L | (SEQ ID NO:255) |
| P-L-D-C-A-R-D-I-H-N-S-L-I-W-C-S-L-G | (SEQ ID NO:256) |
| G-S-E-C-S-W-T-S-L-N-E-L-I-W-C-A-H-W | (SEQ ID NO:257) |
| W-P-D-C-S-F-T-V-Q-R-D-L-I-W-C-E-A-L | (SEQ ID NO:258) |
| S-H-S-C-A-Y-D-Y-A-H-M-L-V-W-C-T-H-F | (SEQ ID NO:259) |
| D-H-M-C-V-Y-T-T-W-G-E-L-I-W-C-D-N-H | (SEQ ID NO:260) |
| R-P-N-C-T-F-A-A-S-G-E-L-I-W-C-M-H-Y | (SEQ ID NO:261) |
| W-W-G-C-Q-F-D-W-R-G-E-L-V-W-C-P-Y-L | (SEQ ID NO:262) |
| G-G-V-C-S-Y-S-G-M-G-E-I-V-W-C-R-W-F | (SEQ ID NO:263) |
| A-L-M-C-S-H-D-M-W-G-S-L-I-W-C-K-H-F | (SEQ ID NO:264) |
| W-W-N-C-H-N-G-W-T-W-T-G-G-W-C-W-W-F | (SEQ ID NO:265) |
| Y-H-V-C-A-R-D-S-W-D-Q-L-I-W-C-E-A-F | (SEQ ID NO:266) |
| N-Y-W-C-N-F-W-Q-L-P-T-C-D-N-L | (SEQ ID NO:267) |
| Y-W-Y-C-K-W-F-S-E-S-A-S-C-S-S-R | (SEQ ID NO:268) |
| Y-W-Y-C-K-W-F-E-D-K-H-P-C-D-S-S | (SEQ ID NO:269) |
| Y-W-Y-C-S-W-F-P-D-R-P-D-C-P-L-Y | (SEQ ID NO:270) |
| N-Y-W-C-N-V-W-L-L-G-D-V-C-R-S-H | (SEQ ID NO:271) |
| L-Y-W-C-H-V-W-F-G-Q-H-A-W-Q-C-K-Y-P | (SEQ ID NO:272) |
| Y-W-K-C-K-W-M-P-W-M-C-G-F-D | (SEQ ID NO:273) |
| D-D-H-C-Y-W-F-R-E-W-F-N-S-E-C-P-H-G | (SEQ ID NO:274) |
| N-Y-W-C-N-I-W-G-L-H--G-C-N-S-H | (SEQ ID NO:275) |
| Y-W-F-C-Q-W-F-S-Q-N-H-T-C-F-R-D | (SEQ ID NO:276) |
| H-Y-W-C-D-I-W-F-G-A-P-A-C-Q-F-R | (SEQ ID NO:277) |
| S-G-D-C-G-F-W-P-R-I-W-G-L-C-M-D-N | (SEQ ID NO:278) |
| F-W-Y-C-K-W-F-Y-E-D-A-Q-C-S-H-D | (SEQ ID NO:279) |
| Y-Y-W-C-N-Y-W-G-L-C-P-D-Q | (SEQ ID NO:280) |
| S-Y-W-C-K-I-W-D-V-C-P-Q-S | (SEQ ID NO:281) |
| K-Y-W-C-N-L-W-G-V-C-P-A-N | (SEQ ID NO:282) |
| Q-Y-W-C-Y-Q-W-G-L-C-G-A-N | (SEQ ID NO:283) |
| K-Y-W-C-Q-Q-W-G-V-C-N-G-S | (SEQ ID NO:284) |
| K-Y-W-C-V-Q-W-G-V-C-P-E-S | (SEQ ID NO:285) |
| K-Y-W-C-M-Q-W-G-L-C-G-W-E | (SEQ ID NO:286) |
| H-F-W-C-E-V-W-G-L-C-P-S-I | (SEQ ID NO:287) |
| Q-Y-W-C-T-K-W-G-L-C-T-N-V | (SEQ ID NO:288) |
| A-Y-W-C-K-V-W-G-L-C-Q-G-E | (SEQ ID NO:289) |
| K-Y-W-C-N-L-W-G-V-C-P-A-N | (SEQ ID NO:290) |
| Q-Y-W-C-N-V-W-G-V-C-L-P-S | (SEQ ID NO:291) |
| H-Y-W-C-Q-Q-W-G-I-C-E-R-P | (SEQ ID NO:292) |
| R-Y-W-C-N-I-W-D-V-C-P-E-Q | (SEQ ID NO:293) |
| Q-Y-W-C-T-H-W-G-L-C-G-K-Y | (SEQ ID NO:294) |
| T-Y-W-C-T-K-W-G-L-C-P-H-N | (SEQ ID NO:295) |
| F-Y-W-C-G-Q-W-G-L-C-A-P-P | (SEQ ID NO:296) |
| G-Y-W-C-N-V-W-G-L-C-S-T-E | (SEQ ID NO:297) |
| R-Y-W-C-G-V-W-G-V-C-E-I-D | (SEQ ID NO:298) |
| K-F-W-C-T-I-W-G-V-C-H-M-P | (SEQ ID NO:299) |
| H-Y-W-C-Q-Q-W-G-I-C-E-R-P | (SEQ ID NO:300) |
| R-Y-W-C-N-I-W-D-V-C-P-E-Q | (SEQ ID NO:301) |
| F-Y-W-C-S-Q-W-G-L-C-K-Y-D | (SEQ ID NO:302) |
| H-Y-W-C-E-K-W-G-L-C-L-M-S | (SEQ ID NO:303) |
| H-Y-W-C-Q-K-W-G-V-C-P-T-D | (SEQ ID NO:304) |
| H-Y-W-C-S-L-W-G-V-C-D-I-N | (SEQ ID NO:305) |
| R-F-W-C-S-A-W-G-V-C-P-A | (SEQ ID NO:306) |
| S-Y-W-C-K-I-W-D-V-C-P-Q-S | (SEQ ID NO:307) |
| Q-Y-W-C-S-I-W-K-V-C-P-G-R | (SEQ ID NO:308) |
| Y-W-Y-C-E-W-F-G-A-C-I-N-D | (SEQ ID NO:309) |
| E-Y-W-C-K-Y-W-G-L-E-C-V-H-R | (SEQ ID NO:310) |
| K-Y-W-C-T-Q-W-G-L-K-C-D-K-Q | (SEQ ID NO:311) |
| K-Y-W-C-S-F-W-G-L-Q-C-K-T | (SEQ ID NO:312) |
| R-Y-W-C-N-F-W-G-V-N-C-D-A-N | (SEQ ID NO:313) |
| N-Y-W-C-T-H-W-G-V-M-C-L-D-H | (SEQ ID NO:314) |
| Y-W-F-C-K-W-F-P-S-Q-C-Q-F-M | (SEQ ID NO:315) |
| A-Y-W-C-K-Q-W-G-L-K-C-Q-L-G | (SEQ ID NO:316) |
| K-Y-W-C-K-F-W-G-L-E-C-K-V-G | (SEQ ID NO:317) |
| N-Y-W-C-T-E-W-G-L-N-C-N-N-K | (SEQ ID NO:318) |
| S-Y-W-C-E-K-W-G-L-T-C-E-T-H | (SEQ ID NO:319) |
| E-Y-W-C-R-I-W-G-L-Q-C-N-M-V | (SEQ ID NO:320) |
| K-Y-W-C-K-K-W-G-V-N-C-D-F-N | (SEQ ID NO:321) |
| K-Y-W-C-S-V-W-G-V-Q-C-P-H-S | (SEQ ID NO:322) |
| F-Y-W-C-T-K-W-G-L-E-C-I-H-S | (SEQ ID NO:323) |
| H-Y-W-C-Q-Q-W-G-L-M-C-F-E-T | (SEQ ID NO:324) |
| K-Y-W-C-K-R-W-G-L-M-C-N-G-G | (SEQ ID NO:325) |
| A-Y-W-C-M-T-W-G-V-P-C-I-S-W | (SEQ ID NO:326) |
| K-Y-W-C-K-K-W-G-V-N-C-D-F-N | (SEQ ID NO:327) |
| K-Y-W-C-S-V-W-G-V-Q-C-P-D-S | (SEQ ID NO:328) |
| K-Y-W-C-S-V-W-G-V-Q-C-P-H-S | (SEQ ID NO:329) |
| L-Y-W-C-T-K-W-G-V-T-C-Q-K-D | (SEQ ID NO:330) |
| T-Y-W-C-H-K-W-G-V-K-C-A-T-T | (SEQ ID NO:331) |
| T-Y-W-C-T-F-W-E-L-P-C-D-P-A | (SEQ ID NO:332) |
| K-Y-W-C-T-K-W-Q-L-N-C-E-E-V | (SEQ ID NO:333) |
| N-Y-W-C-H-F-W-Q-V-P-C-L-E-Q | (SEQ ID NO:334) |
| T-Y-W-C-V-V-W-N-V-P-C-S-T-D | (SEQ ID NO:335) |
| N-F-W-C-H-T-W-G-L-Q-C-N-D-L | (SEQ ID NO:336) |
| F-W-Y-C-Y-W-F-N-E-K-C-K-T-P | (SEQ ID NO:337) |
| G-F-W-C-T-F-W-G-V-T-C-E-A-G | (SEQ ID NO:338) |
| P-H-N-C-D-D-H-Y-W-Y-C-K-W-F | (SEQ ID NO:339) |
| E-M-T-C-S-S-H-Y-W-Y-C-T-W-M | (SEQ ID NO:340) |
| H-I-D-C-K-T-N-Y-W-W-C-R-W-T | (SEQ ID NO:341) |
| E-M-R-C-G-Q-H-F-W-Y-C-E-W-F | (SEQ ID NO:342) |
| N-Y-W-C-N-F-W-Q-L-P-T-C-D-N-L | (SEQ ID NO:343) |
| Y-W-Y-C-Q-W-F-Q-E-V-N-K-C-F-N-S | (SEQ ID NO:344) |
| Y-Y-W-C-R-H-W-F-P-D-F-D-C-V-H-S | (SEQ ID NO:345) |
| Y-W-Y-C-S-W-F-P-D-R-P-D-C-P-L-Y | (SEQ ID NO:346) |
| Y-W-Y-C-V-W-F-D-N-A-D-Q-C-V-H-H | (SEQ ID NO:347) |
| A-A-T-C-S-T-S-Y-W-Y-Y-Q-W-F-C-T-D-S | (SEQ ID NO:348) |
| Y-W-A-C-V-W-G-L-K-S-C-V-D-R | (SEQ ID NO:349) |
| Y-W-R-C-V-W-F-P-A-S-C-P-T | (SEQ ID NO:350) |
| D-W-Q-C-L-W-W-G-N-S-F-W-P-Y-C-A-N-L | (SEQ ID NO:351) |
| F-W-R-C-H-W-W-P-E-R-C-P-V-D | (SEQ ID NO:352) |
| N-P-M-C-W-K-K-S-W-W-E-D-A-Y-C-I-N-H | (SEQ ID NO:353) |
| S-W-V-C-W-K-A-K-W-W-E-D-K-R-C-A-P-F | (SEQ ID NO:354) |
| S-R-Q-C-W-K-E-L-W-W-T-D-Q-M-C-L-D-L | (SEQ ID NO:355) |
| S-F-R-C-Q-S-S-F-P-S-W-Y-C-D-Y-Y | (SEQ ID NO: 356) |
| S-W-H-C-Q-N-T-Y-P-E-W-Y-C-Q-W-Y | (SEQ ID NO:357) |
| G-S-K-C-K-Q-T-G-F-P-R-W-W-C-E-H-Y | (SEQ ID NO:358) |
| D-G-V-C-G-P-R-G-F-G-P-A-W-F-C-M-H-Y | (SEQ ID NO:359) |
| Y-S-H-C-A-T-H-Y-P-T-W-Y-C-L-H-F | (SEQ ID NO:360) |
| F-C-N-C-W-G-S-H-E-F-T-F-C-V-D-D | (SEQ ID NO:361) |
| P-G-W-C-Y-S-D-I-W-G-F-K-H-F-C-N-L-D | (SEQ ID NO:362) |
| D-S-S-C-I-K-H-H-N-K-V-T-C-F-F-P | (SEQ ID NO:363) |
| R-W-S-C-W-G-V-W-G-C-V-W-V | (SEQ ID NO:364) |
| P-V-D-C-K-H-H-F-W-W-C-Y-W-N | (SEQ ID NO:365) |
| S-W-N-C-A-F-H-H-N-E-M-V-W-C-D-D-G | (SEQ ID NO:366) |
| Y-W-Y-C-W-F-P-D-R-P-E-C-P-L-Y | (SEQ ID NO:367) |
| N-P-M-C-W-R-A-S-W-W-E-D-A-Y-C-I-N-H | (SEQ ID NO:409) |
| N-P-M-C-W-R-A-H-W-W-E-D-A-Y-C-I-N-H | (SEQ ID NO:410) |
| E-H-M-C-V-Y-T-T-W-G-E-L-I-W-C-D-N-H | (SEQ ID NO:411) |
| A-C-V-Y-T-T-W-G-E-L-I-W-C-D-N-H | (SEQ ID NO:412) |
| T-C-V-Y-T-T-W-G-E-L-I-W-C-D-N-H | (SEQ ID NO:413) |
| E-C-V-Y-T-T-W-G-E-L-I-W-C-D-N-H | (SEQ ID NO:414) |
| V-C-V-Y-T-T-W-G-E-L-I-W-C-D-N-H | (SEQ ID NO:415) |
| [Nle]-C-V-Y-T-T-W-G-E-L-I-W-C-D-N-H | (SEQ ID NO:416) |
| S-R-A-C-S-R-D-W-S-G-A-L-V-W-C-A-G-H | (SEQ ID NO:417) |
| E-R-A-C-S-R-D-W-S-G-A-L-V-W-C-A-G-H | (SEQ ID NO:418) |
| A-C-S-R-D-W-S-G-A-L-V-W-C-A-G-H | (SEQ ID NO:419) |
| T-C-S-R-D-W-S-G-A-L-V-W-C-A-G-H | (SEQ ID NO:420) |
| E-C-S-R-D-W-S-G-A-L-V-W-C-A-G-H | (SEQ ID NO:421) |
| V-C-S-R-D-W-S-G-A-L-V-W-C-A-G-H and | (SEQ ID NO:422) |
| G-C-S-R-D-W-S-G-A-L-V-W-C-A-G-H. | (SEQ ID NO:423) |

N-terminal and/or C-terminal truncations of the above Fc-region binding polypeptides can also be used, particularly cyclic polypeptides that retain binding affinity for antibody Fc-regions.

Fc-region binding molecules according to the above formulae can include the following: polypeptides of formula I, in which X1 is G; X2 is A or E; X3 is L; and X4 is I or V; polypeptides of formula II, in which X is F or Y; and in the tripeptide of formula IIA, X1 is C or Y; X2 is C, K, N or T; and X3 is F, I, K, Q or V.

Particular examples of immunoglobulin binding molecules include proteins that include the following polypeptides:

| | |
|---|---|
| RRACSRDWSGALVWCAGH; | (SEQ ID NO:238) |
| DHMCVYTTWGELIWCDNH; | (SEQ ID NO:260) |
| KYWCSFWGLQCKT; | (SEQ ID NO:312) |
| PVDCKHHFWWCYWN; | (SEQ ID NO:365) |
| DDHCYWFREWFNSECPHG; | (SEQ ID NO:274) |
| YYWCNYWGLCPDQ; | (SEQ ID NO:280) |
| PHNCDDHYWYCKWF; | (SEQ ID NO:339) |
| SYWCKIWDVCPQS; | (SEQ ID NO:281) |
| KYWCNLWGVCPAN; | (SEQ ID NO:282) |
| AATCSTSYWYYQWFCTDS; | (SEQ ID NO:348) |
| TYWCTFWELPCDPA; | (SEQ ID NO:332) |
| YWYCWFPDRPECPLY; | (SEQ ID NO:367) |
| SWVCWKAKWWEDKRCAPF; | (SEQ ID NO:354) |
| NPMCWKKSWWEDAYCINH; and | (SEQ ID NO:353) |
| SWNCAFHHNEMVWCDDG. | (SEQ ID NO:366) |

Still other exemplary polypeptides can have the following sequences, and may include optional amino-terminal (e.g., acetylation) and carboxy-tenninal modifications (e.g., amidation):

| | |
|---|---|
| GDDHMCVYTTWGELIWCDNHEPGPEGGGK; | (SEQ ID NO:368, designated DX249) |
| AGKYWCSFWGLQCKTGTPGPEGGGK; | (SEQ ID NO:370, designated DX250) |
| AGPVDCKHHFWWCYWNGTPGPEGGGK; | (SEQ ID NO:377, designated DX251) |
| GDDDHCYWFREWFNSECPHGEPGPEGGGK; | (SEQ ID NO:378, designated DX252) |
| GDRRACSRDWSGALVWCAGHEPGPEGGGK; | (SEQ ID NO:369, designated DX253) |
| AGYYWCNYWGLCPDQGTPGPEGGGK; | (SEQ ID NO:379, designated DX254) |
| AGPHNCDDHYWYCKWFPGPEGGGK; | (SEQ ID NO374, designated DX389) |
| AGSYWCKIWDVCPQSPGPEGGGK; | (SEQ ID NO:371, designated DX392) |
| AGKYWCNLWGVCPANPGPEGGGK; | (SEQ ID NO:372, designated DX395) |
| AGAATCSTSYWYYQWFCTDSPGPEGGGK; | (SEQ ID NO:375, designated DX398) |
| AGTYWCTFWELPCDPAPGPEGGGK; | (SEQ ID NO:373, designated DX404) |
| AGYWYCWFPDRPECPLYPGPEGGGK; | (SEQ ID NO:376, designated DX413) |
| GDSWVCWKAKWWEDKRCAPFGTPGPEGGGK; | (SEQ ID NO:380, designated DX595) |
| GDNPMCWKKSWWEDAYCINHGTPGPEGGGK; | (SEQ ID NO:381, designated DX596) |
| GDSWNCAFHHNEMVWCDDGGTPGPEGGGK; | (SEQ ID NO:382, designated DX597) |
| GDWGECTVTSYGELIWCGGLEPGPEGGGK; | (SEQ ID NO:383, designated DX1070) |
| GDNPMCWRASWWEDAYCINHEPGPEGGGK; | (SEQ ID NO:384, designated DX1071) |
| GDNPMCWRAHWWEDAYCINHEPGpPGGGK; | (SEQ ID NO:385, designated DX1072) |
| GDDHMCVYTTWGELIWCDNHEPGPEG-J-NH2 (SEQ ID NO:386, designated DX877) | |
| GDDHMCVYTTWGELIWCDNHEPG-J-Su-J-NH2 (SEQ ID NO:387, designated DX878) | |
| GDDHMCVYTTWGELIWCDNHEPG-J-Z-J-NH2 (SEQ ID NO:388, designated DX905) | |
| GDDHMCVYTTWGELIWCDNH-J-NH2 (SEQ ID NO:389, designated DX907) | |
| GDDHMCVYTTWGELIWCDNH-J-Su-J-NH2 (SEQ ID NO:390, designated DX909) | |
| GDDHMCVYTTWGELIWCDNH-J-Z-J-NH2 (SEQ ID NO:391, designated DX911) | |
| DHMCVYTTWGELIWCDNHEPEGGGK; | (SEQ ID NO:392, designated DX1062) |
| EHMCVYTTWGELIWCDNHEPGPEGGGK; | (SEQ ID NO:393, designated DX1063) |
| ACVYTTWGELIWCDNHEPGPEGGGK; | (SEQ ID NO:394, designated DX1064) |
| TCVYTTWGELIWCDNHEPGPEGGGK; | (SEQ ID NO:395, designated DX1065) |
| ECVYTTWGELIWCDNHEPGPEGGGK; | (SEQ ID NO:396, designated DX1066) |
| VCVYTTWGELIWCDNHEPGPEGGGK; | (SEQ ID NO:397, designated DX1067) |
| Ac-[Nle]CVYTTWGELIWCDNHEPGPEGGGK; | (SEQ ID NO:398, designated DX1068) |
| CVYTTWGELIWCDNHEPGPEGGGK; | (SEQ ID NO:399, designated DX1069) |
| SRACSRDWSGALVWCAGHEPGPEGGGK; | (SEQ ID NO:400, designated DX1139) |
| RRACSRDWSGALVWCAGHEPGPEGGGK; | (SEQ ID NO:401, designated DX1142) |
| ERACSRDWSGALVWCAGHEPGPEGGGK; | (SEQ ID NO:402, designated DX1141) |
| ACSRDWSGALVWCAGHEPGPEGGGK; | (SEQ ID NO:403, designated DX1142) |
| TCSRDWSGALVWCAGHEPGPEGGGK; | (SEQ ID NO:404, designated DX1143) |
| ECSRDWSGALVWCAGHEPGPEGGGK; | (SEQ ID NO:405, designated DX1144) |
| VCSRDWSGALVWCAGHEPGPEGGGK; | (SEQ ID NO:406, designated DX1145) |
| GCSRDWSGALVWCAGHEPGPEGGGK; and | (SEQ ID NO:407, designated DX1146) |
| CSRDWSGALVWCAGHEPGPEGGGK-NH2. | (SEQ ID NO:408, designated DX1147) |

With respect to the foregoing polypeptides, the polypeptides can further include a chemical modification, e.g., N-terminal acetylation and/or C-terminal amidation: e.g., one of the following: -J-NH₂ denotes the C-terminal group —NH—(CH₂CH₂O)₂—CH₂CH₂—NH₂, -J-Su-J-NH₂ denotes the C-terminal group —NH—(CH₂CH₂O)₂—CH₂CH₂—NH—C:O—CH₂CH₂—C:O—NH(CH₂CH₂O)₂—CH₂CH₂—NH₂, -J-Z-J-NH₂ denotes the C-terminal group —NH—(CH₂CH₂O)₂—CH₂CH₂—NH—C:O—CH₂—O—(CH₂CH₂O)₂—CH₂—C:O NH—(CH₂CH₂O)₂—CH₂CH₂—NH₂, and [Nle] denotes norleucine.

The immunoglobulin binding polypeptides can have high affinity (e.g., K_{D} in the range 10 μM to 0.01 μM, more preferably in the range 1.0 μM to 0.01 μM) for human Fc polypeptides or particular IgG isotypes (e.g., IgGI, IgG2, IgG3 and/or IgG4). Some polypeptides also show species specificity (e.g., binding to human but not other mammalian IgGs). For example:

DX249 exhibits dissociation constants (K_{D}) for human IgGI of less than 0.1 μM at pH 5.7 and less than 0.5 μM at pH 7.4;

DX252 exhibits dissociation constants (K_{D}) for human IgG3 of less than 0.1 μM at pH 5.7 and in the range of 2.1 μM to 3.4 μM for IgGI, IgG2, IgG3, and IgG4 at pH 7.4;

DX253 exhibits quantitative binding of Fc protein (capture efficiency >90% of total load) from buffer solution and tobacco extract;

DX254 exhibits dissociation constants (K_{D}) for human IgG1 of less than 0.1 μM at pH 5.7, less than 2.0 μM at pH 7.4, and less than 1.0 μM at pH 9.3;

DX301 exhibits dissociation constants below about 10 μM for human Fe, IgG1, IgG2 and IgG4; and

DX300 exhibits a dissociation constant of 4.1±4.6 for human IgG3. Variants of the above peptides can also be used, including the segment DX249-A, DHMCVYTTWGELIWCDNH (SEQ ID NO: 260); the segment DX253-A, RRACSRDWSGALVWCAGH (SEQ ID NO: 238); and AATCSTSYWYYQWFCTDS (SEQ ID NO: 348).

The term “associated” refers to a direct or indirect physical attachment between compounds. Attachments can be mediated by a covalent or non-covalent interaction. An indirectly physical attachment refers to, for example, a case where two compounds are not in direct contact with each other, but each contact one or more intermediary compounds.

The term “polypeptide” refers to a polymer of three or more amino acids linked by a peptide bond. The polypeptide may include one or more unnatural amino acids. Typically, the polypeptide includes only natural amino acids. The term “peptide” refers to a polypeptide that is between three and thirty-two amino acids in length. A protein can include one or more polypeptide chains.

The term “antibody” as used herein refers to an immunoglobulin molecule or immunologically active portion thereof, i.e., an antigen-binding portion. An antibody can include at least one, and preferably two, heavy (H) chain variable regions (abbreviated herein as VH), and at least one and preferably two light (L) chain variable regions (abbreviated herein as VL). The VH and VL regions can be further subdivided into regions of hypervariability, termed “complementarity determining regions” (“CDR”), interspersed with regions that are more conserved, termed “framework regions” (FR). The extent of the framework region and CDR's has been precisely defined (see, Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917). Each VH and VL is composed of three CDR's and four FRs, arranged from amino-terminus to carboxy-terminus in the following order: FR1, CDR1, FR2, CDR2, FR3, CDR3, FR4.

As used herein, the term “immunoglobulin” refers to a protein consisting of one or more polypeptides substantially encoded by immunoglobulin genes. Some human immunoglobulin genes include the kappa, lambda, alpha (IgA1 and IgA2), gamma (IgG1, IgG2, IgG3, IgG4), delta, epsilon and mu constant region genes, as well as the myriad immunoglobulin variable region genes. Full-length immunoglobulin “light chains” (about 25 KDa or 214 amino acids) are encoded by a variable region gene at the NH₂-terminus (about 110 amino acids) and a kappa or lambda constant region gene at the COOH—terminus. Full-length immunoglobulin “heavy chains” (about 50 KDa or 446 amino acids), are similarly encoded by a variable region gene (about 116 amino acids) and one of the other aforementioned constant region genes, e.g., gamma (encoding about 330 amino acids).

The term “antigen-binding fragment” of an antibody (or simply “antibody portion,” or “fragment”), as used herein, refers to one or more fragments of a full-length antibody that retain the ability to specifically bind to the antigen. Examples of antigen-binding fragments include, but are not limited to: (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al (1988) Proc. Natl. Acad. Sci. USA 85:5879-5883). Such single chain antibodies are also encompassed within the term “antigen-binding fragment” of an antibody. These antibody fragments are obtained using conventional techniques (including immunization, phage display, and CDR grafting) known to those with skill in the art, and the fragments are screened for utility in the same manner as are intact antibodies.

An “isolated composition” refers to a composition that is removed from at least 90% of at least one component of a natural sample from which the isolated composition can be obtained.

The invention includes sequences and variants that include one or more substitutions, e.g., between one and six substitutions. Whether or not a particular substitution will be tolerated, i.e., will not adversely affect desired biological properties, such as binding activity can be determined as described in Bowie, et al. (1990) Science 247:1306-1310. One or more or all substitutions may be conservative. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Still other substitutions may insert a non-naturally occurring amino acid.

All patent applications, patents, and references cited herein are incorporated by reference in their entirety. Accordingly, U.S. provisional applications Ser. No. 60/331,352 filed Mar. 9, 2001, Ser. No. 60/292,975 filed May 23, 2001, Ser. No. 60/284,534, filed Apr.18, 2001, Ser. No. 10/094,401, filed Mar. 8, 2002, and Ser. No. 10/125,869, filed Apr. 18, 2002, U.S. Published application 2003/0069395 are incorporated by reference for all purposes in their entirety.

### BRIEF DESCRIPTION OF THE DRAWINGS

FIG. 1 is an image of a two-dimensional gel of a proteins in a fraction of affinity-purified serum albumin and associated proteins.

### DETAILED DESCRIPTION

Serum proteins are important components of the circulatory system and have wide spread function in physiology and the immune response, among other roles. Characterization of compounds associated with serum proteins provide useful indicia for studying, diagnosing, and monitoring a subject. Serum proteins can be isolated using an affinity reagent. Compounds covalently or non-covalently associated the isolated serum proteins are analyzed, e.g., to determine their identity and/or abundance.

Isolating Serum Albumin and Associated Compounds

In one implementation, human serum albumin (HSA) is isolated from a sample, e.g., blood, plasma, or serum. Compounds associated with the serum albumin are then analyzed.

1. In a first example, a serum sample is applied to an insoluble matrix that includes one or more affinity ligands for HSA. Examples of affinity ligands include peptide ligands described below. The matrix is washed with a physiological-strength buffer (e.g., phosphate buffered saline), or more stringent buffers (e.g., including higher ionic strengths, detergents, chaotropes, and the like).

HSA and any compounds associated with it are eluted from the matrix, and recovered. Elution can be achieved, for example, by applying an appropriate buffer that favors the dissociation of HSA from the affinity ligand or by separating the affinity ligand from the insoluble matrix.

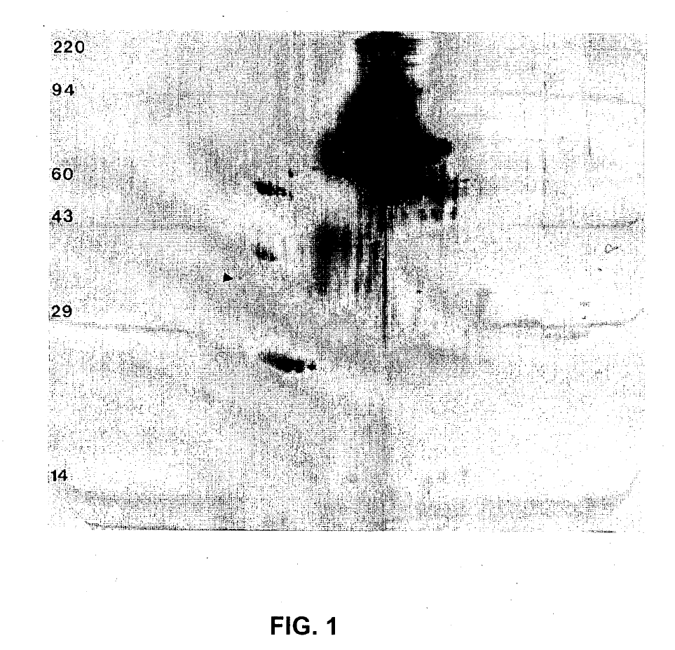

In the eluted material, serum albumin may represent a substantial fraction of the purified composition (see, e.g., FIG. 1 and Example 1). Hence, the associated compounds may constitute less than 10% of the sample. At least some of the associated compounds may be proteinaceous, e.g., peptides, polypeptides, or protein complexes. For example, in the case of protein complexes, at least some of the associated compounds may be indirectly associated with HSA. Other compounds may be metabolites, small molecules (e.g., having a molecular weight of less than 5000 or 1000 Daltons),

2. In a second example, a sample is applied to an insoluble matrix that includes one or more affinity ligands for HSA. The matrix is washed with a physiological-strength buffer (e.g., phosphate buffered saline), but not more stringent buffers.

HSA and any compounds associated with it are removed from the matrix by separating the affinity ligands from the matrix (e.g., the affinity ligands can be attached to the matrix by a covalent bond that is cleaved).

This preparation is then applied to an insoluble matrix that includes a thiol-reactive group, e.g., an activated maleimide or iodoacetamide (see also “Thiol Reactive Compounds,” below). The activated maleimide, for example, reacts with the free cysteine of HSA, cysteine 34. Few other abundant proteins include a free cysteine when isolated from serum or from an oxidized sample. After reaction, the matrix is washed to remove proteins and other compounds that are not associated with HSA.

Compounds associated with HSA can be released from the matrix by one or more of following processes:

(a) HSA can be denatured with a chaotrope or other denaturing conditions. The denatured HSA remains covalently bound to the matrix, while non-covalently associated compounds are released from the matrix. Denaturants can be applied to the matrix incrementally, e.g., in a step or continuous gradient.

Examples of chaotropes include guanidinium HCI (e.g., >4, 5, or 6M) or urea (e.g., >6 or 8M). Examples of other denaturing conditions include acid (e.g., phosphoric acid, pH 1), ionic detergents (e.g., 1% SDS, or greater), heat (e.g., >60° C.) boiling or an organic solvent (e.g., acetonitrile).

(b) Associated compounds can be eluted by competition using an affinity ligand that binds to HSA (e.g., an antibody, a peptide ligand, or a compound known to bind HSA, e.g., a long chain fatty acid, a drug, e.g., a drug listed in Table 1, or an endogenous compound, e.g., an endogenous compound listed in Table 2). This process may specifically elute compounds that associate with a particular epitope of HSA.

(c) Associated compounds can be separated from each other by selective elution, e.g., using a step or gradient elution, in which a solution parameter is altered (e.g., ionic strength, pH, chaotrope concentration). Fractions can be collected, and individually analyzed. This process, for example, can used to obtain preparations that include a subset of the associated compounds.

Fractions of eluted associated compounds can be subjected to additional purification steps, e.g., a preparative or analytic process described in Scopes (1994) Protein Purification: Principles and Practice, New York: Springer-Verlag.

The methods described herein can also be used to isolate serum albumins from other species, e.g., a non-human mammalian species and non-mammalian species.

Peptide Ligands that Bind Serum Albumin.

In some embodiments, peptide ligands are used to isolate a serum albumin and associated proteins. Provisional patent applications Ser. No. 60/331,352 filed Mar. 9, 2001 and Ser. No. 60/292,975 filed May 23, 2001 describe a number of exemplary peptide ligands that bind to serum albumin. Some exemplary peptide ligands include DX-321, DX-321-A, DX-321-B, DX-236, DX-236-A, and DX-236B.

DX-321 includes the peptide sequence:

AEGTGDRNMCKFSWIRSPAFCARADPE(SEQ ID NO: 40). DX-321 binds to human serum albumin and is useful for isolating human serum albumin and associated compounds.

DX-321-A includes the peptide sequence:

RNMCKFSWIRSPAFCARA (SEQ ID NO: 215).

DX-321-B includes the peptide sequence:

CKFSWIRSPAFC (SEQ ID NO: 120)

DX-236 includes the peptide sequence:

AEGTGDFWFCDRIAWYPQHLCEFLDPEGGGK(SEQ ID NO: 19). DX-236 binds at least to a number of mammalian serum albumins and is useful under appropriate conditions as a serum albumin-binding agent that binds to serum albumins from multiple species.

DX-236A includes the peptide sequence:

FWFCDRIAWYPQHLCEFLD (SEQ ID NO: 210)

DX-236B includes the peptide sequence:

CDRIAWYPQHLC (SEQ ID NO: 9)

In one implementation, an affinity matrix for purifying serum albumin includes a plurality of binding ligands, e.g., binding ligands having specificity for different epitopes on the serum albumin. For example, an affinity matrix for binding HSA can include two different species of HSA binding peptides, e.g., DX-236 and DX-321.

In addition to peptide ligands, larger polypeptide ligands can be used, e.g., protein that include at least one immunoglobulin domain, e.g., an antibody or antibody fragment.

Exemplary Serum Albumin Associated Compounds

A number of endogenous and exogenous compounds are known to associated with serum albumin. A method described herein can include determining whether one or more of such compounds (e.g., a compound in Table 1 or Table 2) is associated with an isolated serum albumin. Compounds other than serum albumin can also be evaluated.

TABLE 1
Drugs that bind to Serum Albumin

| | |
|---|---|
| Salicylate | Sulfisoxazole |
| Warfarin | Phenylbutazone |
| Digitoxin | Indomethacin |
| Tolbutamide | Furosenmide |
| Phenytoin | Chlorpropamide |
| Chlorthiazide | Oxacillin |
| Benzylpenicilliln | Acetotrizoate |
| Phenol Red | Bromscesol green |
| Bromophenol Blue | Iophenoxate |
| Sulfobromophthalein | Methyl organge |
| Methyl Red | Evans blue |
| Diazepam (S) | Ibuprofen |
| Naproxen | Octanoate |
| Chlofibrate | Chlorpromazine |
| Imipramine | Quinidine |

TABLE 2
Endogenous compounds that bind Serum Albumin

| | |
|---|---|
| Long-chain fatty acids | Eicosanoids |
| Bile acids | |
| Steroids | Cortisol |
| Progesterone | Testosterone |
| Aldosterone | |
| Hematin | Bilirubin |
| L-Thyroxine | L-Tryptophan |
| 25-OH-Vitamin D₃ | 1,25-(OH)₂-Vitamin D₃ |
| Aquocobalamin | Folate |
| Ascorbate | |
| Copper(II) | Zinc(II) |
| Calcium | Magnesium |
| Chloride | |

Thiol Reactive Compounds

As described above, thiol reactive groups can be used to immobilize a serum protein that includes a free cysteine. In particular, serum albumin is an abundant serum protein that includes a free cysteine. For example, cysteine 34 of HSA is typically available for coupling. Exemplary thiol reactive groups include the following.

Halogen derivatives. Haloacetyl compounds and benzyl halides, particularly iodo and bromo derivatives, can be reacted with cysteines. For example, iodoacetate can be used to react with cysteines. The reaction is more specific if the iodoacetate is present in limiting quantities related to the number of available sulfhydryl and under alkaline pH.

Maleimides. The double bond of maleimides (maleic acid imides) can undergo an alkylation reaction with sulfhydryl groups, resulting in a thioether bond. Maleimides are particularly specific for sulfhydryls between pH 6.5 and 7.5.

Thiol-Disulfide Exchange Reagents. Cysteines can also be crosslinked using compounds that have a disulfide bond and undergo disulfide exchange with the free cysteine on the serum protein. Pyridyl disulfides, for example, can be generated by reaction of 2-iminothiolane with 4,4′ dipyridyl disulfide.

Still other thiol reactive compounds include aziridines, acryloyl derivaties, and arylating reagents (such as 2,4 dinitrofluorobenzye). See also Hermanson (1996) “Section 2: Thiol-Reactive Chemical Reactions” of Bioconjugate Techniques Academic Press.

Peptide Ligands that Bind Immunoglobulins

In another implementation, a soluble immunoglobulin is isolated from a sample, e.g., blood, plasma, or serum. Compounds associated with the immunoglobulin are then analyzed.

A peptide ligand can be used to isolate the soluble immunoglobulin. WO 2002/086070 and provisional patent application 60/284,534, filed Apr. 18, 2001, describe a number of exemplary peptide ligands that bind to the Fc region of an immunoglobulin. For example:

DX249, GDDHMCVYTTWGELIWCDNHEPGPEGGGK (SEQ ID NO: 368) which exhibits dissociation constants (K_{D}) for human IgG1 of less than 0.1 μM at pH 5.7 and less than 0.5 μM at pH 7.4, the segment DX249-A, DHMCVYTTWGELIWCDNH (SEQ ID NO: 260), or the segment DX-249-B, CVYTTWGELIWC (SEQ ID NO: 427);

DX253, GDRRACSRDWSGALVWCAGHEPGPEGGGK (SEQ ID NO: 369), exhibits quantitative binding of Fc protein (capture efficiency >90% of total load), the segment DX253-A, RRACSRDWSGALVWCAGH (SEQ ID NO: 238), or the segment DX253-B, CSRDWSGALVWC (SEQ ID NO: 428);

DX398, AGAATCSTSYWYYQWFCTDSPGPEGGGK (SEQ ID NO: 375), DX398-A: AATCSTSYWYYQWFCTDS (SEQ ID NO: 348) or DX398-B: CSTSYWYYQWFC (SEQ ID NO: 429);

DX252, GDDDHCYWFREWFNSECPHGEPGPEGGGK (SEQ ID NO: 378), exhibits dissociation constants (K_{D}) for human IgG3 of less than 0.1 μM at pH 5.7 and in the range of ˜2.1 μM to ˜3.4 μM for IgG1, IgG2, IgG3, and IgG4 at pH 7.4; and

DX254, AGYYWCNYWGLCPDQGTPGPEGGGK (SEQ ID NO: 379), exhibits dissociation constants (K_{D}) for human IgG1 of less than 0.1 μM at pH 5.7, less than 2.0 μM at pH 7.4, and less than 1.0 μM at pH 9.3.

In one implementation, a sample is contacted to an affinity matrix that includes ligands that bind to immunoglobulins. Immunoglobulin and associated compounds are isolated. In one example, the isolated material is analyzed, e.g., to characterize antigens associated with immunoglobulin. In another example, the isolated material is cultured, e.g., to identify a pathogen bound by the immunoglobulin.

Analyzing Associated Compounds

A fraction of a serum protein and associated compounds can be analyzed by a number of processes. Exemplary methods for analyzing proteinaceous compounds associated with a serum protein include: sodium dodecyl sulfate polyacrylamide gel electrophoresis (SDS-PAGE), 2-D gel electrophoresis (iso-electric focusing and PAGE), HPLC, FPLC (ion-chromatography, size exclusion chromatography, hydrophobic interaction chromatography, and the like), immuno-analysis (e.g., immuno-blots, enzyme-linke.d immunosorbent assay (ELISA), immunoprecipitation, and the like), mass spectroscopy, and protein sequencing.

Exemplary methods for analyzing a non-proteinaceous compound associated with a serum protein include: mass spectroscopy (e.g., GC-mass spec or “GC/MS”), thin-layer chromatography, and other chemical detection methods.

1D gels. SDS-PAGE can be used to separate proteins by their apparent/approximate molecular weight in an acrylamide gel. The concentration of acrylamide can be varied according to the expected size or a gradient of acrylamide concentration can be used. After electrophoresis, proteins can be stained using a variety of dyes, include Coomassie Blue, silver stains, and fluorescent dyes such as Sypro Red (Molecular Probes, Inc., Eugene, Oreg.). The acrylamide gel can be imaged, e.g., to determine relative concentration of resolved bands of proteins. The image can be stored in a computer database.

2D gels. This method can be used to separate polypeptides according to two properties, isoelectric point (pI) and apparent molecular weight (MW). Proteins are first separated by PI (isoelectric focusing) and then separated according to apparent molecular weight by SDS-PAGE. The 2D gel is stained and imaged. The detected “spots” of proteins provide information about the identity, modification state, and relative abundance of each protein. Proteins can also be excised from spots and further characterized by mass spectroscopy or protein sequencing.

Isoelectric focusing for the first dimension can utilize immobilized pH gradient strips, e.g., in which polycarboxylic acid ampholytes are immobilized. Strips can be produced that focus within a desired pH range, both narrow and wide. A strip can be selected for the appropriate degree of resolution.

After isoelectric focusing, protein in the strip are denatured and reduced, e.g., using SDS and a thiol reductant. The strip is attached to an SDS-PAGE slab gel and proteins are separated by molecular weight.

Immuno-Detection. Antibodies, either characterized or uncharacterized, can be used to identify compounds associated with a serum protein. The antibodies can be applied to a separated sample (e.g., an electrophoresed sample, as in a Western blot) or to the sample as a whole (e.g., an ELISA). An antibody can also be used to immunoprecipitate the compound. The antibody can be coupled to a label or signal generator to enable detection of the compound. Antibody fragments and other derivatives can also be used.

Mass Spectroscopy. Time-of flight mass spectrometry (TOF-MS) and electrospray mass spectrometry can be used to characterize compounds associated with a serum protein. TOF-MS is sensitive, highly accurate, and rapid (R. J. Cotter, (1992) Anal. Chem. 64:1027. For example, TOF-MS can record a complete mass spectrum on a microsecond timescale.

One common TOF-MS method is matrix-assisted laser desorption/ionization (MALDI) (Karas and Hillenkamp, Anal. Chem. 60, 2299 (1988). This method is amenable to the mass spectrometry to oligonucleotides and nucleic acids. See generally, P. Limbach et al, “Characterization of oligonucleotides and nucleic acids by mass spectrometry”, In Current Opinion in Biotechnology, 6, 96-102 (1995).

Mass spectroscopy can be combined with protease digestion to determine the precise molecular weight of proteolytic fragments of a protein. This information can be compared to a computer sequence database to infer the sequence of the protein. For example, the database can includes predicted protein sequences from genome sequence. See, e.g., Zhang and Chait (2000) Anal. Chem. 72:2482. Mass spectroscopy can also be used to determine the modification state of a protein (e.g., oxidation, glycosylation).

Exemplary proteases for mapping proteolytic fragments include: elastase, trypsin, chymotrypsin, pepsin, papain, and Glu-C. Certain chemical agents can also be used, e.g., formic acid and cyanogen bromide.

For matrix-assisted laser desorption/ionization mass spectrometry (MALDI-MS or MALDI-TOF), a proteolyzed sample is combined with a matrix (e.g., α-cyano-4-hydroxycinnamic acid), sinapinic acid, or gentisic acid), and dried on a mass spectroscopy plate. The plate is then placed in a mass spectrometer where the protein fragments are ionized, and then analyzed for their time of flight. Accurate molecular weights are determined from these measurements.

Protein Sequencing. The N-termini of a purified protein (e.g., 2 to 5 picomoles of the protein) can be sequenced by Edman degradation. This process can be automated. N-terminal sequence information can be combined with mass spectroscopy information and comprehensive databases to unambiguously identify a protein.

Peptide and Protein Arrays

In some implementations, an array of proteins and/or peptide ligands, at least some of which bind to serum proteins can be used. For example, the array can include one or more peptide ligands described herein. In a particular example, the array includes at least two different peptide ligands that bind to serum albumin.

In general, a sample is contacted to the array, and complexes within the sample are allowed to bind to ligands on the array, e.g., so that different complexes of a serum binding protein are isolated by the different ligands. Each discrete address can be evaluated separately.

Methods of producing polypeptide arrays are described, e.g., in De Wildt et al. (2000) Nat. Biotechnol. 18:989-994; Lueking et al. (1 999) Anal. Biochem. 270:103- 111; Ge (2000) Nucleic Acids Res. 28, e3, I-VII; MacBeath and Schreiber (2000) Science 289:1760-1763; U.S. Pat. No. 2002-192,673 WO 01/98534, WO01/83827, WO02/12893, WO 00/63701, WO 01/40803 and WO 99/51773. In some implementations, polypeptides (including peptides) are spotted onto discrete addresses of the array, e.g., at high speed, e.g., using commercially available robotic apparati, e.g., from Genetic MicroSystems or BioRobotics. The array substrate can be, for example, nitrocellulose, plastic, glass, e.g., surface-modified glass. The array can also include a porous matrix, e.g., acrylamide, agarose, or another polymer.

Arrays of peptides can be similarly produced. In addition, peptides can be directly synthesized in an array format, e.g., according to U.S. Pat. No. 5,143,854. It may also be possible to use nucleic acid aptamers as ligands to isolate serum proteins and associated complexes.

Analysis of Altered or Abnormal States

The methods described herein can be used to characterize an altered or abnormal state of a subject. For example, a profile of compounds associated with one or more serum proteins can be determined for the subject at one or more instances. The subject can be a diseased subject, a genetically altered subject, a subject afflicted by a genetic disorder, a subject exposed to toxins (e.g., environmental toxins, narcotics, and so forth), or a subject receiving a treatment. For instance, in the case of monitoring a treatment of a subject, the profile can be determined prior to treatment, and at regular intervals after treatment. The profile may provide information about the pathology of the subject (e.g., if diseased), the abundance of an administered drug, or a drug by-product in the subject's serum, or the abundance of natural components whose levels might be affected by the treatment.

Drug Testing

The methods described herein can also be used to test the affinity of a test compound, e.g., a drug, for serum components. Information about whether a potential pharmaceutical interacts with a serum protein is useful for characterizing its efficacy and utility as a therapeutic agent.

For example, the test compound can be mixed with a biological sample, such as blood or serum or an at least partially purified preparation of a serum protein (e.g., a recombinant serum protein). After incubation, one or more serum proteins is isolated from the sample, e.g., using an affinity reagent, e.g., a reagent that includes a peptide ligand described herein. Binding of the test compound to the isolated serum protein can be determined, e.g., by quantifying the amount of test compound that is isolated with the serum protein. The test compound can be unlabeled or labeled (e.g., using a radioactive label or fluorescent label). A labeled test compound can be directly detected, e.g., using a scintillation proximity assay or fluorescence assay.

Unlabeled compounds can also be detected. For example, mass spectrometry can be used for detecting with unlabeled compounds. In another example, unlabeled compounds can be detected in a competition assay. Unlabeled compounds bound to the serum protein are separated from the serum protein and added to a binding reaction, e.g., in a well of a microtitre plate. The binding reaction includes an antibody that binds to the unlabeled compound and a known quantity of labeled compound. The amount of unlabeled compound present is determined by measuring the amount of labeled compound bound by the antibody. Competition by the unlabeled compound reduces the amount of bound labeled compound.

A related method includes administering the test compound to a subject, e.g., an animal model. After one or more appropriate intervals, a blood or serum sample is extracted from the subject. The amount of test compound associated with a serum protein can be determined, e.g., as described above. If the subject is a non-human mammal, an affinity ligand that is not species specific can be used.

Compounds that Modulate Interactions with a Serum Protein

It is also possible to screen for modulator compounds that modulate the interaction of a serum-protein binding compound and a serum protein, e.g., serum albumin. For example, it is possible to use a high throughput screen for compounds that disrupt (or enhance) the interaction between a naturally-occurring protein and serum protein.

One method for screening includes: contacting a candidate modulator compound to a complex that includes the serum protein-binding compound and the serum protein; and evaluating the interaction between the serum protein-binding compound and the serum protein. In one implementation, the serum protein is bound to an affinity reagent (e.g., a peptide ligand) and isolated. The isolated material is analyzed to determine the presence and/or amount of the serum protein-binding compound. A modulator compound that disrupts the interaction between the serum protein-binding compound and the serum protein may reduce or prevent isolation of the serum protein-binding compound.

A related method for screening involves contacting the serum protein to the candidate modulator compound, and subsequently adding the serum protein-binding compound to determine if the candidate modulator impairs or enhances the interaction between the serum protein and the serum protein-binding compound. Likewise, all three components can be combined together and then analyzed.

Identifying Binding Ligands for Serum Proteins.

Ligands that bind to a serum protein can be identified by a variety of methods including screening a display library. For example, phage display can be used to screen a library of linear or cyclic peptides for peptides that bind to a given serum protein. In addition, ligands that include an immunoglobulin domain, e.g., antibodies, can be generated (e.g., by immunization, or display library screening).

Peptide ligands that bind to human serum albumin and the Fc region of immunoglobulin are described herein and in U.S. provisional applications Ser. No. 60/331,352 filed Mar. 9, 2001, Ser. No. 60/292,975 filed May 23, 2001, Ser. No. 60/284,534, filed Apr. 18, 2001. Similarly, ligands can be isolated that bind to a serum protein such as: transferrin, α macroglobulins, ferritin, apolipoproteins, transthyretin, a protease inhibitor found in serum, retinol binding protein, thiostatin, α-fetoprotein, vitamin-D binding protein, or afamin.

One method of identifying a binding ligand for a serum protein is to screen a display library. A display library is a collection of entities; each entity includes an accessible polypeptide component and a recoverable component that encodes or identifies the polypeptide component. The polypeptide component can be of any length, e.g. from three amino acids to over 300 amino acids. In a selection, the polypeptide component of each member of the library is probed with the serum protein and if the polypeptide component binds to the protein, the display library member is identified, typically by retention on a support.

The screening of display libraries is advantageous, in that very large numbers (e.g., 5×10⁹) of potential binders can be tested, and successful binders isolated in a short period of time. Further, unlike immunization, ligands can be identified that bind to epitopes of serum proteins that are conserved among different species.

Retained display library members are recovered from the support and analyzed. The analysis can include amplification and a subsequent selection under similar or dissimilar conditions. For example, positive and negative selections can be alternated. The analysis can also include determining the amino acid sequence of the polypeptide component and purification of the polypeptide component for detailed characterization.

A variety of formats can be used for display libraries. Examples include the following.

Phage Display. One format utilizes viruses, particularly bacteriophages. This format is termed “phage display.” The polypeptide component is typically covalently linked to a bacteriophage coat protein. The linkage results form translation of a nucleic acid encoding the polypeptide component fused to the coat protein. The linkage can include a flexible peptide linker, a protease site, or an amino acid incorporated as a result of suppression of a stop codon. Phage display is described, for example, in Ladner et al., U.S. Pat. No. 5,223,409; Smith (1985) Science 228:1315-1317; WO 92/18619; WO 91/17271; WO 92/20791; WO 92/15679; WO 93/01288; WO 92/01047; WO 92/09690; WO 90/02809; de Haard et al. (1999) J. Biol. Chem 274:18218-30; Hoogenboom et al. (1998) Immunotechnology 4:1-20; Hoogenboom et al. (2000) Immunol Today 2:371-8; Fuchs et al. (1991) Bio/Technology 9:1370-1372; Hay et al. (1992) Hum Antibod Hybridomas 3:81-85; Huse et al. (1989) Science 246:1275-1281; Griffiths et al. (1993) EMBO J 12:725-734; Hawkins et al. (1992) J Mol Biol 226:889-896; Clackson et al. (1991) Nature 352:624-628; Gram et al. (1992) PNAS 89:3576-3580; Garrard et al. (1991) Bio/Technology 9:1373-1377; Rebar et al. (1996) Methods Enzymol. 267:129-49; Hoogenboom et al. (1991) Nuc Acid Res 19:4133-4137; and Barbas et al. (1991) PNAS 88:7978-7982.

Phage display systems have been developed for filamentous phage (phage fl, fd, and M13) as well as other bacteriophage (e.g. T7 bacteriophage and lambdoid phages; see, e.g., Santini (1998) J. Mol Biol. 282:125-135; Rosenberg et al. (1996) Innovations 6:1-6; Houshmet al. (1999) Anal Biochem 268:363-370). The filamentous phage display systems typically use fusions to a minor coat protein, such as gene III protein, and gene VIII protein, a major coat protein, but fusions to other coat proteins such as gene VI protein, gene VII protein, gene IX protein, or domains thereof can also been used (see, e.g., WO 00/71694). In a preferred embodiment, the fusion is to a domain of the gene III protein, e.g., the anchor domain or “stump,” (see, e.g., U.S. Pat. No. 5,658,727 for a description of the gene III protein anchor domain).

The valency of the polypeptide component can also be controlled. Cloning of the sequence encoding the polypeptide component into the complete phage genome results in multivariant display since all replicates of the gene III protein are fused to the polypeptide component. For reduced valency, a phagemid system can be utilized. In this system, the nucleic acid encoding the polypeptide component fused to gene III is provided on a plasmid, typically of length less than 700 nucleotides. The plasmid includes a phage origin of replication so that the plasmid is incorporated into bacteriophage particles when bacterial cells bearing the plasmid are infected with helper phage, e.g. M13K01. The helper phage provides an intact copy of gene III and other phage genes required for phage replication and assembly. The helper phage has a defective origin such that the helper phage genome is not efficiently incorporated into phage particles relative to the plasmid that has a wild type origin.

Bacteriophage displaying the polypeptide component can be grown and harvested using standard phage preparatory methods, e.g. PEG precipitation from growth media.

After selection of individual display phages, the nucleic acid encoding the selected polypeptide components, by infecting cells using the selected phages. Individual colonies or plaques can be picked, the nucleic acid isolated and sequenced.

Cell-based Display. In still another format the library is a cell-display library. Proteins are displayed on the surface of a cell, e.g., a eukaryotic or prokaryotic cell. Exemplary prokaryotic cells include E. coli cells, B. subtilis cells, spores (see, e.g., Lu et al. (1995) Biotechnology 13:366). Exemplary eukaryotic cells include yeast (e.g., Saccharomyces cerevisiae, Schizosaccharomyces pombe, Hanseula, or Pichia pastoris). Yeast surface display is described, e.g., in Boder and Wittrup (1997) Nat. Biotechnol. 15:553-557 and WO03029456. This application describes a yeast display system that can be used to display immunoglobulin proteins such as Fab fragments, and the use of mating to generate combinations of heavy and light chains.

In one embodiment, variegate nucleic acid sequences are cloned into a vector for yeast display. The cloning joins the variegated sequence with a domain (or complete) yeast cell surface protein, e.g., Aga2, Aga1, Flo1, or Gas1. A domain of these proteins can anchor the polypeptide encoded by the variegated nucleic acid sequence by a transmembrane domain (e.g., Flo1) or by covalent linkage to the phospholipid bilayer (e.g., Gas1). The vector can be configured to express two polypeptide chains on the cell surface such that one of the chains is linked to the yeast cell surface protein. For example, the two chains can be immunoglobulin chains.

Ribosome Display. RNA and the polypeptide encoded by the RNA can be physically associated by stabilizing ribosomes that are translating the RNA and have the nascent polypeptide still attached. Typically, high divalent Mg²⁺ concentrations and low temperature are used. See, e.g., Mattheakis et al. (1994) Proc. Natl. Acad. Sci. USA 91:9022 and Hanes et al (2000) Nat Biotechnol. 18:1287-92; Hanes et al. (2000) Methods Enzymol. 328:404-30. and Schaffitzel et al. (1999) J Immunol Methods. 231(1-2):119-35.

Peptide-Nucleic Acid Fusions. Another format utilizes peptide-nucleic acid fusions. Polypeptide-nucleic acid fusions can be generated by the in vitro translation of mRNA that include a covalently attached puromycin group, e.g., as described in Roberts and Szostak (1997) Proc. Natl. Acad. Sci. USA 94:12297-12302, and U.S. Pat. No. 6,207,446. The mRNA can then be reverse transcribed into DNA and crosslinked to the polypeptide.

Other Display Formats. Yet another display format is a non-biological display in which the polypeptide component is attached to a non-nucleic acid tag that identifies the polypeptide. For example, the tag can be a chemical tag attached to a bead that displays the polypeptide or a radiofrequency tag (see, e.g., U.S. Pat. No. 5,874,214).

Scaffolds. Scaffolds for display can include: antibodies (e.g., Fab fragments, single chain Fv molecules (scFV), single domain antibodies, camelid antibodies, and camelized antibodies); T-cell receptors; MHC proteins; extracellular domains (e.g., fibronectin Type III repeats, EGF repeats); protease inhibitors (e.g., Kunitz domains, ecotin, BPTI, and so forth); TPR repeats; trifoil structures; zinc finger domains; DNA-binding proteins; particularly monomeric DNA binding proteins; RNA binding proteins; enzymes, e.g., proteases (particularly inactivated proteases), RNase; chaperones, e.g., thioredoxin, and heat shock proteins; and intracellular signaling domains (such as SH2 and SH3 domains).

Another useful type of scaffolding domain is the immunoglobulin (Ig) domain. Methods using immunoglobulin domains for display are also known (see, e.g., Haard et al. (1999) J. Biol. Chem 274:18218-30; Hoogenboom et al. (1998) Immunotechnology 4:1-20. and Hoogenboom et al. (2000) Immunol Today 21:371-8).

Synthetic Peptides. The binding ligand can include a synthetic peptide, e.g., an artificial peptide of 30 amino acids or less. The synthetic peptide can include one or more disulfide bonds. Other synthetic peptides, so-called “linear peptides,” are devoid of cysteines. Synthetic peptides may have little or no structure in solution (e.g., unstructured), heterogeneous structures (e.g., alternative conformations or “loosely structured), or a singular native structure (e.g., cooperatively folded). Some synthetic peptides adopt a particular structure when bound to a target molecule. Some exemplary synthetic peptides are so-called “cyclic peptides” that have at least disulfide bond, and, for example, a loop of about 4 to 12 non-cysteine residues.

Peptide sequences that bind a molecular target are selected from a phage-display library. After identification, such peptides can be produced synthetically or by recombinant means. The sequences can be incorporated (e.g., inserted, appended, or attached) into longer sequences.

Exemplary Phage Display Libraries for Identifying Binding Peptides

Display libraries exhibiting variegated heterologous peptides on the surface of recombinant phage or other genetic packages (bacteria, yeast, other host cells) may be prepared in any of several ways known in the art. See, e.g., Kay et al., Phage Display of Peptides and Proteins: A Laboratory Manual (Academic Press, Inc., San Diego 1996) and U.S. Pat. No. 5,223,409 (Ladner et al.).

The following are six exemplary phage libraries that can be screened to find at least some of the polypeptide ligands described herein. Each library displays a short, variegated exogenous peptide on the surface of M13 phage. The peptide display of five of the libraries was based on a parental domain having a segment of 4, 5, 6, 7, 8, or 10 amino acids, respectively, flanked by cysteine residues. The pairs of cysteines are believed to form stable disulfide bonds, yielding a cyclic display peptide. The cyclic peptides are displayed at the amino terminus of protein III on the surface of the phage. The libraries were designated TN6/6, TN8/9, TN9/4, TN10/9, and TN12/1. A phage library with a 20-amino acid linear display was also screened; this library was designated Lin20.

The TN6/6 library was constructed to display a single cyclic peptide contained in a 12-amino acid variegated template. The TN6/6 library utilized a template sequence of Xaa₁-Xaa₂-Xaa₃-Cys₄-Xaa₅-Xaa₆-Xaa₇-Xaa₈-Cys₉-Xaa₁₀-Xaa₁₁-Xaa₁₂ (SEQ ID NO: 21), where each variable amino acid position in the amino acid sequence of the template is indicated by a subscript integer. Each variable amino acid position (Xaa) in the template was varied, independently, to permit the following substitutions: residues Xaa₁ and Xaa₁₂ were varied to contain any of the following 14 amino acids: Ala, Asp, Phe, Gly, His, Leu, Asn, Pro, Gln, Arg, Ser, Val, Trp, and Tyr; and residues Xaa₂, Xaa₃ Xaa₅, Xaa₆, Xaa₇, Xaa₈, Xaa₁₀, and Xaa₁₁ were independently varied to contain any of the common α-amino acids, except cysteine (Cys). The number of potential designed sequences is 3.3×10¹²; 2.0×10⁸ independent transformants were included in the library.

The TN8/9 library was constructed to display a single binding loop contained in a 14-amino acid template. The TN8/9 library utilized a template sequence of Xaa₁-Xaa₂-Xaa₃-Cys-Xaa₅-Xaa₆-Xaa₇-Xaa₈-Xaa₉-Xaa₁₀-Cys-Xaa₁₂-Xaa₁₃-Xaa₁₄ (SEQ ID NO: 25). The amino acids at position 1, 2, 3, 5, 6, 7, 8, 9, 10, 12, 13, and 14 in the template were varied to permit any amino acid except cysteine (Cys).

The TN9/4 library was constructed to display a single binding loop contained in a 15-amino acid template. The TN9/4 library utilized a template sequence Xaa₁-Xaa₂-Xaa₃-Cys-Xaa₅-Xaa₆-Xaa₇-Xaa₈ Xaa₉-Xaa₁₀-Xaa₁₁-Cys-Xaa₁₃-Xaa₁₄-Xaa₁₅ (SEQ ID NO: 424 The amino acids at position 1, 2, 3, 5, 6, 7, 8, 9, 10, 11, 13, 14 and 15 in the template were varied to permit any amino acid except cysteine (Cys).

The TN10/9 library was constructed to display a single cyclic peptide contained in a 16-amino acid variegated template. The TN10/9 library utilized a template sequence Xaa₁-Xaa₂-Xaa₃-Cys₄-Xaa₅-Xaa₆-Xaa₇-Xaa₈-Xaa₉-Xaa₁₀-Xaa₁₁-Xaa₂-Cys₁₃-Xaa₁₄-Xaa₁₅-Xaa₁₆ (SEQ ID NO: 22), where each variable amino acid position in the amino acid sequence of the template is indicated by a subscript integer. Each variable amino acid position (Xaa) was varied independently to permit the following substitutions. The amino acid positions Xaa₁, Xaa₂, Xaa₁₅ and Xaa₁₆ of the template were varied, independently, to permit each of the amino acids selected from a group of ten amino acids consisting of Asp, Phe, His, Leu, Asn, Pro, Arg, Ser, Trp, and Tyr; the amino acids at amino acid positions Xaa₃ and Xaa₁₄ in the template were varied, independently, to permit each amino acid selected from the group of fourteen amino acids consisting of Ala, Asp, Glu, Phe, Gly, His, Leu, Asn, Pro, Arg, Ser, Val, Trp, and Tyr; the amino acids at amino acid positions Xaa₅, Xaa_{6,} Xaa₇, Xaa₈, Xaa₉, Xaa₁₀, Xaa₁₁ and Xaa₁₂ (i.e., between the invariant cysteine residues at positions 4 and 13 in the template) were varied, independently, to permit each of the common α-amino acids, except cysteine. The number of potential designed sequences is 3.0×10¹⁶; and about 2.5×10⁸ independent transformants were included in the library.

The TN12/1 library was constructed to display a single cyclic peptide contained in an 18-amino acid template. The TN12/1 library utilized a template sequence Xaa₁-Xaa₂-Xaa₃-Cys₄-Xaa₅-Xaa₆-Xaa₇-Xaa₈-Xaa₉-Xaa₁₀-Xaa₁₁-Xaa₁₂-Xaa₁₃-Xaa₁₄-Cys₁₅-Xaa₁₆-Xaa₁₇-Xaa₁₈ (SEQ ID NO: 23), where each variable amino acid position in the amino acid sequence of the template is indicated by a subscript integer. The amino acid positions Xaa₁, Xaa₂, Xaa₁₇ and Xaa₁₈ of the template were varied, independently, to permit each amino acid selected from the group of 12 amino acids consisting of Ala, Asp, Phe, Gly, His, Leu, Asn, Pro, Arg, Ser, Trp, and Tyr. The amino acid positions Xaa₃, Xaa₅, Xaa_{6,} Xaa₇, Xaa₈, Xaa₉, Xaa₁₀, Xaa₁₁, Xaa₁₂, Xaa₁₃, Xaa₁₄, Xaa₁₆, of the the template were varied, independently, to permit each of the common (α-amino acids, except cysteine.

The Lin20 library was constructed to display a single linear peptide in a 20-amino acid template. The amino acids at each position in the template were varied to permit any amino acid except cysteine (Cys).

The techniques discussed in Kay et al., Phage Display of Peptides and Proteins: A Laboratory Manual (Academic Press, Inc., San Diego 1996) and U.S. Pat. No. 5,223,409 are useful for preparing a library of potential binders corresponding to the selected parental template. The libraries described above can be prepared according to such techniques, and screened for binding peptides against a serum protein, either immobilized on a solid surface or free in solution.

Screening Phage Display Libraries for Serum Protein Binding Peptides

In a typical screen, a phage library is contacted with and allowed to bind the target compound, e.g., a serum protein of interest, or a particular fragment or subcomponent thereof. To facilitate separation of binders and non-binders in the screening process, it is often convenient to immobilize the target compound on a solid support, although it is also possible to first permit binding to the target compound in solution and then segregate binders from non-binders by coupling the target compound to a support. By way of illustration, when incubated in the presence of the target, phage bearing a target-binding moiety form a complex with the target compound immobilized on a solid support whereas non-binding phage remain in solution and may be washed away with buffer. Bound phage may then be liberated from the target by a number of means, such as changing the buffer to a relatively high acidic or basic pH (e.g., pH 2 or pH 10), changing the ionic strength of the buffer, adding denaturants, or other known means.

For example to identify HSA-binding ligands, HSA can be adsorbed (by passive immobilization) to a solid surface, such as the plastic surface of wells in a multi-well assay plate, and then an aliquot of a phage display library was added to a well under appropriate conditions that maintain the structure of the immobilized HSA and the phage, such as pH 6-7. Phage in the libraries that display peptide loop structures that bind the immobilized HSA are retained bound to the HSA adhering to the surface of the well and non-binding phage can be removed. Phage bound to the immobilized HSA may then be eluted by washing with a buffer solution having a relatively strong acid pH (e.g., pH 2) or an alkaline pH (e.g., pH 8-9). The solutions of recovered phage that are eluted from the HSA are then neutralized and may, if desired, be pooled as an enriched mixed library population of phage displaying serum albumin binding peptides. Alternatively the eluted phage from each library may be kept separate as a library-specific enriched population of HSA binders. Enriched populations of phage displaying serum albumin binding peptides may then be grown up by standard methods for further rounds of screening and/or for analysis of peptide displayed on the phage and/or for sequencing the DNA encoding the displayed binding peptide.

One of many possible alternative screening protocols uses HSA target molecules that are biotinylated and that can be captured by binding to streptavidin, for example, coated on particles. As is described in an example below, phage displaying HSA binding peptides were selected from a library in such a protocol in which phage displaying HSA binding peptides were bound to a caprylate-biotinylated-HSA in solution at pH 7.4 in phosphate buffered saline (PBS) supplemented with 0.1% Tween 20 nonionic detergent and also 0.1 % sodium caprylate, which is known to stabilize HSA against temperature-induced denaturation and proteolytic attack. The caprylate-biotinylated-HSA/phage complexes in solution were then captured on streptavidin-coated magnetic beads. Phage were subsequently eluted from the beads for further study.

Recovered phage may then be amplified by infection of bacterial cells, and the screening process may be repeated with the new pool of phage that is now depleted in non-HSA binders and enriched in HSA binders. The recovery of even a few binding phage is sufficient to carry the process to completion. After a few rounds of selection, the gene sequences encoding the binding moieties derived from selected phage clones in the binding pool are determined by conventional methods, revealing the peptide sequence that imparts binding affinity of the phage to the target. An increase in the number of phage recovered after each round of selection and the recovery of closely related sequences indicate that the screening is converging on sequences of the library having a desired characteristic.

After a set of binding polypeptides is identified, the sequence information may be used to design other, secondary libraries, biased for members having additional desired properties.

Display technology can also be used to obtain ligands, e.g., antibody ligands or peptide ligands, that bind to particular epitopes of a target. This can be done, for example, by using competing non-target molecules that lack the particular epitope or are mutated within the epitope, e.g., with alanine. Such non-target molecules can be used in a negative selection procedure as described below, as competing molecules when binding a display library to the target, or as a pre-elution agent, e.g., to capture in a wash solution dissociating display library members that are not specific to the target.

The binding properties of a ligand that binds a serum protein can be readily assessed using various assay formats. For example, the binding property of a ligand can be measured in solution by fluorescence anisotropy, which provides a convenient and accurate method of determining a dissociation constant (K_{D}) of a binding moiety for a serum albumin from one or more different species. In one such procedure, a binding moiety described herein is labeled with fluorescein. The fluorescein-labeled binding moiety may then be mixed in wells of a multi-well assay plate with various concentrations of a particular species of serum albumin. Fluorescence anisotropy measurements are then carried out using a fluorescence polarization plate reader. The binding interaction between a serum protein and a non-covalently associated compound can be similarly characterized. Other solution measures for studying binding properties include fluorescence resonance energy transfer (FRET) and NMR.

Binding properties can also be characterized using a method wherein one binding partner is immobilized. Such methods include ELISA and surface plasmon resonance.

Serum Binding Protein Ligand Variants

It is also possible to use a variant of a serum binding protein ligand described herein or isolated by a method described herein. A number of variants are possible. A variant can be prepared and then tested, e.g., using a binding assay described above (such as fluorescence anisotropy). If the variant is function, it can be used as an affinity reagent to isolate a serum protein and associated compounds.

One type of variant is a truncation of a ligand described herein or isolated by a method described herein. In this example, the variant is prepared by removing one or more amino acid residues of the ligand can be removed from the N or C terminus. In some cases, a series of such variants is prepared and tested. Information from testing the series is used to determine a region of the ligand that is essential for binding the serum protein. A series of internal deletions or insertions can be similarly constructed and tested.

Another type of variant is a substitution. In one example, the ligand is subjected to alanine scanning to identify residues that contribute to binding activity. In another example, a library of substitutions at one or more positions is constructed. The library may be unbiased or, particularly if multiple positions are varied, biased towards an original residue.

A related type of variant is a ligand that includes one or more non-naturally occurring amino acids. Such variant ligands can be produced by chemical synthesis. One or more positions can be substituted with a non-naturally occurring amino acid. In some cases, the substituted amino acid may be chemically related to the original naturally occurring residue (e.g., aliphatic, charged, basic, acidic, aromatic, hydrophilic) or an isostere of the original residue.

It may also be possible to include non-peptide linkages and other chemical modification. For example, part or all of the ligand may be synthesized as a peptidomimetic, e.g., a peptoid (see, e.g., Simon et al. (1992) Proc. Natl. Acad. Sci. USA 89:9367-71 and Horwell (1995) Trends Biotechnol.13:132-4)

For example, variants of serum albumin-binding ligands (such as DX-321, DX-321-A, DX-321-B, DX-36, DX-236-A, and DX-236B) and immunoglobulin-binding ligands (such as DX249, DX249-A, DX253, DX-253-1, DX398, and DX398-A) can be used.

Sequences of Human Serum Proteins

The amino acid sequences of human serum proteins are well known and can be found in public sequence repositories, e.g., GenBank (National Center for Biotechnology Information, National Institutes of Health, Bethesda Md.). Further, in the human population, natural genetic variation can result in amino acid differences between serum proteins among individuals.

The following sequences are examples of at least some human serum protein amino acid sequences from particular individuals.

In many individuals, HSA has the amino acid sequence listed in SwissProt entry: P02768 and/or the following mature

| | |
|---|---|
| DAHKSEVAHRFKDLGEENFKALVLIAFAQYLQQCPFEDHVKLVNEVTEF | (SEQ ID NO:26) |
| AKTCVADESAENCDKSLHTLFGDKLCTVATLRETYGEMADCCAKQEPERNECFL | |
| QHKDDNPNLPRLVRPEVDVMCTAYHDNEETFLKKYLYELARRHPYFYAYELLFF | |
| AKRYKAAFTECCQAADKAACLLPKLDELRDEGKASSAKQRLKCASLQKFGERA | |
| FKAWAVARLSQRFPKAEFAEVSKLVTDLTKVHTECCHGDLLECADDRADLAKY | |
| ICENQDSISSKLKECCEKPLLEKSHCIAEVENDEMPADLPSLAADFVESKDVCKN | |
| YAEAKDVFLGMFLYEYARRHPDYSVVLLLRLAKTYETTLEKCCAAADPHECYA | |
| KVFDEFKPLVEEPQNLIKQNCELFEQLGEYKFQNALLVRYTKKVPQVSTPTLVEV | |
| SRNLGKVGSKCCKITPEAKRMPCAEDYLSVVLNQLCVLHEKTPVSDRVTKCCTE | |
| SLVNRRPCFSALEVDETYVPKEFNAETFTFHADICTLSEKERQIKKQTALVELVK | |
| HKPKATKEQLKAVMDDFAAFVEKCCKADDKETCFAEEGKKLVAASQAALGL. | |

Examples of human serum albumin variants include H27Q, H27Y, E106K, R122S, E378K, E400K, and E529K (numbered using the unprocessed sequence, wherein the initial D of SEQ ID NO: 26 corresponds to residue 25 of the unprocessed sequence).

Automated Methods and Information Management

Any and all aspects of the serum protein analysis platform can be automated. Automation, for example, can be used to process multiple different samples automatically. Liquid handling units can be used to isolate compounds associated with a serum protein from the sample and can automatically subject the isolated compounds to analytical methods such as electrophoresis and/or mass spectroscopy.

Equipment. Various robotic devices can be employed in the automation process. These include multi-well plate conveyance systems, magnetic bead particle processors, and liquid handling units. These devices can be built on custom specifications or purchased from commercial sources, such as Autogen (Framingham Mass.), Beckman Coulter (USA), Biorobotics (Woburn Mass.), Genetix (New Milton, Hampshire UK), Hamilton (Reno Nev.), Hudson (Springfield N.J.), Labsystems (Helsinki, Finland), Packard Bioscience (Meriden Conn.), and Tecan (Mannedorf, Switzerland).

Information Management. Information generated by the platform can be stored in a computer database (e.g., in digital form). Information, including information that describes the characterization of compounds associated with a serum protein, is stored in a central database. For example, the database can include information that describes a property of an associated compound (e.g., protein sequence, chemical structure, abundance, modification state, etc. and information that describes the sample (e.g., identity of its source, date, processing method, pathology, treatment, etc.). These items of information can be associated with each other. For example, a query about a particular state, e.g., a particular disease or treatment, can be used to identify properties of associated compounds found in that state. Likewise, a particular property of one or more associated compounds can be used as a query to identify states with which the property is prevalent.

The database can also include a profile, e.g., a description of a plurality of associated compounds from a sample in a particular state. Software can be used to compare profiles, e.g., to evaluate a given sample by comparison to the collection of profiles. One or more similar profiles can be used to infer information about the sample (e.g., to generate a diagnosis).

The database server can also be configured to communicate with each device using commands and other signals that are interpretable by the device. The computer-based aspects of the system can be implemented in digital electronic circuitry, or in computer hardware, firmware, software, or in combinations thereof. An apparatus of the invention, e.g., the database server, can be implemented in a computer program product tangibly embodied in a machine-readable storage device for execution by a programmable processor; and method actions can be performed by a programmable processor executing a program of instructions to perform functions described herein by operating on input data and generating output. One non-limiting example of an execution environment includes computers running Windows NT 4.0 (Microsoft) or better or Solaris 2.6 or better (Sun Microsystems) operating systems

The database server can also be interface with a network (e.g., an intranet or the Internet). The server can receive queries from a remote system and send information (e.g., about a profile of compounds associated with a serum protein) to the requesting system.

A query can be used to filter the database to identify records that compare favorably with a given tolerance to the query. For example, a set of mass spectroscopy peaks can be used to formulate a query. The filter can locate (and optionally display) records that include one or more (e.g., all) the peaks that are present in the query.

Peptide Immobilization on NHS-Sepharose Resin

One method for producing a matrix having an immobilized peptide is as follows.

Five micromoles of each peptide were dissolved in DMSO in a minimal volume and then added to 1 ml of NHS-sepharose affinity chromatography resin (Amersham Pharmacia Biotech, Piscataway, N.J.), which had been washed once with dimethyl sulfoxide (DMSO). The immobilization reaction was initiated by the addition of diisopropylethylamine to 2% (vol/vol). After 4 hours of slow mixing on a shaker table at room temperature, the reaction was quenched by the addition of an equal volume of 0.5 M hydroxylamine, pH 8, in water. For those peptides with ivDde-protected internal lysines, the hydroxylamine quench treatment also removed the ivDde-protecting group. To allow for complete protecting group removal, the quenched reaction was allowed to incubate overnight at room temperature. Once quenched and deprotected, the immobilized peptide-Sepharose resin was washed at least 3 times with water to remove solvent and unbound peptide. Non-specifically bound peptide was eluted off the resin by washing the resin at least three times in 30 mM phosphoric acid, pH 2. Since the NHS-Sepharose resin surface becomes negatively charged after hydrolysis, an acidic wash neutralizes the surface and removes any peptides bound non-covalently to the surface via electrostatic interactions. After washing, the resin was resuspended in water as a 50% v/v mixture. A 50 μl aliquot was used to determine the ligand density on the resin by quantitative amino acid analysis. Finally, the resin slurry was packed into 0.35 ml OMNIFIT™ glass columns (3 mm×50 mm) for analytical testing.

For larger preparative columns, the amounts of peptide and Sepharose were scaled up proportionally, and the final peptide Sepharose batches were packed into larger 10 ml Omnifit columns (10 mm diameter).

### EXAMPLE 1

Purification of HSA from Serum

A human serum sample was contacted to an HSA-binding peptide matrix that included the peptide ligands DX-236 and DX-321. The matrix was washed extensively with PBS, and eluted with a basic solution, 100 mM Tris, pH 9.1. Eluted material was rapidly neutralized with a buffer. An aliquot of this material was analyzed. FIG. 1 is a 2-D gel that separates this material.

Another aliquot of this material can be contacted to a chromatography resin that includes activated maleimide. The fraction that does not react with the resin may include compounds that dissociate from HSA during the elution at pH 9.1. The maleimide reacts with the free cysteine on HSA and covalently couples the HSA to the resin. The resin is treated with denaturants (e.g., 8M urea), and the eluant is collected and analyzed.

In particular the eluant can be separated by 2-D electrophoresis by pI and apparent molecular weight. The 2-D gel is stained and individual protein-staining spots are excised, digested with protease, and analyzed by MALDI-MS. The analysis of each spot is stored in a database.

### EXAMPLE 2

Purification of HSA from Serum

HSA was purified from blood serum using a preparative DX-236-Sepharose column (10 ml, 0.3 μmol/ml). Both the column and the serum sample were exchanged into 3 mM sodium phosphate, 20 mM NaCl, 0.1% Tween-20, pH 6.2. The 20 mM NaCl was added to the binding buffer to minimize nonspecific protein binding to the column. A 100 μl aliquot (approximately 5 mg HSA) was applied to the DX-236-Sepharose column previously equilibrated in the same buffer used for dialysis. A salt gradient between 20 and 44 mM was run, and then HSA was eluted with 100 mM Tris, pH 9.1. The results of the purification process are shown in Table 3.

TABLE 3
Purification of HSA Using DX-236 Sepharose Affinity Column

| Fraction | μg HSA | % Initial |
|---|---|---|
| Initial Load | 4805 | 100 |
| Flowthrough | 565 | 12 |
| Wash/Gradient | 88 | 1.8 |
| Elution | 4003 | 83 |
| Total | 4656 | 96.8 |

As shown in Table 6, the column bound essentially all the HSA in a 0.1 ml serum injection (˜5 mg HSA total) and released essentially all the bound HSA with a 100 mM Tris, pH 9.1 wash (Table 6).

### EXAMPLE 3

HSA-binding Matrices

DX-232, DX-236, and DX-321 binding peptides were used for affinity chromatography development. Each peptide was immobilized at high density on NHS-Sepharose resin using the procedure outlined above. The peptides were immobilized via a C-terminal lysine. As determined by quantitative amino acid analysis, the ligand densities for DX-321 -Sepharose, DX-236-Sepharose, and DX-232-Sepharose columns were 3.2, 0.8, and 2.4 μmol/ml, respectively. Each column was tested for HSA binding (1 mg injection) in binding buffer—3 mM sodium phosphate, 0.1% Tween-20 detergent, pH 6.2. Since some of the peptides showed a sharp increase in K_{D} as the pH was increased to 9.1, a 100 mM Tris, pH 9.1 buffer can be used to elute HSA from these columns.

For analytical affinity column testing, albumin was dissolved at 1 mg/ml concentration in 3 mM sodium phosphate, pH 6.2, 0.01% Tween-20 non-ionic detergent (equilibration buffer). One milliliter of albumin solution was passed through each column (0.35 ml) previously equilibrated in equilibration buffer. The columns were washed with the same equilibration buffer and then eluted with 100 mM Tris, pH 9.1 (flow rate, 0.5 ml/min for all steps). The column chromatography was carried out using a BIO-RAD BIOLOGIC™ monitoring system (Hercules, Calif.) throughout this testing with absorbance monitoring at 280 nm.

For preparative DX-236-Sepharose affinity column (10 ml) testing, human serum was dialyzed against 3 mM phosphate, pH 6.2, 20 mM NaCl, 0.01% Tween-20 non-ionic detergent (equilibration buffer). One hundred microliters (100 μl) of dialyzed serum were injected onto the preparative DX-236-Sepharose chromatography column, which was previously equilibrated with buffer. The column was washed with the same buffer, followed by a gradient between 20 and 44 mM NaCl, and finally the HSA was eluted with 100 mM Tris, pH 9.1. For all steps, the flow rates were 5 ml/min.

Each column performed differently in the initial HSA binding tests. Although soluble peptide DX-232 bound HSA with the highest affinity, immobilized DX-232 on a sepharose column captured no detectable HSA. DX-236-Sepharose, on the other hand, was the best performer and quantitatively bound the entire 1 mg injection (total capacity ≧2.7 mg/ml) (see, Table 4, below).

TABLE 4
Analysis of HSA Affinity Columns

| Peptide in | | | | |
|---|---|---|---|---|
| Affinity | | | | Total |
| Column | Fraction | μg HSA | % Initial Load | Capacity |
| DX-321 | Flow through | 554 | 55.4 | |
| | Elution | 370 | 37.0 | >1.1 mg/ml |
| DX-236 | Flow through | 0 | 0 | |
| | Elution | 947 | 94.7 | >2.7 mg/ml |

At higher HSA loads, the same DX-236 column was capable of binding at least 4 mg HSA, which corresponds to a total capacity of greater than 11 mg/ml. DX-321-Sepharose was an intermediate performer and bound a fraction of the total material (total capacity >1.1 mg/ml). The Tris elution buffer eluted all of the bound HSA from both DX-236- and DX-321-Sepharose columns.

### EXAMPLE 4

Species Specificity of Isolated HSA Binders

To test the binding specificity of DX-236 and DX-321 for HSA over other albumins, their dissociation constants (K_{D}) were determined against a panel of mammalian albumins both in 3 mM sodium phosphate, pH 6.2, and in PBS (10 mM sodium phosphate, 140 mM NaCl, pH 7.4). The results are set forth in Table 5.

TABLE 5
Species Specificity Data for Affinity Columns

| | | | DX-236 | DX-236 | DX-321 | DX-321 |
|---|---|---|---|---|---|---|
| | | | phosphate, | PBS, | phosphate, | PBS, |
| | | | pH 6.2, | pH 7.4, | pH 6.2, | pH 7.4, |
| | | % Identity | O M NaCl | 0.14 M NaCl | O M NaCl | 0.14 M NaCl |
| Species | pI | to Human | K_{D} (μM) | K_{D} (μM) | K_{D} (μM) | K_{D} (μM) |
| Human | 5.67 | 100 | 1.9 | 11.0 | 0.9 | 84 |
| Rhesus | 5.67 | 93.2 | 1.1 | 23 | 38 | 82 |
| Bovine | 5.60 | 75.6 | 1.1 | 13.3 | 21 | >200 |
| Goat | N.D. | N.D. | 1.6 | 23 | 95 | 83 |
| Pig | 5.75 | 75.0 | 0.5 | 12 | 21 | >200 |
| Rabbit | 5.65 | 75.0 | 0.5 | 18 | 32 | >200 |
| Rat | 5.80 | 73.2 | 1.6 | 25 | 23 | 117 |
| Mouse | 5.53 | 72.0 | 5.5 | 32 | >200 | >200 |
| Chicken | 5.19 | N.D. | >200 | >200 | >200 | >200 |
| (egg) | | | | | | |

N.D. = not determined

In the 3 mM phosphate, pH 6.2 buffer, labeled DX-236 bound to all the albumins tested with high affinity, except for murine serum albumin (MSA). In PBS, the same affinity trend appeared with DX-236, except all the K_{D} values were higher than for the low salt, pH 6.2 condition.

Labeled DX-321 bound each mammalian albumin with a substantially higher K_{D} compared to HSA in the low salt, pH 6.2 buffer. In particular, MSA bound DX-321 with a K_{D} greater than 200 μM compared to HSA, which bound DX-321 with a submicromolar K_{D}. All of the other non-human albumins also bound weakly to DX-321 and had K_{D} values at least 10 times greater than for HSA. In PBS, however, the DX-321 affinity differences between HSA and the others were less pronounced compared to the pH 6.2 results. As a negative control, each peptide (DX-236 and DX-321) was also tested for binding to chicken ovalbumin in both sets of buffers and found that neither peptide showed any significant binding (Table 4). Chicken ovalbumin is not homologous to HSA as determined by sequence alignment analysis. This analysis indicated that immobilized DX-236 can be used to purify other mammalian albumins.

To demonstrate this property, the same DX-236- and DX-321-Sepharose columns were tested against bovine serum albumin (BSA), goat serum albumin (GSA), and murine serum albumin (MSA) in the pH 6.2 buffer. One mg of each type of albumin was injected onto each column (0.35 ml) previously equilibrated in 3 mM Phosphate, pH 6.2, 0.01% Tween-20. The columns were washed with equilibration buffer and then eluted with 100 mM Tris, pH 9.1 (flow rate, 1 ml/min). As shown in Table 6 below, DX-236-Sepharose quantitatively captured all three albumins like HSA.

TABLE 6
Mammalian Serum Albumin Testing with DX-236 and DX-321

| | | DX-236 Column | | DX-321 Column | |
|---|---|---|---|---|---|
| Albumin | Protein Load | FT (mg) | Elution (mg) | FT (mg) | Elution (mg) |
| Bovine | 1 mg | 0 | 0.72 | 0.86 | 0.15 |
| Goat | 1 mg | 0 | 0.79 | 0.93 | 0.11 |
| Mouse | 0.5 mg | 0.05 | 0.59 | 0.49 | 0.13 |

FT = flowthrough

DX-236-Sepharose can be used as a “pan-albumin” binder for the affinity purification of nearly any mammalian albumin from serum. These results indicate that DX-236 could also be used to deplete albumin from serum samples prior to other analyses.

The data in Table 6 also show that DX-321-Sepharose captures the three non-human albumins poorly, as is expected based on the solution affinity data shown in Table 4. Of the three non-human albumins, BSA was captured most effectively by the DX-321-Sepharose resin. About 15% of the BSA present in the starting material was captured and subsequently eluted under the same chromatography conditions that allowed quantitative capture of DX-236-Sepharose resin. Goat serum albumin (GSA) and mouse serum albumin (MSA) were even less effectively captured by the DX-321-Sepharose column than with BSA. Thus, the DX-321-Sepharose column may be advantageously used to purify HSA from solutions containing non-human serum albumins.

### EXAMPLE 5

Immunoglobulin Binding Peptides

Dissociation constants were determined for the following immunoglobulin-binding peptides, which were prepared using the Fc-region binding peptides of SEQ ID NOS: B57, B58, B108, B115, B124, and B143, respectively:

| | |
|---|---|
| Ac-AGSYWCKIWDVCPQSPGPEGGGK-NH₂; | (SEQ ID NO:371, designated DX392) |
| Ac-AGKYWCNLWGVCPANPGPEGGGK-NH₂ ; | (SEQ ID NO:372, designated DX395) |
| Ac-AGTYWCTFWELPCDPAPGPEGGGK-NH₂ ; | (SEQ ID NO:373, designated DX404) |
| Ac-AGPHNCDDHYWYCKWFPGPEGGGK-NH₂ ; | (SEQ ID NO:374, designated DX389) |
| Ac-AGAATCSTSYWYYQWFCTDSPGPEGGGK-NH₂ ; and | (SEQ ID NO:375, designated DX398) |
| Ac-AGYWYCWFPDRPECPLYPGPEGGGK-NH₂ . | (SEQ ID NO:376, designated DX413) |

Peptides were synthesized by BACHEM and then Oregon Green labeled and HPLC purified. Binding studies were performed using human plasma IgG isoforms: IgG1, IgG2, IgG3, and IgG4, obtained from Calbiochem.

Binding studies were carried out at either pH 4.0, 7.5, or 9.5, with or without salt in the following buffers:

1) 10 mM Sodium Citrate, 0.01 % Tween 20, pH 4.0;

2) 10 mM Sodium Citrate, 500 mM Sodium Chloride, 0.01 % Tween 20, pH 4.0;

3) 10 mM Tris-HCl, 0.01 % Tween 20, pH 7.5;

4) 10 mM Tris-HCl, 500 mM Sodium Chloride, 0.01 % Tween 20, pH 7.5;

5) 10 mM Sodium Bicarbonate, 0.01 % Tween 20, pH 9.5;

6) 10 mM Sodium Bicarbonate, 500 mM Sodium Chloride, 0.01 % Tween 20, pH 9.5; or

7) TBS, 0.01 % Tween 20, pH 7.5.

Results of the binding studies are shown in Table 7.

TABLE 7
Summary of KD values for the IgG binding Oregon Green Labeled Peptides

| | | K_{D}(μM) | | | | | | |
|---|---|---|---|---|---|---|---|---|
| | IgG | pH 4.0 − | pH 4.0 + | pH 7.5 − | pH 7.5 + | pH 9.5 − | pH 9.5 + | |
| Peptide | isoform | salt | salt | salt | salt | salt | salt | TBS |
| DX389 | IgG1 | nb* | nb | nb | nb | nb | nb | nb |
| | IgG2 | nb | nb | nb | binds^{Φ} | nb | nb | nb |
| | IgG3 | 2.5 ± 1.0 | 1.8 ± 1.4 | nb | binds | nb | nb | nb |
| | IgG4 | nb | nb | nb | nb | nb | nb | nb |
| DX392 | IgG1 | nb | nb | nb | nb | nb | nb | nb |
| | IgG2 | nb | nb | nb | nb | nb | nb | nb |
| | IgG3 | 0.32 ± 0.08 | 0.6 ± 0.2 | nb | binds | nb | binds | nb |
| | IgG4 | nb | nb | nb | nb | nb | nb | nb |
| DX395 | IgG1 | nb | nb | nb | nb | nb | nb | nb |
| | IgG2 | nb | nb | nb | nb | nb | nb | nb |
| | IgG3 | 1.0 ± 0.26 | 1.8 ± 1 | nb | 1.9 ± 0.9 | nb | binds | nb |
| | IgG4 | nb | nb | binds | nb | nb | nb | nb |
| DX398 | IgG1 | 2.4 ± 3.3 | nb | 4.6 ± 1.2 | nb | nb | nb | nb |
| | IgG2 | 1.8 ± 1.2 | nb | nb | nb | binds | nb | nb |
| | IgG3 | 0.02 ± 1.0 | 0.04 ± 0.01 | nb | 0.3 ± 0.03 | binds | 0.3 ± 0.1 | binds |
| | IgG4 | 1.6 ± 1.5 | nb | 3.5 ± 0.8 | nb | nb | nb | nb |
| DX404 | IgG1 | 1.5 ± 0.8 | 2.0 ± 1.7 | 8.8 ± 4.0 | nb | nb | nb | nd |
| | IgG2 | 1 ± 0.4 | 2.0 ± 1 | 8.6 +3.5 | nb | nb | nb | nd |
| | IgG3 | 0.01 ± 0.01 | 0.20 ± 0.06 | 11 ± 5.4 | 3.7 ± 0.8 | nb | binds | nd |
| | IgG4 | nb | nb | nb | nb | nb | nb | nd# |
| DX413 | IgG1 | nb | nb | nb | nb | nb | nb | nd |
| | IgG2 | nb | nb | nb | nb | nb | nb | nd |
| | IgG3 | 0.84 ± 0.08 | 1.1 ± 0.2 | nb | nb | nb | nb | nd |
| | IgG4 | nb | nb | nb | nb | nb | nb | nd |

*nb: no significant binding observed
^{Φ}binds: peptide appears to bind but the signal change could not be fit to obtain a reliable estimate of the K_{D} The K_{D} is estimated to be greater than 10 μM.
#nd: not determined.

The results shown in Table 7 demonstrate that DX389 specifically binds IgG3 at pH 4.0 in either the presence or absence of salt with moderate affinity (K_{D}≅2 μM). This interaction was not observed in the presence or absence of salt either at pH 7.5 or 9.5.

Peptide DX392 bound IgG3 specifically at pH 4.0 both in the presence and absence of salt and with a high affinity (K_{D}≅0.3-0.6 μM). This interaction was lower at pH 7.5 and pH 9.5 in the presence of salt and was not observed at either pH in the absence of salt.

Peptide DX395 bound IgG3 specifically at pH 4.0 in either the presence or absence of salt at moderate affinity (K_{D}≅1-2 μM). The affinity was approximately the same (K_{D}≅1.9 μM) in the presence of salt. This interaction was diminished at pH 9.5 in the presence of salt and was not observed at pH 7.5 or 9.5 in the absence of salt.

Peptide DX398 bound all four IgG isoforms at pH 4.0 in the absence of salt with moderate affinity (K_{D}≅2 μM) for IgG1, IgG2, and IgG4, and high affinity (K_{D}≅0.02 μM) for IgG3. At pH 4.0 in the presence of salt, peptide DX398 maintained a high affinity for IgG3 but did not interact with IgG, IgG2, or IgG4.

At pH 7.5, DX398 bound IgG1 and IgG4 only in the absence of salt and in the presence of salt, only bound IgG3. At pH 9.5, this peptide only bound IgG3 and the interaction was favored by increasing ionic strength.

Peptide DX404 bound IgG1 and IgG2 at pH 4.0 in the presence or absence of salt with moderate affinity (K_{D}≅2 μM) and had a higher affinity for IgG3 (K_{D}≅0,01 μM). In the presence of salt, the affinity for IgG3 increased to 0.2 μM. The affinity for IgG1 and IgG2 was reduced at pH 7.5 in the absence of salt and not observed in the presence of salt or at pH 9.5. IgG3 binding at pH 7.5 and 9.5 was favored in the presence of salt.

Peptide DX413 bound only to IgG3 at pH 4.0 in the presence or absence of salt with moderate affinity (K_{D}≅1.0 μM).

The data in Table 7 indicate that the peptides bind IgG with varying isoform specificities in a pH and salt-dependent manner. In general, the peptides in Table 8 can be grouped into two “classes” based on their specificity and mode of interaction:

Class 1 includes DX389, DX392, DX395 and DX413. Essentially these peptides all appear to exhibit primary specificity for IgG3. In addition, the interaction appears to be favored by low pH and high ionic strength. Binding is weakest at high pH and low salt.

Class 2 includes DX398. This peptide exhibits isoform specificity that is alterable by ionic strength. At low pH in the absence of salt, this peptide binds all IgG isoforms but in the presence of salt, it only binds IgG3 with very high affinity (K_{D}≅0.04-0.3 μM) at pH 4.0, 7.5, and 9.5 (See Table 7). DX404 is similar to DX398, however this peptide, unlike DX398, does not exhibit the salt-dependent IgG3 specificity at pH 4.0 but does exhibit salt-dependent IgG3 specificity at pH 7.5 and 9.5.

Other embodiments are within the following claims.

What is claimed:

1. A method of evaluating a sample, the method comprising:
providing a sample that comprises (i) a serum albumin, (ii) one or more compounds physically associated with the serum albumin and (iii) a serum albumin-binding agent that is free of an antigen-binding immunoglobulin variable domain;
allowing the serum albumin-binding agent to bind to the serum albumin to form a complex;
separating the complex from one or more components of the sample; and
evaluating one or more of the physically associated compounds.

2. A method of evaluating a sample, the method comprising:
providing a sample that comprises (i) a serum albumin, (ii) one or more compounds physically associated with the serum albumin and (iii) a serum albumin-binding agent that comprises a peptide that independently binds to serum albumin;
allowing the serum albumin-binding agent to bind to the serum albumin to form a complex;
separating the complex from one or more components of the sample; and
evaluating one or more of the physically associated compounds.

3. The method of claim 1 or 2 wherein the serum albumin-binding agent binds serum albumin with an affinity of less than 5 μM.

4. The method of claim 2 wherein the peptide is less than 30 amino acids in length.

5. The method of claim 2 wherein the peptide comprises an intra-molecular disulfide bond.

6. The method of claim 4 wherein the peptide comprises DX-236 or DX-321 or an amino acid sequence that differs from DX-236 or DX-321 by fewer than four amino acid substitutions.

7. The method of claim 1 wherein the serum albumin-binding agent is coupled to an insoluble support.

8. The method of claim 1 wherein the serum albumin-binding agent binds to serum albumin from a plurality of species.

9. The method of claim 1 wherein at least one of the evaluated physically associated compounds is non-covalently associated with the serum albumin.

10. The method of claim 9 further comprising separating one or more of the physically associated compounds from the serum albumin.

11. The method of claim 10 wherein the separating of one or more of the physically associated compounds from the serum albumin is prior to the evaluating.

12. The method of claim 1 or 2 further comprising separating the at least one non-covalently associated compounds from the serum albumin prior to the evaluating.

13. The method of claim 12 wherein the separating from the serum albumin comprises covalently attaching the serum albumin to an insoluble support.

14. The method of claim 13 wherein the covalent attachment is to a free cysteine of the serum albumin.

15. A method of evaluating a sample, the method comprising:
providing a sample that comprises (i) a serum albumin, (ii) one or more compounds physically associated with the serum albumin and (iii) a serum albumin-binding agent;
allowing the serum albumin-binding agent to bind to the serum albumin to form a complex;
separating the complex from one or more components of the sample;
covalently attaching the serum albumin to an insoluble matrix; and
separating at least one of the one or more compounds physically associated with the serum albumin from the serum albumin.

16. The method of claim 15 wherein the covalent attachment is to a free cysteine of the serum albumin.

17. The method of claim 15 further comprising evaluating one or more of the physically associated compounds that becomes separated from the serum albumin.

18. The method of claim 15 wherein the covalent attachment is formed using a thiol reactive group.

19. The method of claim 18 wherein the thiol reactive group comprises a halogen derivative.

20. The method of claim 19 wherein the thiol reactive group comprises iodoacetamide.

21. The method of claim 18 wherein the thiol reactive group comprises a maleimide.

22. The method of claim 18 wherein the thiol reactive group comprises a thiol exchange reagent.

23. The method of claim 22 wherein the thiol exchange reagent is a pyridyl disulfide.

24. The method of claim 15 wherein the separating comprises denaturing the serum albumin.

25. The method of claim 1 wherein at least one of the evaluated covalently associated compounds is non-proteinaceous.

26. The method of claim 1 wherein the evaluating comprises one or more of: gel electrophoresis, mass spectroscopy, chromatography, and protein sequencing.

27. The method of claim 1 wherein the evaluating comprises detecting a given compound using an affinity reagent specific for the given compound.

28. The method of claim 27 wherein the affinity reagent is an antibody.

29. The method of claim 1 wherein the evaluating comprises detecting a compound other than a fatty acid, hematin, and bilirubin.

30. The method of claim 1 wherein the evaluating comprises detecting a polypeptide.

31. The method of claim 1 wherein the evaluating comprises eluting an associated compound from the serum albumin by contacting the complex with a synthetic affinity ligand specific for an epitope on the serum albumin.

32. The method of claim 1 wherein the evaluating comprises eluting an associated compound by contacting the complex with a natural compound that binds to the serum albumin.

33. The method of claim 32 wherein the natural compound comprises a component selected from the group consisting of: a fatty acid, hematin, and bilirubin.

34. The method of claim 32 wherein the natural compound comprises a negatively charged aromatic group having a molecular weight of less than 500 Daltons.

35. The method of claim 1 wherein the serum albumin is a human serum albumin.

36. The method of claim 1 wherein the serum albumin is an artificial mutant of a naturally-occurring serum albumin.

37. The method of claim 1 further comprising digitally recording information that (i) indicates the presences or absence of a given compound among the evaluated one or more physically associated compounds, or (ii) describes the one or more physically associated compounds.

38. The method of claim 1 further comprising providing a second sample, and evaluating one or more of the physically associated compounds in the second sample.

39. The method of claim 38 further comprising comparing the results of evaluating the one or more of the physically associated compounds for the first sample to the second sample.

40. The method of claim 39 wherein the first sample is from a first subject, and the second sample is from a second subject.

41. The method of claim 40 wherein the first subject is treated with an agent, and the second subject is not treated with the agent.

42. The method of claim 40 wherein the first subject and second subject are subjected to different environmental conditions.

43. The method of claim 1 or 2 wherein the serum albumin-binding agent and the serum albumin preferentially dissociate in solutions above pH 8.

44. The method of claim 1 wherein the sample is obtained from a subject.

45. The method of claim 44 wherein the subject is a human.

46. The method of claim 45 wherein the sample comprises blood or serum.

47. The method of claim 45 wherein the sample is obtained from a biopsy.

48. The method of claim 45 wherein the sample is obtained from a tumor or a region within 5 mm of a tumor.

49. The method of claim 45 wherein the subject is treated with a therapeutic composition prior to obtaining the sample.

50. The method of claim 49 wherein one or more of the evaluated physically associated compounds is an endogenous compound.

51. The method of claim 49 wherein one or more of the evaluated physically associated compounds is a component of the therapeutic composition.

52. A method of evaluating a sample, the method comprising:
providing a sample that comprises (i) a soluble immunoglobulin protein that includes at least one immunoglobulin domain, (ii) one or more compounds physically associated with the immunoglobulin protein and (iii) immunoglobulin-binding agent that comprises a peptide that specifically binds to the immunoglobulin protein at a site other than an antigen binding site;
allowing the immunoglobulin-binding agent to bind to the soluble immunoglobulin protein to form a complex that includes one or more compounds physically associated with the soluble immunoglobulin protein;
separating the complex from one or more components of the sample; and
evaluating one or more of the physically associated compounds.

53. The method of claim 52 wherein the soluble immunoglobulin protein is a naturally-occurring protein.

54. The method of claim 52 wherein the soluble immunoglobulin protein is an IgG.

55. The method of claim 52 wherein the one or more physically associated compounds comprises an antigen.

56. The method of claim 52 wherein the sample is obtained from a subject having an infection.

57. The method of claim 52 wherein the sample is obtained from a subject having immunological disorder.

58. The method of claim 57 wherein the immunological disorder is an auto-immune disorder.

59. The method of claim 52 wherein the peptide is less than 30 amino acids in length.

60. The method of claim 52 wherein the peptide comprises an intra-molecular disulfide bond.

61. A method of evaluating a sample, the method comprising:
providing a sample that comprises (i) a soluble serum protein, (ii) one or more compounds physically associated with the soluble serum protein, and (iii) serum protein-binding agent that comprises a peptide that specifically binds to the serum protein;
allowing the serum protein-binding agent to bind to the soluble serum protein to form a complex that includes one or more compounds physically associated with the soluble serum protein;
separating the complex from one or more components of the sample; and
evaluating one or more of the physically associated compounds.

62. The method of claim 61 wherein the serum protein is serum albumin.

63. The method of claim 61 wherein the serum protein is at least 0.01% of the protein fraction in blood serum.

64. The method of claim 61 wherein the serum protein is selected from the group consisting of: transferrin, a macroglobulins, ferritin, apolipoproteins, transthyretin, a protease inhibitor found in serum, retinol binding protein, thiostatin, a-fetoprotein, vitamin-D binding protein, or afamin.

65. A method of mapping a physical interaction between serum albumin and an associated compound, the method comprising:
providing a complex comprising a serum albumin and an associated compound;
evaluating binding of a ligand (e.g., a peptide ligand described herein) to the complex, wherein the ligand binds to serum albumin with an affinity of less than 5 μM, the ligand is free of an immunoglobulin variable domain, and binding of the non-antibody ligand to the complex indicates that the associated compound does not bind an epitope that overlaps the epitope bound by the non-antibody ligand.

66. The method of claim 65 further comprising: evaluating binding of a ligand to the complex, wherein the second ligand binds to serum albumin with an affinity of less than 5 μM.

67. The method of claim 65 wherein one of the first and second non-antibody ligand binds is prevented from binding to the complex.


SEQUENCE LISTING (430 sequences)

SEQ ID NO: 1 - protein, 6 aa, Artificial Sequence
  Examplary motif
CXXXXC

SEQ ID NO: 2 - protein, 12 aa, Artificial Sequence
  Examplary motif
XXXCXXXXCXXX

SEQ ID NO: 3 - protein, 21 aa, Artificial Sequence
  Examplary motif
AEGTGSXXXCXXXXCXXXAPE

SEQ ID NO: 4 - protein, 12 aa, Artificial Sequence
  Examplary motif
CXXXXXXXXXXC

SEQ ID NO: 5 - protein, 18 aa, Artificial Sequence
  Examplary motif
XXXCXXXXXXXXXXCXXX

SEQ ID NO: 6 - protein, 27 aa, Artificial Sequence
  Examplary motif
AEGTGXXXXCXXXXXXXXXXCXXXXPE

SEQ ID NO: 7 - protein, 6 aa, Artificial Sequence
  example of serum albumin-binding agent
CTIFLC

SEQ ID NO: 8 - protein, 12 aa, Artificial Sequence
  example of serum albumin-binding agent
CEGKDMIDWVYC

SEQ ID NO: 9 - protein, 12 aa, Artificial Sequence
  example of serum albumin-binding agent
CDRIAWYPQHLC

SEQ ID NO: 10 - protein, 10 aa, Artificial Sequence
  example of serum albumin-binding agent
CEPWMLRFGC

SEQ ID NO: 11 - protein, 6 aa, Artificial Sequence
  example of serum albumin-binding agent
CDQWFC

SEQ ID NO: 12 - protein, 6 aa, Artificial Sequence
  example of serum albumin-binding agent
CNNALC

SEQ ID NO: 13 - protein, 6 aa, Artificial Sequence
  example of serum albumin-binding agent
CDHFFC

SEQ ID NO: 14 - protein, 6 aa, Artificial Sequence
  example of serum albumin-binding agent
CWHFSC

SEQ ID NO: 15 - protein, 12 aa, Artificial Sequence
  example of serum albumin-binding agent
CVTRWANRDQQC

SEQ ID NO: 16 - protein, 12 aa, Artificial Sequence
  example of serum albumin-binding agent
CVTDWANRHQHC

SEQ ID NO: 17 - protein, 12 aa, Artificial Sequence
  example of serum albumin-binding agent
CVKDWANRRRGC

SEQ ID NO: 18 - protein, 12 aa, Artificial Sequence
  example of serum albumin-binding agent
CKFSWIRSPAFC

SEQ ID NO: 19 - protein, 31 aa, Artificial Sequence
  serum albumin-binding agents
AEGTGDFWFCDRIAWYPQHLCEFLDPEGGGK

SEQ ID NO: 20 - protein, 27 aa, Artificial Sequence
  serum albumin-binding agents
AEGTGDRNMCKFSWIRSPAFCARADPE

SEQ ID NO: 21 - protein, 12 aa, Artificial Sequence
  template sequence
XXXCXXXXCXXX

SEQ ID NO: 22 - protein, 16 aa, Artificial Sequence
  template sequence
XXXCXXXXXXXXCXXX

SEQ ID NO: 23 - protein, 18 aa, Artificial Sequence
  template sequence
XXXCXXXXXXXXXXCXXX

SEQ ID NO: 24 - protein, 29 aa, Artificial Sequence
  modified serum albumin-binding agent
AEGTGDRNMCKFSWIRSPAFCARADPEXK

SEQ ID NO: 25 - protein, 14 aa, Artificial Sequence
  template sequence
XXXCXXXXXXCXXX

SEQ ID NO: 26 - protein, 585 aa, Homo sapiens [sequence omitted]

SEQ ID NO: 27 - protein, 18 aa, Artificial Sequence
  example of serum albumin-binding agents
ADFCEGKDMIDWVYCRLY

SEQ ID NO: 28 - protein, 18 aa, Artificial Sequence
  example of serum albumin-binding agents
FWFCDRIAWYPQHLCEFL

SEQ ID NO: 29 - protein, 18 aa, Artificial Sequence
  example of serum albumin-binding agents
DWDCVTRWANRDQQCWGP

SEQ ID NO: 30 - protein, 18 aa, Artificial Sequence
  example of serum albumin-binding agents
DWDCVTRWANRDQQCWAL

SEQ ID NO: 31 - protein, 18 aa, Artificial Sequence
  example of serum albumin-binding agents
DWDCVTDWANRHQHCWAL

SEQ ID NO: 32 - protein, 18 aa, Artificial Sequence
  example of serum albumin-binding agents
DWQCVKDWANRRRGCMAD

SEQ ID NO: 33 - protein, 18 aa, Artificial Sequence
  example of serum albumin-binding agents
RNMCKFSWIRSPAFCARA

SEQ ID NO: 34 - protein, 27 aa, Artificial Sequence
  example of serum albumin-binding agents
AEGTGDADFCEGKDMIDWVYCRLYDPE

SEQ ID NO: 35 - protein, 27 aa, Artificial Sequence
  example of serum albumin-binding agents
AEGTGDFWFCDRIAWYPQHLCEFLDPE

SEQ ID NO: 36 - protein, 27 aa, Artificial Sequence
  example of serum albumin-binding agents
AEGTGDDWDCVTRWANRDQQCWGPDPE

SEQ ID NO: 37 - protein, 27 aa, Artificial Sequence
  example of serum albumin-binding agents
AEGTGDDWDCVTRWANRDQQCWALDPE

SEQ ID NO: 38 - protein, 27 aa, Artificial Sequence
  example of serum albumin-binding agents
AEGTGDDWDCVTDWANRHQHCWALDPE

SEQ ID NO: 39 - protein, 27 aa, Artificial Sequence
  example of serum albumin-binding agents
AEGTGDDWQCVKDWANRRRGCMADDPE

SEQ ID NO: 40 - protein, 27 aa, Artificial Sequence
  example of serum albumin-binding agents
AEGTGDRNMCKFSWIRSPAFCARADPE

SEQ ID NO: 41 - protein, 12 aa, Artificial Sequence
  example of serum albumin-binding agents
CDRIAWYPQHAC

SEQ ID NO: 42 - protein, 12 aa, Artificial Sequence
  example of serum albumin-binding agents
CDRIAWYPQALC

SEQ ID NO: 43 - protein, 12 aa, Artificial Sequence
  example of serum albumin-binding agents
CDRIAWYPAHLC

SEQ ID NO: 44 - protein, 12 aa, Artificial Sequence
  example of serum albumin-binding agents
CDRIAWYAQHLC

SEQ ID NO: 45 - protein, 12 aa, Artificial Sequence
  example of serum albumin-binding agents
CDRIAWAPQHLC

SEQ ID NO: 46 - protein, 12 aa, Artificial Sequence
  example of serum albumin-binding agents
CDRIAAYPQHLC

SEQ ID NO: 47 - protein, 12 aa, Artificial Sequence
  example of serum albumin-binding agents
CDRAAWYPQHLC

SEQ ID NO: 48 - protein, 12 aa, Artificial Sequence
  example of serum albumin-binding agents
CDAIAWYPQHLC

SEQ ID NO: 49 - protein, 12 aa, Artificial Sequence
  example of serum albumin-binding agents
CARIAWYPQHLC

SEQ ID NO: 50 - protein, 18 aa, Artificial Sequence
  example of serum albumin-binding agents
FWFCDRIAWYPQHLCEFA

SEQ ID NO: 51 - protein, 18 aa, Artificial Sequence
  example of serum albumin-binding agents
FWFCDRIAWYPQHLCEAL

SEQ ID NO: 52 - protein, 18 aa, Artificial Sequence
  example of serum albumin-binding agents
FWFCDRIAWYPQHLCAFL

SEQ ID NO: 53 - protein, 18 aa, Artificial Sequence
  example of serum albumin-binding agents
FWFCDRIAWYPQHACEFL

SEQ ID NO: 54 - protein, 18 aa, Artificial Sequence
  example of serum albumin-binding agents
FWFCDRIAWYPQALCEFL

SEQ ID NO: 55 - protein, 18 aa, Artificial Sequence
  example of serum albumin-binding agents
FWFCDRIAWYPAHLCEFL

SEQ ID NO: 56 - protein, 18 aa, Artificial Sequence
  example of serum albumin-binding agents
FWFCDRIAWYAQHLCEFL

SEQ ID NO: 57 - protein, 18 aa, Artificial Sequence
  example of serum albumin-binding agents
FWFCDRIAWAPQHLCEFL

SEQ ID NO: 58 - protein, 18 aa, Artificial Sequence
  example of serum albumin-binding agents
FWFCDRIAAYPQHLCEFL

SEQ ID NO: 59 - protein, 18 aa, Artificial Sequence
  example of serum albumin-binding agents
FWFCDRAAWYPQHLCEFL

SEQ ID NO: 60 - protein, 18 aa, Artificial Sequence
  example of serum albumin-binding agents
FWFCDAIAWYPQHLCEFL

SEQ ID NO: 61 - protein, 18 aa, Artificial Sequence
  example of serum albumin-binding agents
FWFCARIAWYPQHLCEFL

SEQ ID NO: 62 - protein, 18 aa, Artificial Sequence
  example of serum albumin-binding agents
FWACDRIAWYPQHLCEFL

SEQ ID NO: 63 - protein, 18 aa, Artificial Sequence
  example of serum albumin-binding agents
FAFCDRIAWYPQHLCEFL

SEQ ID NO: 64 - protein, 18 aa, Artificial Sequence
  example of serum albumin-binding agents
AWFCDRIAWYPQHLCEFL

SEQ ID NO: 65 - protein, 27 aa, Artificial Sequence
  example of serum albumin-binding agents
AEGTGDFWFCDRIAWYPQHLCEFLAPE

SEQ ID NO: 66 - protein, 27 aa, Artificial Sequence
  example of serum albumin-binding agents
AEGTGDFWFCDRIAWYPQHLCEFADPE

SEQ ID NO: 67 - protein, 27 aa, Artificial Sequence
  example of serum albumin-binding agents
AEGTGDFWFCDRIAWYPQHLCEALDPE

SEQ ID NO: 68 - protein, 27 aa, Artificial Sequence
  example of serum albumin-binding agents
AEGTGDFWFCDRIAWYPQHLCAFLDPE

SEQ ID NO: 69 - protein, 27 aa, Artificial Sequence
  example of serum albumin-binding agents
AEGTGDFWFCDRIAWYPQHACEFLDPE

SEQ ID NO: 70 - protein, 27 aa, Artificial Sequence
  example of serum albumin-binding agents
AEGTGDFWFCDRIAWYPQALCEFLDPE

SEQ ID NO: 71 - protein, 27 aa, Artificial Sequence
  example of serum albumin-binding agents
AEGTGDFWFCDRIAWYPAHLCEFLDPE

SEQ ID NO: 72 - protein, 27 aa, Artificial Sequence
  example of serum albumin-binding agents
AEGTGDFWFCDRIAWYAQHLCEFLDPE

SEQ ID NO: 73 - protein, 27 aa, Artificial Sequence
  example of serum albumin-binding agents
AEGTGDFWFCDRIAWAPQHLCEFLDPE

SEQ ID NO: 74 - protein, 27 aa, Artificial Sequence
  example of serum albumin-binding agents
AEGTGDFWFCDRIAAYPQHLCEFLDPE

SEQ ID NO: 75 - protein, 27 aa, Artificial Sequence
  example of serum albumin-binding agents
AEGTGDFWFCDRAAWYPQHLCEFLDPE

SEQ ID NO: 76 - protein, 27 aa, Artificial Sequence
  example of serum albumin-binding agents
AEGTGDFWFCDAIAWYPQHLCEFLDPE

SEQ ID NO: 77 - protein, 27 aa, Artificial Sequence
  example of serum albumin-binding agents
AEGTGDFWFCARIAWYPQHLCEFLDPE

SEQ ID NO: 78 - protein, 27 aa, Artificial Sequence
  example of serum albumin-binding agents
AEGTGDFWACDRIAWYPQHLCEFLDPE

SEQ ID NO: 79 - protein, 27 aa, Artificial Sequence
  example of serum albumin-binding agents
AEGTGDFAFCDRIAWYPQHLCEFLDPE

SEQ ID NO: 80 - protein, 27 aa, Artificial Sequence
  example of serum albumin-binding agents
AEGTGDAWFCDRIAWYPQHLCEFLDPE

SEQ ID NO: 81 - protein, 27 aa, Artificial Sequence
  example of serum albumin-binding agents
AEGTGAFWFCDRIAWYPQHLCEFLDPE

SEQ ID NO: 82 - protein, 31 aa, Artificial Sequence
  example of serum albumin-binding agents
AEGTGDFWFCDRIAWYPQHLCEFLAPEGGGK

SEQ ID NO: 83 - protein, 31 aa, Artificial Sequence
  example of serum albumin-binding agents
AEGTGDFWFCDRIAWYPQHLCEFADPEGGGK

SEQ ID NO: 84 - protein, 31 aa, Artificial Sequence
  example of serum albumin-binding agents
AEGTGDFWFCDRIAWYPQHLCEALDPEGGGK

SEQ ID NO: 85 - protein, 31 aa, Artificial Sequence
  example of serum albumin-binding agents
AEGTGDFWFCDRIAWYPQHLCAFLDPEGGGK

SEQ ID NO: 86 - protein, 31 aa, Artificial Sequence
  example of serum albumin-binding agents
AEGTGDFWFCDRIAWYPQHACEFLDPEGGGK

SEQ ID NO: 87 - protein, 31 aa, Artificial Sequence
  example of serum albumin-binding agents
AEGTGDFWFCDRIAWYPQALCEFLDPEGGGK

SEQ ID NO: 88 - protein, 31 aa, Artificial Sequence
  example of serum albumin-binding agents
AEGTGDFWFCDRIAWYPAHLCEFLDPEGGGK

SEQ ID NO: 89 - protein, 31 aa, Artificial Sequence
  example of serum albumin-binding agents
AEGTGDFWFCDRIAWYAQHLCEFLDPEGGGK

SEQ ID NO: 90 - protein, 31 aa, Artificial Sequence
  example of serum albumin-binding agents
AEGTGDFWFCDRIAWAPQHLCEFLDPEGGGK

SEQ ID NO: 91 - protein, 31 aa, Artificial Sequence
  example of serum albumin-binding agents
AEGTGDFWFCDRIAAYPQHLCEFLDPEGGGK

SEQ ID NO: 92 - protein, 31 aa, Artificial Sequence
  example of serum albumin-binding agents
AEGTGDFWFCDRAAWYPQHLCEFLDPEGGGK

SEQ ID NO: 93 - protein, 31 aa, Artificial Sequence
  example of serum albumin-binding agents
AEGTGDFWFCDAIAWYPQHLCEFLDPEGGGK

SEQ ID NO: 94 - protein, 31 aa, Artificial Sequence
  example of serum albumin-binding agents
AEGTGDFWFCARIAWYPQHLCEFLDPEGGGK

SEQ ID NO: 95 - protein, 31 aa, Artificial Sequence
  example of serum albumin-binding agents
AEGTGDFWACDRIAWYPQHLCEFLDPEGGGK

SEQ ID NO: 96 - protein, 31 aa, Artificial Sequence
  example of serum albumin-binding agents
AEGTGDFAFCDRIAWYPQHLCEFLDPEGGGK

SEQ ID NO: 97 - protein, 31 aa, Artificial Sequence
  example of serum albumin-binding agents
AEGTGDAWFCDRIAWYPQHLCEFLDPEGGGK

SEQ ID NO: 98 - protein, 31 aa, Artificial Sequence
  example of serum albumin-binding agents
AEGTGAFWFCDRIAWYPQHLCEFLDPEGGGK

SEQ ID NO: 99 - protein, 14 aa, Artificial Sequence
  example of serum albumin-binding agents
XXXCXXXXXXCXXX

SEQ ID NO: 100 - protein, 18 aa, Artificial Sequence
  example of serum albumin-binding agents
AGXXXCXXXXXXCXXXGT

SEQ ID NO: 101 - protein, 10 aa, Artificial Sequence
  example of serum albumin-binding agents
CXXXXXXXXC

SEQ ID NO: 102 - protein, 16 aa, Artificial Sequence
  example of serum albumin-binding agents
XXXCXXXXXXXXCXXX

SEQ ID NO: 103 - protein, 20 aa, Artificial Sequence
  example of serum albumin-binding agents
GSXXXCXXXXXXXXCXXXAP

SEQ ID NO: 104 - protein, 20 aa, Artificial Sequence
  example of serum albumin-binding agents
PTVVQPKFHAFTHEDLLWIF

SEQ ID NO: 105 - protein, 20 aa, Artificial Sequence
  example of serum albumin-binding agents
LKSQMVHALPAASLHDQHEL

SEQ ID NO: 106 - protein, 20 aa, Artificial Sequence
  example of serum albumin-binding agents
SQVQGTPDLQFTVRDFIYMF

SEQ ID NO: 107 - protein, 12 aa, Artificial Sequence
  example of serum albumin-binding agents
CQTTWPFTMMQC

SEQ ID NO: 108 - protein, 12 aa, Artificial Sequence
  example of serum albumin-binding agents
CVTMWPFEQIFC

SEQ ID NO: 109 - protein, 12 aa, Artificial Sequence
  example of serum albumin-binding agents
CFTYYPFTTFSC

SEQ ID NO: 110 - protein, 12 aa, Artificial Sequence
  example of serum albumin-binding agents
CWTKFPFDLVWC

SEQ ID NO: 111 - protein, 12 aa, Artificial Sequence
  example of serum albumin-binding agents
CVSYWPHFVPVC

SEQ ID NO: 112 - protein, 12 aa, Artificial Sequence
  example of serum albumin-binding agents
CYISFPFDQMYC

SEQ ID NO: 113 - protein, 12 aa, Artificial Sequence
  example of serum albumin-binding agents
CSVQYPFEVVVC

SEQ ID NO: 114 - protein, 12 aa, Artificial Sequence
  example of serum albumin-binding agents
CWTQYPFDHSTC

SEQ ID NO: 115 - protein, 12 aa, Artificial Sequence
  example of serum albumin-binding agents
CITWPFKRPWPC

SEQ ID NO: 116 - protein, 12 aa, Artificial Sequence
  example of serum albumin-binding agents
CISWPFEMPFHC

SEQ ID NO: 117 - protein, 12 aa, Artificial Sequence
  example of serum albumin-binding agents
CITWPFKRPWPC

SEQ ID NO: 118 - protein, 12 aa, Artificial Sequence
  example of serum albumin-binding agents
CITYPFHEMFPC

SEQ ID NO: 119 - protein, 12 aa, Artificial Sequence
  example of serum albumin-binding agents
CITWPFQTSYPC

SEQ ID NO: 120 - protein, 12 aa, Artificial Sequence
  example of serum albumin-binding agents
CKFSWIRSPAFC

SEQ ID NO: 121 - protein, 12 aa, Artificial Sequence
  example of serum albumin-binding agents
CWIVDEDGTKWC

SEQ ID NO: 122 - protein, 12 aa, Artificial Sequence
  example of serum albumin-binding agents
CDSAYWQEIPAC

SEQ ID NO: 123 - protein, 8 aa, Artificial Sequence
  example of serum albumin-binding agents
CLWDPMLC

SEQ ID NO: 124 - protein, 12 aa, Artificial Sequence
  example of serum albumin-binding agents
CEHPYWTEVDKC

SEQ ID NO: 125 - protein, 12 aa, Artificial Sequence
  example of serum albumin-binding agents
CDTPYWRDLWQC

SEQ ID NO: 126 - protein, 12 aa, Artificial Sequence
  example of serum albumin-binding agents
CQLPYMSTPEFC

SEQ ID NO: 127 - protein, 12 aa, Artificial Sequence
  example of serum albumin-binding agents
CGRGFDKESIYC

SEQ ID NO: 128 - protein, 12 aa, Artificial Sequence
  example of serum albumin-binding agents
CVTYIGTWETVC

SEQ ID NO: 129 - protein, 12 aa, Artificial Sequence
  example of serum albumin-binding agents
CTDTNWSWMFDC

SEQ ID NO: 130 - protein, 12 aa, Artificial Sequence
  example of serum albumin-binding agents
CTLEIGTWFVFC

SEQ ID NO: 131 - protein, 12 aa, Artificial Sequence
  example of serum albumin-binding agents
CKIALFQHFEVC

SEQ ID NO: 132 - protein, 12 aa, Artificial Sequence
  example of serum albumin-binding agents
CIKLYGLGHMYC

SEQ ID NO: 133 - protein, 12 aa, Artificial Sequence
  example of serum albumin-binding agents
CEMQSIIPWWEC

SEQ ID NO: 134 - protein, 12 aa, Artificial Sequence
  example of serum albumin-binding agents
CVEKYYWDVLIC

SEQ ID NO: 135 - protein, 11 aa, Artificial Sequence
  example of serum albumin-binding agents
CPHGRYSMFPC

SEQ ID NO: 136 - protein, 12 aa, Artificial Sequence
  example of serum albumin-binding agents
CNVRWTDTPYWC

SEQ ID NO: 137 - protein, 12 aa, Artificial Sequence
  example of serum albumin-binding agents
CTYDPIADLLFC

SEQ ID NO: 138 - protein, 10 aa, Artificial Sequence
  example of serum albumin-binding agents
CMDWPNHRDC

SEQ ID NO: 139 - protein, 10 aa, Artificial Sequence
  example of serum albumin-binding agents
CFPIHLTMFC

SEQ ID NO: 140 - protein, 10 aa, Artificial Sequence
  example of serum albumin-binding agents
CQTSFTNYWC

SEQ ID NO: 141 - protein, 9 aa, Artificial Sequence
  example of serum albumin-binding agents
CMEFGPDDC

SEQ ID NO: 142 - protein, 8 aa, Artificial Sequence
  example of serum albumin-binding agents
CSWDPIFC

SEQ ID NO: 143 - protein, 8 aa, Artificial Sequence
  example of serum albumin-binding agents
CAWDPLVC

SEQ ID NO: 144 - protein, 8 aa, Artificial Sequence
  example of serum albumin-binding agents
CHIYDWFC

SEQ ID NO: 145 - protein, 8 aa, Artificial Sequence
  example of serum albumin-binding agents
CLWDPMIC

SEQ ID NO: 146 - protein, 8 aa, Artificial Sequence
  example of serum albumin-binding agents
CSPPGKTC

SEQ ID NO: 147 - protein, 8 aa, Artificial Sequence
  example of serum albumin-binding agents
CTFWQYWC

SEQ ID NO: 148 - protein, 8 aa, Artificial Sequence
  example of serum albumin-binding agents
CMFELPFC

SEQ ID NO: 149 - protein, 8 aa, Artificial Sequence
  example of serum albumin-binding agents
CFSKPDQC

SEQ ID NO: 150 - protein, 8 aa, Artificial Sequence
  example of serum albumin-binding agents
CFYQWWGC

SEQ ID NO: 151 - protein, 8 aa, Artificial Sequence
  example of serum albumin-binding agents
CTWDPIFC

SEQ ID NO: 152 - protein, 6 aa, Artificial Sequence
  example of serum albumin-binding agents
CWLYDC

SEQ ID NO: 153 - protein, 6 aa, Artificial Sequence
  example of serum albumin-binding agents
CDKYGC

SEQ ID NO: 154 - protein, 6 aa, Artificial Sequence
  example of serum albumin-binding agents
CSKDTC

SEQ ID NO: 155 - protein, 17 aa, Artificial Sequence
  example of serum albumin-binding agents
LRDCQTTWPFMMQCPNN

SEQ ID NO: 156 - protein, 18 aa, Artificial Sequence
  example of serum albumin-binding agents
NRECVTMWPFEQIFCPWP

SEQ ID NO: 157 - protein, 18 aa, Artificial Sequence
  example of serum albumin-binding agents
LRSCFTYYPFTTFSCSPA

SEQ ID NO: 158 - protein, 18 aa, Artificial Sequence
  example of serum albumin-binding agents
LSHCWTKFPFDLVWCDSP

SEQ ID NO: 159 - protein, 18 aa, Artificial Sequence
  example of serum albumin-binding agents
LRMCVSYWPHFVPVCENP

SEQ ID NO: 160 - protein, 18 aa, Artificial Sequence
  example of serum albumin-binding agents
LRDCYISFPFDQMYCSHF

SEQ ID NO: 161 - protein, 18 aa, Artificial Sequence
  example of serum albumin-binding agents
FRHCSVQYPFEVVVCPAN

SEQ ID NO: 162 - protein, 18 aa, Artificial Sequence
  example of serum albumin-binding agents
LRNCWTQYPFDHSTCSPN

SEQ ID NO: 163 - protein, 17 aa, Artificial Sequence
  example of serum albumin-binding agents
DSMCITWPFKRPWPCAN

SEQ ID NO: 164 - protein, 18 aa, Artificial Sequence
  example of serum albumin-binding agents
AFMCISWPFEMPFHCSPD

SEQ ID NO: 165 - protein, 18 aa, Artificial Sequence
  example of serum albumin-binding agents
DSMCITWPFKRPWPCANP

SEQ ID NO: 166 - protein, 18 aa, Artificial Sequence
  example of serum albumin-binding agents
WDLCITYPFHEMFPCEDG

SEQ ID NO: 167 - protein, 18 aa, Artificial Sequence
  example of serum albumin-binding agents
GGECITWPFQTSYPCTNG

SEQ ID NO: 168 - protein, 18 aa, Artificial Sequence
  example of serum albumin-binding agents
RNMCKFSWIRSPAFCARA

SEQ ID NO: 169 - protein, 17 aa, Artificial Sequence
  example of serum albumin-binding agents
FSLCWIVDEDGTKWCLP

SEQ ID NO: 170 - protein, 18 aa, Artificial Sequence
  example of serum albumin-binding agents
RWFCDSAYWQEIPACARD

SEQ ID NO: 171 - protein, 14 aa, Artificial Sequence
  example of serum albumin-binding agents
RWYCLWDPMLCMSD

SEQ ID NO: 172 - protein, 18 aa, Artificial Sequence
  example of serum albumin-binding agents
AWYCEHPYWTEVDKCHSS

SEQ ID NO: 173 - protein, 18 aa, Artificial Sequence
  example of serum albumin-binding agents
SDFCDTPYWRDLWQCNSP

SEQ ID NO: 174 - protein, 18 aa, Artificial Sequence
  example of serum albumin-binding agents
LPWCQLPYMSTPEFCIRP

SEQ ID NO: 175 - protein, 18 aa, Artificial Sequence
  example of serum albumin-binding agents
YHVCGRGFDKESIYCKFL

SEQ ID NO: 176 - protein, 17 aa, Artificial Sequence
  example of serum albumin-binding agents
SFCVTYIGTWETVCKRS

SEQ ID NO: 177 - protein, 18 aa, Artificial Sequence
  example of serum albumin-binding agent
NDGCTDTNWSWMFDCPPL

SEQ ID NO: 178 - protein, 18 aa, Artificial Sequence
  example of serum albumin-binding agent
WRDCTLEIGTWFVFCKGS

SEQ ID NO: 179 - protein, 18 aa, Artificial Sequence
  example of serum albumin-binding agent
SPYCKIALFQHFEVCAAD

SEQ ID NO: 180 - protein, 18 aa, Artificial Sequence
  example of serum albumin-binding agent
RHWCIKLYGLGHMYCNRS

SEQ ID NO: 181 - protein, 18 aa, Artificial Sequence
  serum albumin-binding agent
DHACEMQSIIPWWECYPH

SEQ ID NO: 182 - protein, 18 aa, Artificial Sequence
  example of serum albumin-binding agent
PRSCVEKYYWDVLICGFF

SEQ ID NO: 183 - protein, 17 aa, Artificial Sequence
  example of serum albumin-binding agent
FHTCPHGRYSMFPCDYW

SEQ ID NO: 184 - protein, 18 aa, Artificial Sequence
  example of serum albumin-binding agent
HGWCNVRWTDTPYWCAFS

SEQ ID NO: 185 - protein, 18 aa, Artificial Sequence
  example of serum albumin-binding agent
YRVCTYDPIADLLFCPFN

SEQ ID NO: 186 - protein, 16 aa, Artificial Sequence
  example of serum albumin-binding agent
RSFCMDWPNHRDCDYS

SEQ ID NO: 187 - protein, 16 aa, Artificial Sequence
  example of serum albumin-binding agent
FWDCFPIHLTMFCDRF

SEQ ID NO: 188 - protein, 16 aa, Artificial Sequence
  example of serum albumin-binding agent
YLYCQTSFTNYWCAFH

SEQ ID NO: 189 - protein, 15 aa, Artificial Sequence
  example of serum albumin-binding agent
GLYCMEFGPDDCAWH

SEQ ID NO: 190 - protein, 14 aa, Artificial Sequence
  example of serum albumin-binding agent
KNFCSWDPIFCGIH

SEQ ID NO: 191 - protein, 14 aa, Artificial Sequence
  example of serum albumin-binding agent
KWYCAWDPLVCEIF

SEQ ID NO: 192 - protein, 14 aa, Artificial Sequence
  example of serum albumin-binding agent
WTTCHIYDWFCSSS

SEQ ID NO: 193 - protein, 14 aa, Artificial Sequence
  example of serum albumin-binding agent
QWYCLWDPMICGLI

SEQ ID NO: 194 - protein, 14 aa, Artificial Sequence
  example of serum albumin-binding agent
QTNCSPPGKTCDKN

SEQ ID NO: 195 - protein, 13 aa, Artificial Sequence
  example of serum albumin-binding agent
AICTFWQYWCLEP

SEQ ID NO: 196 - protein, 14 aa, Artificial Sequence
  example of serum albumin-binding agent
FEWCMFELPFCSWP

SEQ ID NO: 197 - protein, 14 aa, Artificial Sequence
  example of serum albumin-binding agent
QEGCFSKPDQCKVM

SEQ ID NO: 198 - protein, 14 aa, Artificial Sequence
  example of serum albumin-binding agent
LEYCFYQWWGCPHA

SEQ ID NO: 199 - protein, 14 aa, Artificial Sequence
  example of serum albumin-binding agent
YQFCTWDPIFCGWH

SEQ ID NO: 200 - protein, 12 aa, Artificial Sequence
  example of serum albumin-binding agent
LWDCWLYDCEGN

SEQ ID NO: 201 - protein, 12 aa, Artificial Sequence
  example of serum albumin-binding agent
VHSCDKYGCVNA

SEQ ID NO: 202 - protein, 12 aa, Artificial Sequence
  example of serum albumin-binding agent
FEHCSKDTCSGN

SEQ ID NO: 203 - protein, 12 aa, Artificial Sequence
  example of serum albumin-binding agent
VAWCTIFLCLDV

SEQ ID NO: 204 - protein, 12 aa, Artificial Sequence
  example of serum albumin-binding agent
FKICDQWFCLMP

SEQ ID NO: 205 - protein, 12 aa, Artificial Sequence
  example of serum albumin-binding agent
HVGCNNALCMQY

SEQ ID NO: 206 - protein, 12 aa, Artificial Sequence
  example of serum albumin-binding agent
WKVCDHFFCLSP

SEQ ID NO: 207 - protein, 12 aa, Artificial Sequence
  example of serum albumin-binding agent
NHGCWHFSCIWD

SEQ ID NO: 208 - protein, 16 aa, Artificial Sequence
  example of serum albumin-binding agent
FRNCEPWMLRFGCNPR

SEQ ID NO: 209 - protein, 18 aa, Artificial Sequence
  example of serum albumin-binding agent
ADFCEGKDMIDWVYCRLY

SEQ ID NO: 210 - protein, 19 aa, Artificial Sequence
  example of serum albumin-binding agent
FWFCDRIAWYPQHLCEFLD

SEQ ID NO: 211 - protein, 18 aa, Artificial Sequence
  example of serum albumin-binding agent
DWDCVTRWANRDQQCWGP

SEQ ID NO: 212 - protein, 18 aa, Artificial Sequence
  example of serum albumin-binding agent
DWDCVTRWANRDQQCWAL

SEQ ID NO: 213 - protein, 18 aa, Artificial Sequence
  example of serum albumin-binding agent
DWDCVTDWANRHQHCWAL

SEQ ID NO: 214 - protein, 18 aa, Artificial Sequence
  example of serum albumin-binding agent
DWQCVKDWANRRRGCMAD

SEQ ID NO: 215 - protein, 20 aa, Artificial Sequence
  example of serum albumin-binding agent
RNMCKFSWIRSPAFCARADP

SEQ ID NO: 216 - protein, 26 aa, Artificial Sequence
  serum albumin-binding agent
GDLRDCQTTWPFTMMQCPNNDPGGGK

SEQ ID NO: 217 - protein, 26 aa, Artificial Sequence
  serum albumin-binding agent
GDNRECVTMWPFEQIFCPWPDPGGGK

SEQ ID NO: 218 - protein, 26 aa, Artificial Sequence
  serum albumin-binding agent
GDLRSCFTYYPFTTFSCSPADPGGGK

SEQ ID NO: 219 - protein, 25 aa, Artificial Sequence
  serum albumin-binding agent
GDDSMCITWPFKRPWPCANDPGGGK

SEQ ID NO: 220 - protein, 26 aa, Artificial Sequence
  serum albumin-binding agent
GDRNMCKFSWIRSPAFCARADPGGGK

SEQ ID NO: 221 - protein, 25 aa, Artificial Sequence
  serum albumin-binding agent
GDFSLCWIVDEDGTKWCLPDPGGGK

SEQ ID NO: 222 - protein, 26 aa, Artificial Sequence
  serum albumin-binding agent
GDRWFCDSAYWQEIPACARDDPGGGK

SEQ ID NO: 223 - protein, 26 aa, Artificial Sequence
  serum albumin-binding agent
GDSDFCDTPYWRDLWQCNSPDPGGGK

SEQ ID NO: 224 - protein, 25 aa, Artificial Sequence
  serum albumin-binding agent
GDSFCVTYIGTWETVCKRSDPGGGK

SEQ ID NO: 225 - protein, 26 aa, Artificial Sequence
  serum albumin-binding agent
GDNDGCTDTNWSWMFDCPPLDPGGGK

SEQ ID NO: 226 - protein, 26 aa, Artificial Sequence
  serum albumin-binding agent
GDSPYCKIALFQHFEVCAADDPGGGK

SEQ ID NO: 227 - protein, 26 aa, Artificial Sequence
  serum albumin-binding agent
GDPRSCVEKYYWDVLICGFFDPGGGK

SEQ ID NO: 228 - protein, 24 aa, Artificial Sequence
  serum albumin-binding agent
GSRSFCMDWPNHRDCDYSAPGGGK

SEQ ID NO: 229 - protein, 22 aa, Artificial Sequence
  serum albumin-binding agent
AGKWYCAWDPLVCEIFGTGGGK

SEQ ID NO: 230 - protein, 22 aa, Artificial Sequence
  serum albumin-binding agent
AGWTTCHIYDWFCSSSGTGGGK

SEQ ID NO: 231 - protein, 22 aa, Artificial Sequence
  serum albumin-binding agent
AGLEYCFYQWWGCPHAGTGGGK

SEQ ID NO: 232 - protein, 22 aa, Artificial Sequence
  serum albumin-binding agent
AGYQFCTWDPIFCGWHGTGGGK

SEQ ID NO: 233 - protein, 20 aa, Artificial Sequence
  serum albumin-binding agent
GSLWDCWLYDCEGNAPGGGK

SEQ ID NO: 234 - protein, 8 aa, Artificial Sequence
  exemplary motif
XXXXXWCX

SEQ ID NO: 235 - protein, 8 aa, Artificial Sequence
  exemplary motif
XXWXXXWX

SEQ ID NO: 236 - protein, 6 aa, Artificial Sequence
  exemplary motif
XWXWWX

SEQ ID NO: 237 - protein, 9 aa, Artificial Sequence
  exemplary motif
XPXWXCXXX

SEQ ID NO: 238 - protein, 18 aa, Artificial Sequence
  immunoglobulin binding polypeptide
RRACSRDWSGALVWCAGH

SEQ ID NO: 239 - protein, 18 aa, Artificial Sequence
  immunoglobulin binding polypeptide
WGECTVTSYGELIWCGGL

SEQ ID NO: 240 - protein, 18 aa, Artificial Sequence
  immunoglobulin binding polypeptide
SSACAFDPMGAVIWCTYD

SEQ ID NO: 241 - protein, 18 aa, Artificial Sequence
  immunoglobulin binding polypeptide
LLECAYNTSGELIWCNGS

SEQ ID NO: 242 - protein, 18 aa, Artificial Sequence
  immunoglobulin binding polypeptide
PDDCSIHFSGELIWCEPL

SEQ ID NO: 243 - protein, 18 aa, Artificial Sequence
  immunoglobulin binding polypeptide
LGECTVTSYGELIWCGGL

SEQ ID NO: 244 - protein, 18 aa, Artificial Sequence
  immunoglobulin binding polypeptide
WGECTVTSYGELIWCGGH

SEQ ID NO: 245 - protein, 18 aa, Artificial Sequence
  immunoglobulin binding polypeptide
DHMCVYTTWGELIWCDDH

SEQ ID NO: 246 - protein, 18 aa, Artificial Sequence
  immunoglobulin binding polypeptide
WGECTVTSYGELIWCGGL

SEQ ID NO: 247 - protein, 18 aa, Artificial Sequence
  immunoglobulin binding polypeptide
CRACSRDWPGALVWCAGH

SEQ ID NO: 248 - protein, 18 aa, Artificial Sequence
  immunoglobulin binding polypeptide
RRACSRDWSGALVWCAGH

SEQ ID NO: 249 - protein, 18 aa, Artificial Sequence
  immunoglobulin binding polypeptide
LHACAFDPMGAVIWCTYD

SEQ ID NO: 250 - protein, 18 aa, Artificial Sequence
  immunoglobulin binding polypeptide
DHMCVYTTWGELMWCDNH

SEQ ID NO: 251 - protein, 18 aa, Artificial Sequence
  immunoglobulin binding polypeptide
PPTCTWDWQGILVWCSGH

SEQ ID NO: 252 - protein, 18 aa, Artificial Sequence
  immunoglobulin binding polypeptide
SNKCSNTWDGSLIWCSAN

SEQ ID NO: 253 - protein, 18 aa, Artificial Sequence
  immunoglobulin binding polypeptide
FPECTFDMEGFLIWCSSF

SEQ ID NO: 254 - protein, 18 aa, Artificial Sequence
  immunoglobulin binding polypeptide
HDLCAQAPFGDATWCDLR

SEQ ID NO: 255 - protein, 18 aa, Artificial Sequence
  immunoglobulin binding polypeptide
PNHCSYNLKSELIWCQDL

SEQ ID NO: 256 - protein, 18 aa, Artificial Sequence
  immunoglobulin binding polypeptide
PLDCARDIHNSLIWCSLG

SEQ ID NO: 257 - protein, 18 aa, Artificial Sequence
  immunoglobulin binding polypeptide
GSECSWTSLNELIWCAHW

SEQ ID NO: 258 - protein, 18 aa, Artificial Sequence
  immunoglobulin binding polypeptide
WPDCSFTVQRDLIWCEAL

SEQ ID NO: 259 - protein, 18 aa, Artificial Sequence
  immunoglobulin binding polypeptide
SHSCAYDYAHMLVWCTHF

SEQ ID NO: 260 - protein, 18 aa, Artificial Sequence
  immunoglobulin binding polypeptide
DHMCVYTTWGELIWCDNH

SEQ ID NO: 261 - protein, 18 aa, Artificial Sequence
  immunoglobulin binding polypeptide
RPNCTFAASGELIWCMHY

SEQ ID NO: 262 - protein, 18 aa, Artificial Sequence
  immunoglobulin binding polypeptide
WWGCQFDWRGELVWCPYL

SEQ ID NO: 263 - protein, 18 aa, Artificial Sequence
  immunoglobulin binding polypeptide
GGVCSYSGMGEIVWCRWF

SEQ ID NO: 264 - protein, 18 aa, Artificial Sequence
  immunoglobulin binding polypeptide
ALMCSHDMWGSLIWCKHF

SEQ ID NO: 265 - protein, 18 aa, Artificial Sequence
  immunoglobulin binding polypeptide
WWNCHNGWTWTGGWCWWF

SEQ ID NO: 266 - protein, 18 aa, Artificial Sequence
  immunoglobulin binding polypeptide
YHVCARDSWDQLIWCEAF

SEQ ID NO: 267 - protein, 15 aa, Artificial Sequence
  immunoglobulin binding polypeptide
NYWCNFWQLPTCDNL

SEQ ID NO: 268 - protein, 16 aa, Artificial Sequence
  immunoglobulin binding polypeptide
YWYCKWFSESASCSSR

SEQ ID NO: 269 - protein, 16 aa, Artificial Sequence
  immunoglobulin binding polypeptide
YWYCKWFEDKHPCDSS

SEQ ID NO: 270 - protein, 16 aa, Artificial Sequence
  immunoglobulin binding polypeptide
YWYCSWFPDRPDCPLY

SEQ ID NO: 271 - protein, 16 aa, Artificial Sequence
  immunoglobulin binding polypeptide
NYWCNVWLLGDVCRSH

SEQ ID NO: 272 - protein, 18 aa, Artificial Sequence
  immunoglobulin binding polypeptide
LYWCHVWFGQHAWQCKYP

SEQ ID NO: 273 - protein, 14 aa, Artificial Sequence
  immunoglobulin binding polypeptide
YWKCKWMPWMCGFD

SEQ ID NO: 274 - protein, 18 aa, Artificial Sequence
  immunoglobulin binding polypeptide
DDHCYWFREWFNSECPHG

SEQ ID NO: 275 - protein, 15 aa, Artificial Sequence
  immunoglobulin binding polypeptide
NYWCNIWGLHGCNSH

SEQ ID NO: 276 - protein, 16 aa, Artificial Sequence
  immunoglobulin binding polypeptide
YWFCQWFSQNHTCFRD

SEQ ID NO: 277 - protein, 16 aa, Artificial Sequence
  immunoglobulin binding polypeptide
HYWCDIWFGAPACQFR

SEQ ID NO: 278 - protein, 17 aa, Artificial Sequence
  immunoglobulin binding polypeptide
SGDCGFWPRIWGLCMDN

SEQ ID NO: 279 - protein, 16 aa, Artificial Sequence
  immunoglobulin binding polypeptide
FWYCKWFYEDAQCSHD

SEQ ID NO: 280 - protein, 13 aa, Artificial Sequence
  immunoglobulin binding polypeptide
YYWCNYWGLCPDQ

SEQ ID NO: 281 - protein, 13 aa, Artificial Sequence
  immunoglobulin binding polypeptide
SYWCKIWDVCPQS

SEQ ID NO: 282 - protein, 13 aa, Artificial Sequence
  immunoglobulin binding polypeptide
KYWCNLWGVCPAN

SEQ ID NO: 283 - protein, 13 aa, Artificial Sequence
  immunoglobulin binding polypeptide
QYWCYQWGLCGAN

SEQ ID NO: 284 - protein, 13 aa, Artificial Sequence
  immunoglobulin binding polypeptide
KYWCQQWGVCNGS

SEQ ID NO: 285 - protein, 13 aa, Artificial Sequence
  immunoglobulin binding polypeptide
KYWCVQWGVCPES

SEQ ID NO: 286 - protein, 13 aa, Artificial Sequence
  immunoglobulin binding polypeptide
KYWCMQWGLCGWE

SEQ ID NO: 287 - protein, 13 aa, Artificial Sequence
  immunoglobulin binding polypeptide
HFWCEVWGLCPSI

SEQ ID NO: 288 - protein, 13 aa, Artificial Sequence
  immunoglobulin binding polypeptide
QYWCTKWGLCTNV

SEQ ID NO: 289 - protein, 13 aa, Artificial Sequence
  immunoglobulin binding polypeptide
AYWCKVWGLCQGE

SEQ ID NO: 290 - protein, 13 aa, Artificial Sequence
  immunoglobulin binding polypeptide
KYWCNLWGVCPAN

SEQ ID NO: 291 - protein, 13 aa, Artificial Sequence
  immunoglobulin binding polypeptide
QYWCNVWGVCLPS

SEQ ID NO: 292 - protein, 13 aa, Artificial Sequence
  immunoglobulin binding polypeptide
HYWCQQWGICERP

SEQ ID NO: 293 - protein, 13 aa, Artificial Sequence
  immunoglobulin binding polypeptide
RYWCNIWDVCPEQ

SEQ ID NO: 294 - protein, 13 aa, Artificial Sequence
  immunoglobulin binding polypeptide
QYWCTHWGLCGKY

SEQ ID NO: 295 - protein, 13 aa, Artificial Sequence
  immunoglobulin binding polypeptide
TYWCTKWGLCPHN

SEQ ID NO: 296 - protein, 13 aa, Artificial Sequence
  immunoglobulin binding polypeptide
FYWCGQWGLCAPP

SEQ ID NO: 297 - protein, 13 aa, Artificial Sequence
  immunoglobulin binding polypeptide
GYWCNVWGLCSTE

SEQ ID NO: 298 - protein, 13 aa, Artificial Sequence
  immunoglobulin binding polypeptide
RYWCGVWGVCEID

SEQ ID NO: 299 - protein, 13 aa, Artificial Sequence
  immunoglobulin binding polypeptide
KFWCTIWGVCHMP

SEQ ID NO: 300 - protein, 13 aa, Artificial Sequence
  immunoglobulin binding polypeptide
HYWCQQWGICERP

SEQ ID NO: 301 - protein, 13 aa, Artificial Sequence
  immunoglobulin binding polypeptide
RYWCNIWDVCPEQ

SEQ ID NO: 302 - protein, 13 aa, Artificial Sequence
  immunoglobulin binding polypeptide
FYWCSQWGLCKYD

SEQ ID NO: 303 - protein, 13 aa, Artificial Sequence
  immunoglobulin binding polypeptide
HYWCEKWGLCLMS

SEQ ID NO: 304 - protein, 13 aa, Artificial Sequence
  immunoglobulin binding polypeptide
HYWCQKWGVCPTD

SEQ ID NO: 305 - protein, 13 aa, Artificial Sequence
  immunoglobulin binding polypeptide
HYWCSLWGVCDIN

SEQ ID NO: 306 - protein, 12 aa, Artificial Sequence
  immunoglobulin binding polypeptide
RFWCSAWGVCPA

SEQ ID NO: 307 - protein, 13 aa, Artificial Sequence
  immunoglobulin binding polypeptide
SYWCKIWDVCPQS

SEQ ID NO: 308 - protein, 13 aa, Artificial Sequence
  immunoglobulin binding polypeptide
QYWCSIWKVCPGR

SEQ ID NO: 309 - protein, 13 aa, Artificial Sequence
  immunoglobulin binding polypeptide
YWYCEWFGACIND

SEQ ID NO: 310 - protein, 14 aa, Artificial Sequence
  immunoglobulin binding polypeptide
EYWCKYWGLECVHR

SEQ ID NO: 311 - protein, 14 aa, Artificial Sequence
  immunoglobulin binding polypeptide
KYWCTQWGLKCDKQ

SEQ ID NO: 312 - protein, 13 aa, Artificial Sequence
  immunoglobulin binding polypeptide
KYWCSFWGLQCKT

SEQ ID NO: 313 - protein, 14 aa, Artificial Sequence
  immunoglobulin binding polypeptide
RYWCNFWGVNCDAN

SEQ ID NO: 314 - protein, 14 aa, Artificial Sequence
  immunoglobulin binding polypeptide
NYWCTHWGVMCLDH

SEQ ID NO: 315 - protein, 14 aa, Artificial Sequence
  immunoglobulin binding polypeptide
YWFCKWFPSQCQFM

SEQ ID NO: 316 - protein, 14 aa, Artificial Sequence
  immunoglobulin binding polypeptide
AYWCKQWGLKCQLG

SEQ ID NO: 317 - protein, 14 aa, Artificial Sequence
  immunoglobulin binding polypeptide
KYWCKFWGLECKVG

SEQ ID NO: 318 - protein, 14 aa, Artificial Sequence
  immunoglobulin binding polypeptide
NYWCTEWGLNCNNK

SEQ ID NO: 319 - protein, 14 aa, Artificial Sequence
  immunoglobulin binding polypeptide
SYWCEKWGLTCETH

SEQ ID NO: 320 - protein, 14 aa, Artificial Sequence
  immunoglobulin binding polypeptide
EYWCRIWGLQCNMV

SEQ ID NO: 321 - protein, 14 aa, Artificial Sequence
  immunoglobulin binding polypeptide
KYWCKKWGVNCDFN

SEQ ID NO: 322 - protein, 14 aa, Artificial Sequence
  immunoglobulin binding polypeptide
KYWCSVWGVQCPHS

SEQ ID NO: 323 - protein, 14 aa, Artificial Sequence
  immunoglobulin binding polypeptide
FYWCTKWGLECIHS

SEQ ID NO: 324 - protein, 14 aa, Artificial Sequence
  immunoglobulin binding polypeptide
HYWCQQWGLMCFET

SEQ ID NO: 325 - protein, 14 aa, Artificial Sequence
  immunoglobulin binding polypeptide
KYWCKRWGLMCNGG

SEQ ID NO: 326 - protein, 14 aa, Artificial Sequence
  immunoglobulin binding polypeptide
AYWCMTWGVPCISW

SEQ ID NO: 327 - protein, 14 aa, Artificial Sequence
  immunoglobulin binding polypeptide
KYWCKKWGVNCDFN

SEQ ID NO: 328 - protein, 14 aa, Artificial Sequence
  immunoglobulin binding polypeptide
KYWCSVWGVQCPDS

SEQ ID NO: 329 - protein, 14 aa, Artificial Sequence
  immunoglobulin binding polypeptide
KYWCSVWGVQCPHS

SEQ ID NO: 330 - protein, 14 aa, Artificial Sequence
  immunoglobulin binding polypeptide
LYWCTKWGVTCQKD

SEQ ID NO: 331 - protein, 14 aa, Artificial Sequence
  immunoglobulin binding polypeptide
TYWCHKWGVKCATT

SEQ ID NO: 332 - protein, 14 aa, Artificial Sequence
  immunoglobulin binding polypeptide
TYWCTFWELPCDPA

SEQ ID NO: 333 - protein, 14 aa, Artificial Sequence
  immunoglobulin binding polypeptide
KYWCTKWQLNCEEV

SEQ ID NO: 334 - protein, 14 aa, Artificial Sequence
  immunoglobulin binding polypeptide
NYWCHFWQVPCLEQ

SEQ ID NO: 335 - protein, 14 aa, Artificial Sequence
  immunoglobulin binding polypeptide
TYWCVVWNVPCSTD

SEQ ID NO: 336 - protein, 14 aa, Artificial Sequence
  immunoglobulin binding polypeptide
NFWCHTWGLQCNDL

SEQ ID NO: 337 - protein, 14 aa, Artificial Sequence
  immunoglobulin binding polypeptide
FWYCYWFNEKCKTP

SEQ ID NO: 338 - protein, 14 aa, Artificial Sequence
  immunoglobulin binding polypeptide
GFWCTFWGVTCEAG

SEQ ID NO: 339 - protein, 14 aa, Artificial Sequence
  immunoglobulin binding polypeptide
PHNCDDHYWYCKWF

SEQ ID NO: 340 - protein, 14 aa, Artificial Sequence
  immunoglobulin binding polypeptide
EMTCSSHYWYCTWM

SEQ ID NO: 341 - protein, 14 aa, Artificial Sequence
  immunoglobulin binding polypeptide
HIDCKTNYWWCRWT

SEQ ID NO: 342 - protein, 14 aa, Artificial Sequence
  immunoglobulin binding polypeptide
EMRCGQHFWYCEWF

SEQ ID NO: 343 - protein, 15 aa, Artificial Sequence
  immunoglobulin binding polypeptide
NYWCNFWQLPTCDNL

SEQ ID NO: 344 - protein, 16 aa, Artificial Sequence
  immunoglobulin binding polypeptide
YWYCQWFQEVNKCFNS

SEQ ID NO: 345 - protein, 16 aa, Artificial Sequence
  immunoglobulin binding polypeptide
YYWCRHWFPDFDCVHS

SEQ ID NO: 346 - protein, 16 aa, Artificial Sequence
  immunoglobulin binding polypeptide
YWYCSWFPDRPDCPLY

SEQ ID NO: 347 - protein, 16 aa, Artificial Sequence
  immunoglobulin binding polypeptide
YWYCVWFDNADQCVHH

SEQ ID NO: 348 - protein, 18 aa, Artificial Sequence
  immunoglobulin binding polypeptide
AATCSTSYWYYQWFCTDS

SEQ ID NO: 349 - protein, 14 aa, Artificial Sequence
  immunoglobulin binding polypeptide
YWACVWGLKSCVDR

SEQ ID NO: 350 - protein, 13 aa, Artificial Sequence
  immunoglobulin binding polypeptide
YWRCVWFPASCPT

SEQ ID NO: 351 - protein, 18 aa, Artificial Sequence
  immunoglobulin binding polypeptide
DWQCLWWGNSFWPYCANL

SEQ ID NO: 352 - protein, 14 aa, Artificial Sequence
  immunoglobulin binding polypeptide
FWRCHWWPERCPVD

SEQ ID NO: 353 - protein, 18 aa, Artificial Sequence
  immunoglobulin binding polypeptide
NPMCWKKSWWEDAYCINH

SEQ ID NO: 354 - protein, 18 aa, Artificial Sequence
  immunoglobulin binding polypeptide
SWVCWKAKWWEDKRCAPF

SEQ ID NO: 355 - protein, 18 aa, Artificial Sequence
  immunoglobulin binding polypeptide
SRQCWKELWWTDQMCLDL

SEQ ID NO: 356 - protein, 16 aa, Artificial Sequence
  immunoglobulin binding polypeptide
SFRCQSSFPSWYCDYY

SEQ ID NO: 357 - protein, 16 aa, Artificial Sequence
  immunoglobulin binding polypeptide
SWHCQNTYPEWYCQWY

SEQ ID NO: 358 - protein, 17 aa, Artificial Sequence
  immunoglobulin binding polypeptide
GSKCKQTGFPRWWCEHY

SEQ ID NO: 359 - protein, 18 aa, Artificial Sequence
  immunoglobulin binding polypeptide
DGVCGPRGFGPAWFCMHY

SEQ ID NO: 360 - protein, 16 aa, Artificial Sequence
  immunoglobulin binding polypeptide
YSHCATHYPTWYCLHF

SEQ ID NO: 361 - protein, 16 aa, Artificial Sequence
  immunoglobulin binding polypeptide
FCNCWGSHEFTFCVDD

SEQ ID NO: 362 - protein, 18 aa, Artificial Sequence
  immunoglobulin binding polypeptide
PGWCYSDIWGFKHFCNLD

SEQ ID NO: 363 - protein, 16 aa, Artificial Sequence
  immunoglobulin binding polypeptide
DSSCIKHHNKVTCFFP

SEQ ID NO: 364 - protein, 13 aa, Artificial Sequence
  immunoglobulin binding polypeptide
RWSCWGVWGCVWV

SEQ ID NO: 365 - protein, 14 aa, Artificial Sequence
  immunoglobulin binding polypeptide
PVDCKHHFWWCYWN

SEQ ID NO: 366 - protein, 17 aa, Artificial Sequence
  immunoglobulin binding polypeptide
SWNCAFHHNEMVWCDDG

SEQ ID NO: 367 - protein, 15 aa, Artificial Sequence
  immunoglobulin binding polypeptide
YWYCWFPDRPECPLY

SEQ ID NO: 368 - protein, 29 aa, Artificial Sequence
  immunoglobulin binding polypeptide
GDDHMCVYTTWGELIWCDNHEPGPEGGGK

SEQ ID NO: 369 - protein, 29 aa, Artificial Sequence
  immunoglobulin binding polypeptide
GDRRACSRDWSGALVWCAGHEPGPEGGGK

SEQ ID NO: 370 - protein, 25 aa, Artificial Sequence
  immunoglobulin binding polypeptide
AGKYWCSFWGLQCKTGTPGPEGGGK

SEQ ID NO: 371 - protein, 23 aa, Artificial Sequence
  immunoglobulin binding polypeptide
AGSYWCKIWDVCPQSPGPEGGGK

SEQ ID NO: 372 - protein, 23 aa, Artificial Sequence
  immunoglobulin binding polypeptide
AGKYWCNLWGVCPANPGPEGGGK

SEQ ID NO: 373 - protein, 24 aa, Artificial Sequence
  immunoglobulin binding polypeptide
AGTYWCTFWELPCDPAPGPEGGGK

SEQ ID NO: 374 - protein, 24 aa, Artificial Sequence
  immunoglobulin binding polypeptide
AGPHNCDDHYWYCKWFPGPEGGGK

SEQ ID NO: 375 - protein, 28 aa, Artificial Sequence
  immunoglobulin binding polypeptide
AGAATCSTSYWYYQWFCTDSPGPEGGGK

SEQ ID NO: 376 - protein, 25 aa, Artificial Sequence
  immunoglobulin binding polypeptide
AGYWYCWFPDRPECPLYPGPEGGGK

SEQ ID NO: 377 - protein, 26 aa, Artificial Sequence
  immunoglobulin binding polypeptide
AGPVDCKHHFWWCYWNGTPGPEGGGK

SEQ ID NO: 378 - protein, 29 aa, Artificial Sequence
  immunoglobulin binding polypeptide
GDDDHCYWFREWFNSECPHGEPGPEGGGK

SEQ ID NO: 379 - protein, 25 aa, Artificial Sequence
  immunoglobulin binding polypeptide
AGYYWCNYWGLCPDQGTPGPEGGGK

SEQ ID NO: 380 - protein, 30 aa, Artificial Sequence
  immunoglobulin binding polypeptide
GDSWVCWKAKWWEDKRCAPFGTPGPEGGGK

SEQ ID NO: 381 - protein, 30 aa, Artificial Sequence
  immunoglobulin binding polypeptide
GDNPMCWKKSWWEDAYCINHGTPGPEGGGK

SEQ ID NO: 382 - protein, 29 aa, Artificial Sequence
  immunoglobulin binding polypeptide
GDSWNCAFHHNEMVWCDDGGTPGPEGGGK

SEQ ID NO: 383 - protein, 29 aa, Artificial Sequence
  immunoglobulin binding polypeptide
GDWGECTVTSYGELIWCGGLEPGPEGGGK

SEQ ID NO: 384 - protein, 29 aa, Artificial Sequence
  immunoglobulin binding polypeptide
GDNPMCWRASWWEDAYCINHEPGPEGGGK

SEQ ID NO: 385 - protein, 29 aa, Artificial Sequence
  immunoglobulin binding polypeptide
GDNPMCWRAHWWEDAYCINHEPGPEGGGK

SEQ ID NO: 386 - protein, 27 aa, Artificial Sequence
  immunoglobulin binding polypeptide
GDDHMCVYTTWGELIWCDNHEPGPEGX

SEQ ID NO: 387 - protein, 24 aa, Artificial Sequence
  immunoglobulin binding polypeptide
GDDHMCVYTTWGELIWCDNHEPGX

SEQ ID NO: 388 - protein, 24 aa, Artificial Sequence
  immunoglobulin binding polypeptide
GDDHMCVYTTWGELIWCDNHEPGX

SEQ ID NO: 389 - protein, 21 aa, Artificial Sequence
  immunoglobulin binding polypeptide
GDDHMCVYTTWGELIWCDNHX

SEQ ID NO: 390 - protein, 21 aa, Artificial Sequence
  immunoglobulin binding polypeptide
GDDHMCVYTTWGELIWCDNHX

SEQ ID NO: 391 - protein, 21 aa, Artificial Sequence
  immunoglobulin binding polypeptide
GDDHMCVYTTWGELIWCDNHX

SEQ ID NO: 392 - protein, 27 aa, Artificial Sequence
  immunoglobulin binding polypeptide
DHMCVYTTWGELIWCDNHEPGPEGGGK

SEQ ID NO: 393 - protein, 27 aa, Artificial Sequence
  immunoglobulin binding polypeptide
EHMCVYTTWGELIWCDNHEPGPEGGGK

SEQ ID NO: 394 - protein, 25 aa, Artificial Sequence
  immunoglobulin binding polypeptide
ACVYTTWGELIWCDNHEPGPEGGGK

SEQ ID NO: 395 - protein, 25 aa, Artificial Sequence
  immunoglobulin binding polypeptide
TCVYTTWGELIWCDNHEPGPEGGGK

SEQ ID NO: 396 - protein, 25 aa, Artificial Sequence
  immunoglobulin binding polypeptide
ECVYTTWGELIWCDNHEPGPEGGGK

SEQ ID NO: 397 - protein, 25 aa, Artificial Sequence
  immunoglobulin binding polypeptide
VCVYTTWGELIWCDNHEPGPEGGGK

SEQ ID NO: 398 - protein, 25 aa, Artificial Sequence
  immunoglobulin binding polypeptide
XCVYTTWGELIWCDNHEPGPEGGGK

SEQ ID NO: 399 - protein, 24 aa, Artificial Sequence
  immunoglobulin binding polypeptide
CVYTTWGELIWCDNHEPGPEGGGK

SEQ ID NO: 400 - protein, 27 aa, Artificial Sequence
  immunoglobulin binding polypeptide
SRACSRDWSGALVWCAGHEPGPEGGGK

SEQ ID NO: 401 - protein, 27 aa, Artificial Sequence
  immunoglobulin binding polypeptide
RRACSRDWSGALVWCAGHEPGPEGGGK

SEQ ID NO: 402 - protein, 27 aa, Artificial Sequence
  immunoglobulin binding polypeptide
ERACSRDWSGALVWCAGHEPGPEGGGK

SEQ ID NO: 403 - protein, 25 aa, Artificial Sequence
  immunoglobulin binding polypeptide
ACSRDWSGALVWCAGHEPGPEGGGK

SEQ ID NO: 404 - protein, 25 aa, Artificial Sequence
  immunoglobulin binding polypeptide
TCSRDWSGALVWCAGHEPGPEGGGK

SEQ ID NO: 405 - protein, 25 aa, Artificial Sequence
  immunoglobulin binding polypeptide
ECSRDWSGALVWCAGHEPGPEGGGK

SEQ ID NO: 406 - protein, 25 aa, Artificial Sequence
  immunoglobulin binding polypeptide
VCSRDWSGALVWCAGHEPGPEGGGK

SEQ ID NO: 407 - protein, 25 aa, Artificial Sequence
  immunoglobulin binding polypeptide
GCSRDWSGALVWCAGHEPGPEGGGK

SEQ ID NO: 408 - protein, 24 aa, Artificial Sequence
  immunoglobulin binding polypeptide
CSRDWSGALVWCAGHEPGPEGGGK

SEQ ID NO: 409 - protein, 18 aa, Artificial Sequence
  immunoglobulin binding polypeptide
NPMCWRASWWEDAYCINH

SEQ ID NO: 410 - protein, 18 aa, Artificial Sequence
  immunoglobulin binding polypeptide
NPMCWRAHWWEDAYCINH

SEQ ID NO: 411 - protein, 18 aa, Artificial Sequence
  immunoglobulin binding polypeptide
EHMCVYTTWGELIWCDNH

SEQ ID NO: 412 - protein, 16 aa, Artificial Sequence
  immunoglobulin binding polypeptide
ACVYTTWGELIWCDNH

SEQ ID NO: 413 - protein, 16 aa, Artificial Sequence
  immunoglobulin binding polypeptide
TCVYTTWGELIWCDNH

SEQ ID NO: 414 - protein, 16 aa, Artificial Sequence
  immunoglobulin binding polypeptide
ECVYTTWGELIWCDNH

SEQ ID NO: 415 - protein, 16 aa, Artificial Sequence
  immunoglobulin binding polypeptide
VCVYTTWGELIWCDNH

SEQ ID NO: 416 - protein, 16 aa, Artificial Sequence
  immunoglobulin binding polypeptide
XCVYTTWGELIWCDNH

SEQ ID NO: 417 - protein, 18 aa, Artificial Sequence
  immunoglobulin binding polypeptide
SRACSRDWSGALVWCAGH

SEQ ID NO: 418 - protein, 18 aa, Artificial Sequence
  immunoglobulin binding polypeptide
ERACSRDWSGALVWCAGH

SEQ ID NO: 419 - protein, 16 aa, Artificial Sequence
  immunoglobulin binding polypeptide
ACSRDWSGALVWCAGH

SEQ ID NO: 420 - protein, 16 aa, Artificial Sequence
  immunoglobulin binding polypeptide
TCSRDWSGALVWCAGH

SEQ ID NO: 421 - protein, 16 aa, Artificial Sequence
  immunoglobulin binding polypeptide
ECSRDWSGALVWCAGH

SEQ ID NO: 422 - protein, 16 aa, Artificial Sequence
  immunoglobulin binding polypeptide
VCSRDWSGALVWCAGH

SEQ ID NO: 423 - protein, 16 aa, Artificial Sequence
  immunoglobulin binding polypeptide
GCSRDWSGALVWCAGH

SEQ ID NO: 424 - protein, 15 aa, Artificial Sequence
  template sequence
XXXCXXXXXXXCXXX

SEQ ID NO: 425 - protein, 18 aa, Artificial Sequence
  Exemplary motif
XRXCXTXXPXXXXXCXXX

SEQ ID NO: 426 - protein, 18 aa, Artificial Sequence
  examplary motif
XXXCITXPFXXXXXCXNX

SEQ ID NO: 427 - protein, 12 aa, Artificial Sequence
  immunoglobulin segment
CVYTTWGELIWC

SEQ ID NO: 428 - protein, 12 aa, Artificial Sequence
  immunoglobulin segment
CSRDWSGALVWC

SEQ ID NO: 429 - protein, 12 aa, Artificial Sequence
  immunoglobulin segment
CSTSYWYYQWFC

SEQ ID NO: 430 - protein, 18 aa, Artificial Sequence
  serum albumin - binding agent
RNMCKFSWIRSPAFCARA